(19) United States
(12) Patent Application Publication
(10) Pub. No.: US 2019/0298740 A1
(43) Pub. Date: Oct. 3, 2019

(54) METHODS AND COMPOSITIONS FOR TREATING HALLUCINATIONS AND CONDITIONS RELATED TO THE SAME

(71) Applicant: Enterin, Inc., Philadelphia, PA (US)

(72) Inventors: Denise Barbut, Philadelphia, PA (US); Michael Zasloff, Philadelphia, PA (US)

(73) Assignee: Enterin, Inc., Philadelphia, PA (US)

(21) Appl. No.: 16/363,173

(22) Filed: Mar. 25, 2019

Related U.S. Application Data
(60) Provisional application No. 62/648,661, filed on Mar. 27, 2018.
(60) Provisional application No. 62/789,437, filed on Jan. 7, 2019.

(51) Int. Cl. A61K 31/575; A61K 9/00; A61P 25/28

(52) U.S. Cl. CPC A61K 31/575; A61P 25/28; A61K 9/0043; A61K 9/0053

(57) ABSTRACT

This application relates to methods of treating, preventing and/or slowing the onset or progression of hallucinations and/or related symptoms caused by a variety disorders, with aminosterols or pharmaceutically acceptable salts or derivatives thereof.

### CROSS-REFERENCE TO RELATED APPLICATIONS

This application claims the priority benefits under 35 USC § 119 to U.S. provisional Application 62/648,661, filed Mar. 27, 2018, and U.S. provisional Application 62/789,437, filed Jan. 7, 2019, the entire contents of which are incorporated herein by reference in their entirety.

### FIELD OF THE INVENTION

This application relates to methods of treating, preventing, or improving disorders associated with hallucinations and/or hallucinations in human subjects. The methods comprise administering to a subject in need thereof an amino sterol, or a salt or derivative thereof.

### BACKGROUND OF THE INVENTION

Squalamine is a unique compound with a structure that was not previously seen in nature, being a bile acid coupled to a polyamine (spermidine):

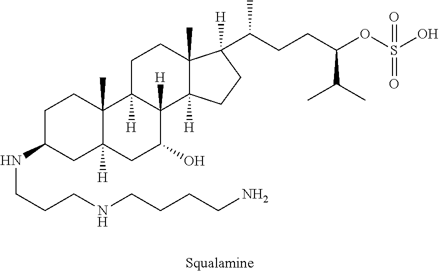

The discovery of squalamine, the structure of which is shown above, was reported by Michael Zasloff in 1993 (U.S. Pat. No. 5,192,756). Squalamine was discovered in various tissues of the dogfish shark (Squalus acanthias) in a search for antibacterial agents. The most abundant source of squalamine is in the livers of Squalus acanthias, although it is found in other sources, such as lampreys (Yun et al., 2007).

Aminosterol 1436 is an aminosterol isolated from the dogfish shark, which is structurally related to squalamine (U.S. Pat. No. 5,840,936; Rao et al., 2000).

There is a need in the art for new methods of treating hallucinations. The present invention satisfies this need.

### SUMMARY OF THE INVENTION

The present invention is directed to methods of treating, preventing, and/or slowing the onset or progression of hallucinations and/or a hallucination-related symptom in a subject in need comprising administering to the subject a composition comprising at least one aminosterol, or a salt or derivative thereof. Certain embodiments describe the determination and administration of a “fixed dose” of an aminosterol or a pharmaceutically acceptable salt or derivative thereof that is not age, size, or weight dependent but rather is individually calibrated.

The amino sterol or a salt or derivative thereof can be formulated with one or more pharmaceutically acceptable carriers or excipients. Preferably the aminosterol is a pharmaceutically acceptable grade of the amino sterol.

In one embodiment, the invention encompasses a method of treating, preventing and/or slowing the onset or progression of hallucinations and/or a related symptom in a subject in need comprising administering to the subject a therapeutically effective amount of at least one aminosterol or a salt or derivative thereof. In one aspect, the at least one aminosterol or a salt or derivative thereof is administered via any pharmaceutically acceptable means. Exemplary methods of administration include oral, nasal, sublingual, buccal, rectal, vaginal, intravenous, intra-arterial, intradermal, intraperitoneal, intrathecal, intramuscular, epidural, intracerebral, intracerebroventricular, transdermal, or any combination thereof. In another aspect, the at least one aminosterol or a salt or derivative thereof is administered nasally. In another aspect, administration of the at least one amino sterol or a salt or derivative thereof comprises non-oral administration.

The therapeutically effect amount of the at least one aminosterol or a salt or derivative thereof in the methods of the invention can be, for example, about 0.1 to about 20 mg/kg, about 0.1 to about 15 mg/kg, about 0.1 to about 10 mg/kg, about 0.1 to about 5 mg/kg, or about 0.1 to about 2.5 mg/kg body weight of the subject. In another aspect, the therapeutically effect amount of the at least one aminosterol or a salt or derivative thereof in the methods of the invention can be, for example, about 0.001 to about 500 mg/day, about 0.001 to about 375 mg per day, about 0.001 to about 250 mg/day, about 0.001 to about 125 mg/day, about 0.001 to about 50 mg/day, about 0.001 to about 25 mg/day, or about 0.001 to about 10 mg/day.

In another embodiment, where the method of administration comprises nasal administration, the therapeutically effective amount of the at least one amino sterol, or a salt or derivative thereof comprises about 0.001 to about 6 mg per day or about 0.001 to about 4 mg per day. Where the administration comprises oral administration, in another embodiment the therapeutically effective amount of the at least one amino sterol, or a salt or derivative thereof can comprise about 1 to about 300 mg per day or about 25 to about 300 mg per day.

In another embodiment, encompassed is a method of treating, preventing and/or slowing the onset or progression of hallucinations and/or a related symptom in a subject in need comprising (a) determining a dose of an amino sterol or a salt or derivative thereof for the subject, wherein the aminosterol dose is determined based on the effectiveness of the aminosterol dose in improving or resolving a hallucination symptom being evaluated, (b) followed by administering the aminosterol dose to the subject for a period of time, wherein the method comprises (i) identifying a hallucination symptom to be evaluated; (ii) identifying a starting aminosterol dose for the subject; and (iii) administering an escalating dose of the amino sterol to the subject over a period of time until an effective dose for the hallucination symptom being evaluated is identified, wherein the effective dose is the aminosterol dose where improvement or resolution of the hallucination symptom is observed, and fixing the amino sterol dose at that level for that particular hallucination symptom in that particular subject.

In aspect of the methods of the invention, the hallucinations are correlated with abnormal αS pathology and/or dopaminergic dysfunction. In addition, the hallucinations can comprise for example a visual, auditory, tactile, gustatory or olfactory hallucination. In another aspect, the hallucinations can be the result of a neurodegenerative disorder, a psychiatric disorder, a neurological disorder, a brain tumor, a sleep disorder, a focal brain lesion, a diffuse involvement of the cerebral cortex, a sensory loss; and/or dysfunction of the enteric nervous system.

Where the hallucinations are correlated with a neurodegenerative disorder, the neurodegenerative disorder can be for example synucleopathies, Parkinson's disease, Alzheimer's disease, dementia with Lewy bodies (DLB), multiple system atrophy (MSA), Huntington's Disease, Multiple Sclerosis (MS), Amyotorphic Lateral Sclerosis (ALS), schizophrenia, Friedreich's ataxia, vascular dementia, spinal muscular atrophy, supranuclear palsy, fronto temperal dementia (FTD), progressive supranuclear palsy, Guadeloupian Parkinsonism, parkinsonism, spinocerebellar ataxia, autism, stroke, traumatic brain injury, sleep disorders such as REM sleep behavior disorder (RBD), depression, down syndrome, Gaucher's disease (GD), Krabbe's disease (KD), lysosomal conditions affecting glycosphingolipid metabolism, ADHD, agitation, anxiety, delirium, irritability, illusion and delusions, amnesia, apathy, bipolar disorder, disinhibition, aberrant motor and obsessive-compulsive behaviors, addiction, cerebral palsy, epilepsy, major depressive disorder, degenerative processes associated with aging, and dementia of aging.

Where the hallucinations are correlated with a psychiatric disorder, the psychiatric disorder can be for example bipolar disorder, borderline personality disorder, depression (mixed), dissociative identity disorder, generalized anxiety disorder, major depression, obsessive compulsive disorder, post-traumatic stress disorder, psychosis (NOS), schizoaffective disorder, and schizophrenia. In addition, (a) the focal brain lesion can comprise occipital lobe lesions or temporal lobe lesions; (b) the temporal lobe lesion can be lesions of the uncinate gyrus, cerebral peduncles, and substantia nigra; (c) the diffuse involvement of the cerebral cortex is caused by a viral infectious disease; and/or (d) the diffuse involvement of the cerebral cortex is a result of a cerebral vasculitis condition.

Further, where the hallucinations are correlated with a viral disease, the viral infectious disease can be for example acute metabolic encephalopathies, encephalitis, and meningitis. Where the hallucinations are correlated with a cerebral vasculitis condition, then the cerebral vasculitis condition can be caused by an autoimmune disorder, a bacterial or viral infection, or a systemic vasculitis.

Where the hallucinations are caused by an autoimmune disorder, then the autoimmune disorder can be Systemic Lupus Erythematosus (SLE).

Examples of sensory loses that can result in hallucinations include, for example, visual, auditory, gustatory, tactile, and/or olfactory.

In one aspect of the methods of the invention, administration of the aminosterol reverses dysfunction: (a) of the neurodegenerative disorder and treats and/or prevents the hallucinations and/or related symptom; (b) of the psychiatric disorder and treats and/or prevents the hallucinations and/or related symptom; (c) of the neurological disorder and treats and/or prevents the hallucination; (d) of the sensory loss and treats the hallucination; and/or (e) of the enteric nervous system and treats the hallucination.

In another aspect, the methods result in a decreased number or severity of hallucinations of the subject, and/or the methods result in the subject being hallucination-free. For example, the methods can result in a decrease in the number of hallucinations, and the decrease in number of hallucinations can comprise a reduction in number of hallucinations over a defined period of time. In addition, the methods can result in a decreased severity of hallucinations over a defined period of time. The decreased severity of hallucinations can optionally be measured by a medically recognized technique selected from the group consisting of Chicago Hallucination Assessment Tool (CHAT), The Psychotic Symptom Rating Scales (PSYRATS), Auditory Hallucinations Rating Scale (AHRS), Hamilton Program for Schizophrenia Voices Questionnaire (HPSVQ), Characteristics of Auditory Hallucinations Questionnaire (CAHQ), Mental Health Research Institute Unusual Perception Schedule (MUPS), positive and negative syndrome scale (PANSS), scale for the assessment of positive symptoms (SAPS), Launay-Slade hallucinations scale (LSHS), the Cardiff anomalous perceptions scale (CAPS), and structured interview for assessing perceptual anomalies (SIAPA).

In all aspects of the methods described herein, each defined period of time can independently be about 1 day to about 10 days, about 10 days to about 30 days, about 30 days to about 3 months, about 3 months to about 6 months, about 6 months to about 12 months, or about greater than 12 months; or each defined period of time can be independently selected from about 1 day, about 1 week, about 2 weeks, about 3 weeks, about 1 month, about 1.5 months, about 2 months, about 2.5 months, about 3 months, about 3.5 months, about 4 months, about 4.5 months, about 5 months, about 5.5 months, or about 6 months.

In another aspect, in the methods of the invention the aminosterol or a salt or derivative thereof can be administered orally, intranasally, or a combination thereof.

In one embodiment, starting dosages of the aminosterol or a salt or derivative thereof for oral administration can range, for example, from about 1 mg up to about 175 mg/day, or any amount in-between these two values. In another embodiment, the composition is administered orally and the dosage of the amino sterol or a salt or derivative thereof is escalated in about 25 mg increments. In yet another embodiment, the composition is administered orally and the dose of the aminosterol or a salt or derivative thereof for the subject following dose escalation is fixed at a range of from about 1 mg up to about 500 mg/day, or any amount in-between these two values.

In another embodiment, the composition is administered intranasally (IN) and the starting amino sterol or a salt or derivative thereof dosage ranges from about 0.001 mg to about 3 mg/day, or any amount in-between these two values. For example, the starting aminosterol dosage for IN administration, prior to dose escalation, can be, for example, about 0.001, about 0.005, about 0.01, about 0.02, about 0.03, about 0.05, about 0.06, about 0.07, about 0.08, about 0.09, about 0.1, about 0.15, about 0.2, about 0.25, about 0.3, about 0.35, about 0.4, about 0.45, about 0.5, about 0.55, about 0.6, about 0.65, about 0.7, about 0.75, about 0.8, about 0.85, about 0.9, about 1.0, about 1.1, about 1.25, about 1.3, about 1.4, about 1.5, about 1.6, about 1.7, about 1.75, about 1.8, about 1.9, about 2.0, about 2.1, about 2.25, about 2.3, about 2.4, about 2.5, about 2.6, about 2.7, about 2.75, about 2.8, about 2.9, or about 3 mg/day.

In another embodiment, the composition is administered intranasally and the dosage of the aminosterol or a salt or derivative thereof is escalated in increments of about 0.01, about 0.05, about 0.1, about 0.2, about 0.25, about 0.3, about 0.35, about 0.4, about 0.45, about 0.5, about 0.55, about 0.6, about 0.65, about 0.7, about 0.75, about 0.8, about 0.85, about 0.9, about 0.95, about 1, about 1.1, about 1.2, about 1.3, about 1.4, about 1.5, about 1.6, about 1.7, about 1.8, about 1.9, or about 2 mg.

Finally, in yet another embodiment, the composition is administered intranasally and the dose of the amino sterol or a salt or derivative thereof for the subject following escalation is fixed at a range of from about 0.001 mg up to about 6 mg/day, or any amount in-between these two values. In yet a further embodiment, the aminosterol composition is administered intranasally and the dose of the aminosterol or a salt or derivative thereof for the subject following dose escalation is a dose which is sub therapeutic when given orally or by injection.

In one embodiment, the dosage of the amino sterol or a salt or derivative thereof is escalated every about 3 to about 5 days. In another embodiment, the dose of the aminosterol or a salt or derivative thereof is escalated about 1×/week, about 2×/week, about every other week, or about 1×/month. In yet another embodiment, the dose of the aminosterol or a salt or derivative thereof is escalated every about 1, about 2, about 3, about 4, about 5, about 6, about 7, about 8, about 9, about 10, about 11, about 12, about 13, or about 14 days.

In another embodiment, the fixed dose of the aminosterol or a salt or derivative thereof is given once per day, every other day, once per week, twice per week, three times per week, four times per week, five times per week, six times per week, every other week, or every few days. In addition, the fixed dose of the amino sterol or a salt or derivative thereof can be administered for a first defined period of time of administration, followed by a cessation of administration for a second defined period of time, followed by resuming administration upon recurrence of hallucinations or a symptom of hallucinations. For example, the fixed aminosterol dose can be incrementally reduced after the fixed dose of amino sterol or a salt or derivative thereof has been administered to the subject for a period of time. Alternatively, the fixed aminosterol dose is varied plus or minus a defined amount to enable a modest reduction or increase in the fixed dose. For example, the fixed aminosterol dose can be increased or decreased by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20%.

In another embodiment, the starting amino sterol or a salt or derivative thereof dose is higher if the hallucination symptom being evaluated is severe.

In one embodiment, the method results in slowing, halting, or reversing progression or onset of hallucinations over a defined period of time following administration of the fixed escalated dose of the aminosterol or a salt or derivative thereof, as measured by a medically-recognized technique. In addition, the method of the invention can result in positively impacting the hallucinations, as measured by a medically-recognized technique.

The positive impact and/or progression of hallucinations and/or related symptom can be measured quantitatively or qualitatively by one or more medically recognized techniques selected from the group consisting of Chicago Hallucination Assessment Tool (CHAT), The Psychotic Symptom Rating Scales (PSYRATS), Auditory Hallucinations Rating Scale (AHRS), Hamilton Program for Schizophrenia Voices Questionnaire (HPSVQ), Characteristics of Auditory Hallucinations Questionnaire (CAHQ), Mental Health Research Institute Unusual Perception Schedule (MUPS), positive and negative syndrome scale (PANSS), scale for the assessment of positive symptoms (SAPS), Launay-Slade hallucinations scale (LSHS), the Cardiff anomalous perceptions scale (CAPS), and structured interview for assessing perceptual anomalies (SIAPA). In addition, the progression or onset of hallucinations and/or related symptoms is slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by the one or more medically recognized techniques.

In the methods of the invention, administration of the aminosterol or a salt or derivative thereof can (a) reverse dysfunction caused by the hallucinations and treat, prevent, improve, and/or resolve the symptom being evaluated; (b) reverse dysfunction caused by the hallucinations and treat, prevent, improve, and/or resolve the symptom being evaluated and the improvement or resolution of the hallucination symptom is measured using a clinically recognized scale or tool; and/or (c) reverse dysfunction caused by the hallucinations and treat, prevent, improve, and/or resolve the symptom being evaluated and the hallucinations, by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, as measured using a clinically recognized scale.

In one aspect of the invention, the hallucination symptom to be evaluated is selected from the group consisting of: (a) a symptom from the Chicago Hallucination Assessment Tool (CHAT) selected from the group consisting of hallucination frequency, duration, sensory intensity, complexity, controllability, amount of negative content, degree of negative content, frequency of negative emotion associated with hallucination, intensity of emotional impact, and chronicity; (b) a symptom from the Mental Health Research Institute Unusual Perceptions Schedule (MUPS) selected from the group consisting of onset and course, number, volume, tone, and location; (c) auditory hallucination; (d) tactile hallucination; (e) visual hallucination; (f) olfactory hallucination; (g) gustatory hallucination; (h) delusions; (i) proprioceptive hallucination; (j) equilibrioceptive hallucination; (k) nociceptive hallucination; (l) thermoceptive hallucination; (m) chronoceptive hallucination; (n) non-auditory command hallucination; (o) psychosis; (p) peduncular hallucinosis; (p) delirium; (r) dementia; (s) neurodegenerative disease; (t) neurodegeneration; (u) epilepsy; (v) seizures; (w) migraines; (x) cognitive impairment; (y) constipation; (z) depression; (aa) sleep problem, sleep disorder, or sleep disturbance; and/or (bb) gastrointestinal disorders.

In one aspect of the methods described herein where the hallucination symptom to be evaluated is visual hallucination: (a) the method results in a decrease in number of visual hallucinations over a defined period of time; (b) the method results in a decrease in severity of visual hallucinations over a defined period of time, wherein the decrease in severity of visual hallucinations is measured quantitatively or qualitatively by one or more medically recognized techniques selected from the group consisting of Chicago Hallucination Assessment Tool (CHAT), The Psychotic Symptom Rating Scales (PSYRATS), Hamilton Program for Schizophrenia Voices Questionnaire (HPSVQ), Mental Health Research Institute Unusual Perception Schedule (MUPS), positive and negative syndrome scale (PANSS), scale for the assessment of positive symptoms (SAPS), Launay-Slade hallucinations scale (LSHS), the Cardiff anomalous perceptions scale (CAPS), and structured interview for assessing perceptual anomalies (SIAPA); and/or (c) the method results in the subject being visual hallucination-free.

In another aspect of the methods of invention where the hallucination symptom to be evaluated is auditory hallucination (a) the method results in a decrease in number of auditory hallucinations over a defined period of time; (b) the method results in a decrease in severity of auditory hallucinations over a defined period of time, wherein the decrease in severity of auditory hallucinations is measured quantitatively or qualitatively by one or more medically recognized techniques selected from the group consisting of Chicago Hallucination Assessment Tool (CHAT), The Psychotic Symptom Rating Scales (PSYRATS), Auditory Hallucinations Rating Scale (AHRS), Hamilton Program for Schizophrenia Voices Questionnaire (HPSVQ), Characteristics of Auditory Hallucinations Questionnaire (CAHQ), Mental Health Research Institute Unusual Perception Schedule (MUPS), positive and negative syndrome scale (PANSS), scale for the assessment of positive symptoms (SAPS), Launay-Slade hallucinations scale (LSHS), the Cardiff anomalous perceptions scale (CAPS), and structured interview for assessing perceptual anomalies (SIAPA); and/or (c) 6he method results in the subject being auditory hallucination-free.

In yet another aspect of the methods of the invention where the hallucination symptom to be evaluated is tactile hallucination, (a) the method results in a decrease in number of tactile hallucinations over a defined period of time; (b) the method results in a decrease in severity of tactile hallucinations over a defined period of time, wherein the decrease in severity of tactile hallucinations is measured quantitatively or qualitatively by one or more medically recognized techniques selected from the group consisting of Chicago Hallucination Assessment Tool (CHAT), The Psychotic Symptom Rating Scales (PSYRATS), Hamilton Program for Schizophrenia Voices Questionnaire (HPSVQ), Mental Health Research Institute Unusual Perception Schedule (MUPS), positive and negative syndrome scale (PANSS), scale for the assessment of positive symptoms (SAPS), Launay-Slade hallucinations scale (LSHS), the Cardiff anomalous perceptions scale (CAPS), and structured interview for assessing perceptual anomalies (SIAPA); and/or (c) the method results in the subject being tactile hallucination-free.

In one aspect of the methods where the hallucination symptom to be evaluated is olfactory hallucination (a) the method results in a decrease in number of olfactory hallucinations over a defined period of time; (b) the method results in a decrease in severity of olfactory hallucinations over a defined period of time, wherein the decrease in severity of olfactory hallucinations is measured quantitatively or qualitatively by one or more medically recognized techniques selected from the group consisting of Chicago Hallucination Assessment Tool (CHAT), The Psychotic Symptom Rating Scales (PSYRATS), Hamilton Program for Schizophrenia Voices Questionnaire (HPSVQ), Mental Health Research Institute Unusual Perception Schedule (MUPS), positive and negative syndrome scale (PANSS), scale for the assessment of positive symptoms (SAPS), Launay-Slade hallucinations scale (LSHS), the Cardiff anomalous perceptions scale (CAPS), and structured interview for assessing perceptual anomalies (SIAPA); and/or (c) the method results in the subject being olfactory hallucination-free.

In one embodiment, the “defined period of time” is about 1 day to about 10 days, about 10 days to about 30 days, about 30 days to about 3 months, about 3 months to about 6 months, about 6 months to about 12 months, or about greater than 12 months. In addition, the decrease in number of hallucination can be, for example, is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. In another aspect, the decrease in severity of hallucinations is measured quantitatively and is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In some embodiments, the hallucination symptom to be evaluated is cognitive impairment, and (a) progression or onset of the cognitive impairment is slowed, halted, or reversed over a defined period of time following administration of the fixed escalated dose of the aminosterol or a salt or derivative thereof, as measured by a medically-recognized technique; (b) the cognitive impairment is positively impacted by the fixed escalated dose of the aminosterol or a salt or derivative thereof, as measured by a medically-recognized technique; (c) the cognitive impairment is positively impacted by the fixed escalated dose of the aminosterol or a salt or derivative thereof, as measured by a medically-recognized technique and the positive impact on and/or progression of cognitive impairment is measured quantitatively or qualitatively by one or more techniques selected from the group consisting of ADASCog, Mini-Mental State Exam (MMSE), Mini-cog test, Woodcock-Johnson Tests of Cognitive Abilities, Leiter International Performance Scale, Miller Analogies Test, Raven's Progressive Matrices, Wonderlic Personnel Test, IQ tests, or a computerized tested selected from Cantab Mobile, Cognigram, Cognivue, Cognision, and Automated Neuropsychological Assessment Metrics Cognitive Performance Test (CPT); and/or (d) the progression or onset of cognitive impairment is slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique.

In some embodiments, the hallucination symptom to be evaluated is constipation, and (a) treating the constipation prevents and/or delays the onset and/or progression of the hallucinations; (b) the fixed escalated aminosterol dose causes the subject to have a bowel movement; (c) the method results in an increase in the frequency of bowel movement in the subject; (d) the method results in an increase in the frequency of bowel movement in the subject and the increase in the frequency of bowel movement is defined as: (i) an increase in the number of bowel movements per week of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (ii) a percent decrease in the amount of time between each successive bowel movement selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; (e) as a result of the method the subject has the frequency of bowel movement recommended by a medical authority for the age group of the subject; and/or (f) the starting amino sterol dose is determined by the severity of the constipation, wherein: (i) if the average complete spontaneous bowel movement (CSBM) or spontaneous bowel movement (SBM) is one or less per week, then the starting aminosterol dose is at least about 150 mg; and (ii) if the average CSBM or SBM is greater than one per week, then the starting aminosterol dose is about 75 mg or less.

In another embodiment, the hallucination symptom to be evaluated is a sleep problem, sleep disorder, and/or sleep disturbance, and wherein: (a) treating the sleep problem, sleep disorder, sleep disturbance prevents or delays the onset and/or progression of the hallucination and/or related symptom; (b) the sleep disorder or sleep disturbance comprises a delay in sleep onset, sleep fragmentation, REM-behavior disorder, sleep-disordered breathing including snoring and apnea, day-time sleepiness, micro-sleep episodes, narcolepsy, hallucinations, or any combination thereof, and optionally where the REM-behavior disorder comprises vivid dreams, nightmares, and acting out the dreams by speaking or screaming, or fidgeting or thrashing of arms or legs during sleep; (d) the method results in a positive change in the sleeping pattern of the subject; (e) the method results in a positive change in the sleeping pattern of the subject, wherein the positive change is defined as: (i) an increase in the total amount of sleep obtained of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (ii) a percent decrease in the number of awakenings during the night selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; and/or (f) as a result of the method the subject obtains the total number of hours of sleep recommended by a medical authority for the age group of the subject.

In another embodiment, the hallucination symptom to be evaluated is depression. In an exemplary embodiment, treating the depression prevents and/or delays the onset and/or progression of the hallucinations and/or related symptom. In another aspect, the method results in improvement in a subject's depression, as measured by one or more clinically-recognized depression rating scales. For example, the improvement can be in one or more depression characteristics selected from the group consisting of mood, behavior, bodily functions such as eating, sleeping, energy, and sexual activity, and/or episodes of sadness or apathy. In another embodiment, the improvement a subject experiences following treatment can be about 5, about 10, about 15, about 20, about 25, about 30, about 35, about 40, about 45, about 50, about 55, about 60, about 65, about 70, about 75, about 80, about 85, about 90, about 95 or about 100%.

In one embodiment, the schizophrenia symptom to be evaluated is neurodegeneration correlated with hallucinations, and (a) treating the neurodegeneration prevents and/or delays the onset and/or progression of the hallucinations; and/or (b) the method results in treating, preventing, and/or delaying the progression and/or onset of neurodegeneration in the subject. In an exemplary embodiment (a) progression or onset of the neurodegeneration is slowed, halted, or reversed over a defined period of time following administration of the fixed escalated dose of the aminosterol or a salt or derivative thereof, as measured by a medically-recognized technique; and/or (b) the neurodegeneration is positively impacted by the fixed escalated dose of the aminosterol or a salt or derivative thereof, as measured by a medically-recognized technique. The positive impact and/or progression of neurodegeneration can be measured quantitatively or qualitatively by one or more techniques selected from the group consisting of electroencephalogram (EEG), neuroimaging, functional MRI, structural MRI, diffusion tensor imaging (DTI), [18F]fluorodeoxyglucose (FDG) PET, agents that label amyloid, [18F]F-dopa PET, radiotracer imaging, volumetric analysis of regional tissue loss, specific imaging markers of abnormal protein deposition, multimodal imaging, and biomarker analysis. In addition, the progression or onset of neurodegeneration can be slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique.

In another embodiment, the aminosterol or a salt or derivative thereof is administered in combination with at least one additional active agent to achieve either an additive or synergistic effect. For example, the additional active agent can be administered via a method selected from the group consisting of (a) concomitantly; (b) as an admixture; (c) separately and simultaneously or concurrently; or (d) separately and sequentially. In another embodiment, the additional active agent is a different amino sterol from that administered in primary method. In yet a further embodiment, the method of the invention comprises administering a first aminosterol which is aminosterol 1436 or a salt or derivative thereof intranasally and administering a second amino sterol which is squalamine or a salt or derivative thereof orally.

In another embodiment, the at least one additional active agent is an active agent used to treat hallucinations or a symptom thereof, such as first-generation antipsychotics such as chlorpromazine (Thorazine®), fluphenazine (Prolixin®), haloperidol (Haldol®), perphenazine (Trilafon®), thioridazine (Mellaril®), thiothixene (Navane®), and trifluoperazine (Stelazine®); atypical antipsychotics such as aripiprazole (Abilify®), aripiprazole lauroxil (Aristada®), asenapine (Saphris®), clozapine (Clozaril®), iloperidone (Fanapt®), lurasidone (Latuda®), olanzapine (Zyprexa®), paliperidone (Invega Sustenna®), paliperidone palmitate (Invega Trina®), quetiapine (Seroquel®), risperidone (Risperdal®), pimavanserin and ziprasidone (Geodon®).

For all of the methods of the invention, in one embodiment each amino sterol dose is taken on an empty stomach, optionally within about two hours of the subject waking. In another embodiment for all of the methods of the invention, no food is taken or consumed after about 60 to about 90 minutes of taking the amino sterol dose. Further, in yet another embodiment applicable to all of the methods of the invention, the aminosterol or a salt or derivative thereof can be a pharmaceutically acceptable grade of at least one aminosterol or a pharmaceutically acceptable salt or derivative thereof. For all of the methods of the invention the subject can be a human.

In another embodiment, the subject to be treated according to the methods of the invention can be a member of a patient population at risk for being diagnosed with hallucinations.

The amino sterol or a salt or derivative thereof utilized in the methods of the invention can be, for example, (a) isolated from the liver of Squalus acanthias; (b) a synthetic amino sterol; (c) squalamine or a pharmaceutically acceptable salt thereof; (d) a squalamine isomer; (e) the phosphate salt of squalamine; (f) aminosterol 1436 or a pharmaceutically acceptable salt thereof; (g) an aminosterol 1436 isomer; (h) the phosphate salt of aminosterol 1436; (i) a compound comprising a sterol nucleus and a polyamine attached at any position on the sterol, such that the molecule exhibits a net charge of at least +1; (j) a compound comprising a bile acid nucleus and a polyamine, attached at any position on the bile acid, such that the molecule exhibits a net charge of at least +1; (k) a derivative modified to include one or more of the following: (i) substitutions of the sulfate by a sulfonate, phosphate, carboxylate, or other anionic moiety chosen to circumvent metabolic removal of the sulfate moiety and oxidation of the cholesterol side chain; (ii) replacement of a hydroxyl group by a non-metabolizable polar substituent, such as a fluorine atom, to prevent its metabolic oxidation or conjugation; and (iii) substitution of one or more ring hydrogen atoms to prevent oxidative or reductive metabolism of the steroid ring system; and/or (l) a derivative of squalamine or aminosterol 1436 modified through medicinal chemistry to improve bio-distribution, ease of administration, metabolic stability, or any combination thereof. In one embodiment, the aminosterol is selected from the group consisting aminosterol 1436 or a pharmaceutically acceptable salt thereof, squalamine or a pharmaceutically acceptable salt thereof, or a combination thereof. In another embodiment, the aminosterol is a phosphate salt.

In another embodiment, the aminosterol in the methods of the invention is selected from the group consisting of:

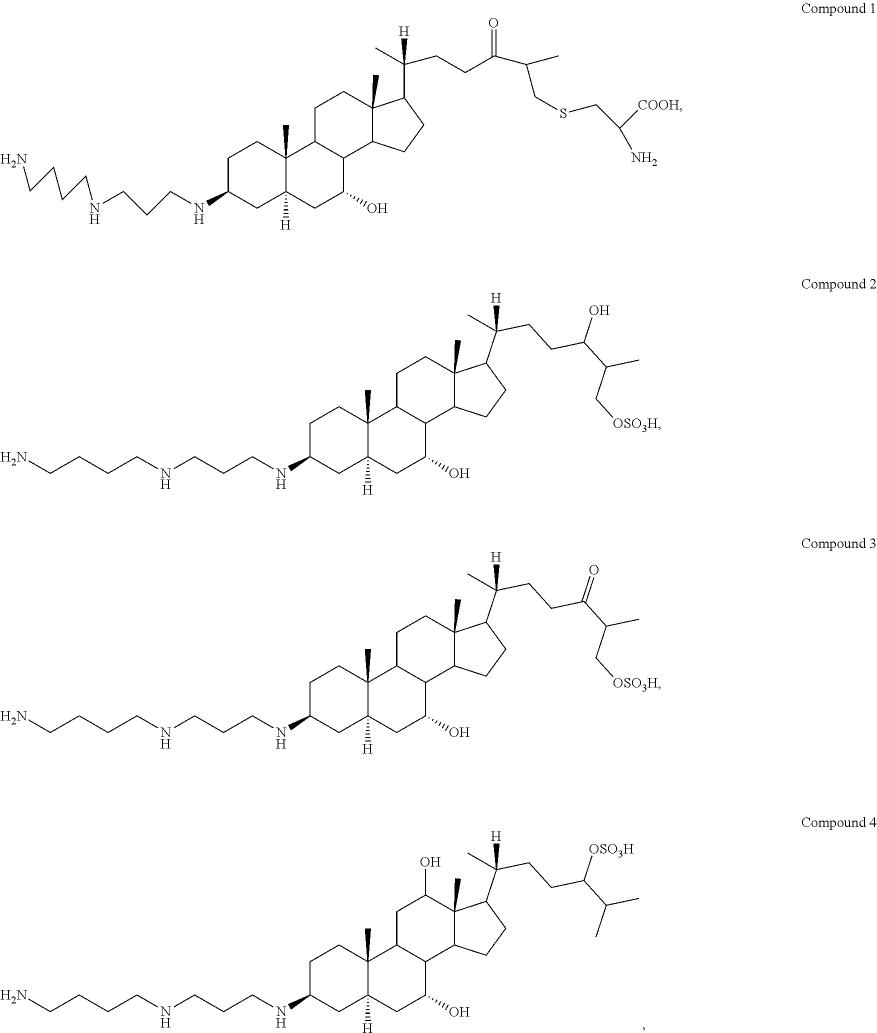

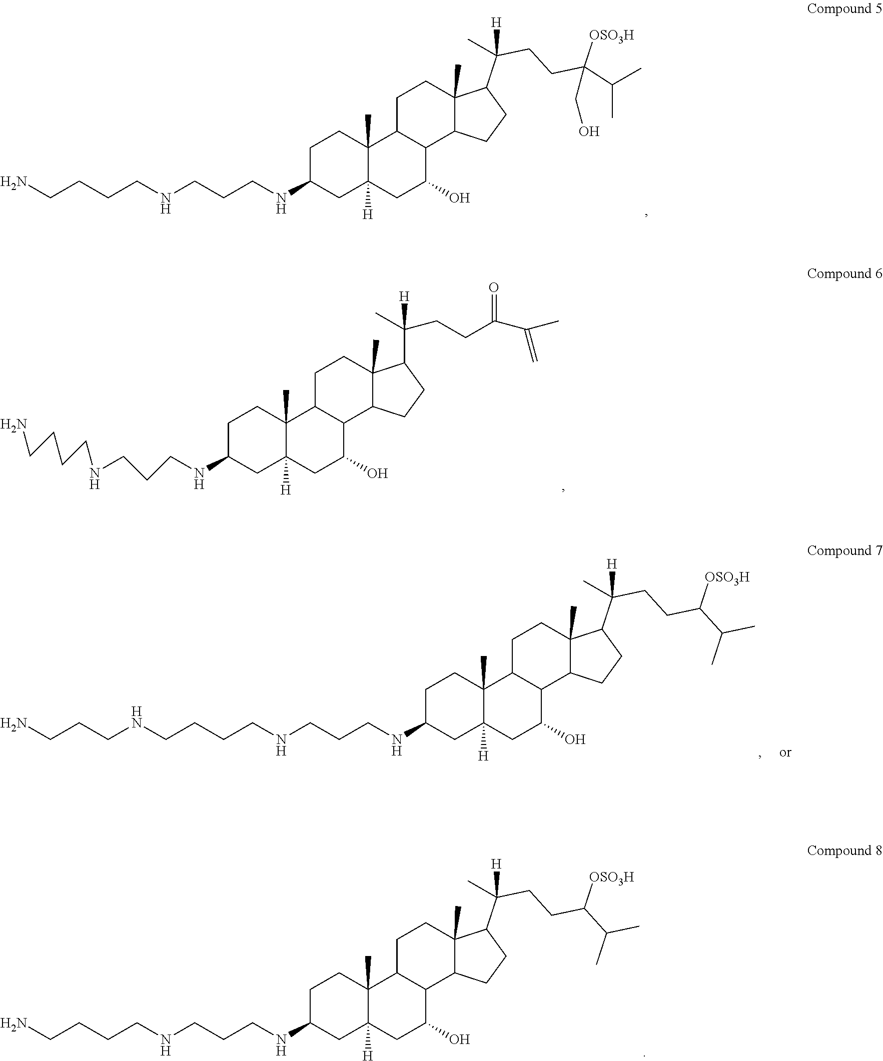

Further, the aminosterol composition can comprise, for example, one or more of the following: an aqueous carrier, a buffer, a sugar, and/or a polyol compound.

Both the foregoing summary of the invention and the following brief description of the drawings and the detailed description of the invention are exemplary and explanatory and are intended to provide further details of the invention as claimed. Other objects, advantages, and novel features will be readily apparent to those skilled in the art from the following detailed description of the invention.

### BRIEF DESCRIPTION OF THE DRAWINGS

FIGS. 1A and 1B show prokinetic activity of squalamine (ENT-01, a synthetic squalamine salt comprising squalamine as the active ion). As shown in FIG. 1A, in Stage 1 of the clinical trial (single dose), cumulative prokinetic response rate was defined as the proportion of patients who had a complete spontaneous bowel movements (CSBM) within 24 hours of dosing. In Stage 2 of the clinical trial (daily dosing), a prokinetic response was defined as the fraction of patients who had a CSBM within 24 hours of dosing on at least 2 out of 3 days at any given dose. As shown in FIG. 1B, the prokinetic dose of squalamine was significantly related to baseline constipation severity (p=0.00055). Patients with baseline CSBM <1 required a higher dose (mean, 192 mg) of squalamine than patients with CSBM ≥1 (mean, 120 mg).

FIG. 2 is a schematic (flowchart) showing patient disposition in Stage 2. (1) Patients first enrolled (n=40); (2) 6 patients failed to meet dosing criteria and were excluded; (3) 34 patients were dosed; (4) 5 patients were discontinued; 3 patients withdrew consent (with 1 patient lost to follow up and 2 patients withdrew because of diarrhea); and 2 patients discontinued because of an adverse event (recurrent dizziness after medication); (5) 31 patients had an assessable prokinetic response; and (6) 29 patients completed dosing.

FIG. 3 is a chart of total sleep time in relation to squalamine dose. Total sleep time was obtained from the sleep diary by subtracting awake time during the night from total time spent in bed. Total sleep time per night was logged for each patient at baseline, each dosing period and at washout, and the means were determined. The light grey bar represents the baseline value for each cohort at a given dose level and the dark grey bar represents the value for the same cohort at the stated dose of squalamine (ENT-01; Kenterin™). The number of patients represented at each value are: Baseline, 33; 75 mg, 21; 100 mg, 28; 125 mg, 18; 150 mg, 15; 175 mg, 12; 200 mg, 7; 225 mg, 3; 250 mg, 2; washout, 33. P values were as follows: 75 mg, p=0.4; 100 mg, p=0.1; 125 mg, p=0.3; 150 mg, p=0.07; 175 mg, p=0.03; 200 mg, p=0.3; 225 mg, p=0.5; 250 mg, p=0.3; wash-out, p=0.04 (paired t test).

FIG. 4 shows total sleep time vs the dose of squalamine (ENT-01), with total sleep time increasing progressively from baseline to 250 mg.

FIG. 5 shows total sleep time vs the dose of squalamine (ENT-01), with total sleep time increasing progressively from baseline to 250 mg.

FIG. 6 shows the effect of squalamine (ENT-01) on circadian rhythm. The figure depicts the mean waveform of temperature under three conditions per patient: baseline (Line #1), treatment with highest drug dose (Line #2), and washout (Line #3). Each mean waveform is double plotted for better visualization. Low temperatures indicate higher activation, while higher values are associated with drowsiness and sleepiness. The top black bar indicates a standard rest period from 23:00 to 07:00 h.

FIGS. 7A-F show the effect of squalamine (ENT-01) on circadian rhythm. The figures depict the results of circadian non-parametric analysis of wrist skin temperature rhythm throughout each condition (baseline, treatment with highest dose of squalamine (ENT-01) and washout). The following parameters were measured: Inter-daily variability (FIG. 7A), inter-daily stability (IS) (FIG. 7B), relative amplitude (RA) (FIG. 7C), circadian function index (FIG. 7D), M5V (FIG. 7E), which refers to the five consecutive hours with the highest temperature or high somnolence, and L10V (FIG. 7F), which indicates the mean of the ten consecutive hours with lowest temperature or high activation. The circadian function index (CFI) is an integrated score that ranges from 0 (absence of circadian rhythm) to 1 (robust circadian rhythm). Student's paired t-test, *p<0.05, **p<01, ***p<0.001.Values expressed as mean±SEM (n=12 in each condition).

FIG. 8 shows REM-behavior disorder in relation to squalamine (ENT-01) dose, with arm and leg thrashing episodes (mean values) calculated using sleep diaries. The frequency of arm or leg thrashing reported in the sleep diary diminished progressively from 2.2 episodes/week at baseline to 0 at maximal dose.

### DETAILED DESCRIPTION OF THE INVENTION

### I. Overview

The present invention is directed to methods of treating, preventing, and/or slowing the onset or progression of hallucinations and/or a hallucination-related symptom in a subject in need thereof. In one embodiment, the invention is directed to methods of treating, preventing, and/or slowing the onset or progression of hallucinations and/or a hallucination-related symptom correlated with abnormal α-synuclein (αS) pathology. The methods comprise administering one or more amino sterols or pharmaceutically acceptable salts or derivatives thereof to a subject in need. The discovery described herein that administration of an aminosterol or a salt or derivative thereof is necessary to achieve amelioration of hallucinations is particularly surprising since these compounds are believed to have poor bioavailability when delivered orally.

It is known that αS is an important presynaptic protein regulating critical aspects of dopamine (DA) neurotransmission. Thus, the present invention is also directed to methods of treating, preventing, and/or slowing the onset or progression of hallucinations and/or a hallucination-related symptom correlated with conditions related to dysfunctional DA neurotransmission, also known as dopaminergic dysfunction.

Examples of conditions or disorders correlated with hallucinations and/or related symptoms, and which are also correlated with abnormal αS pathology, and/or dopaminergic dysfunction, include but are not limited to neurodegenerative diseases associated with neural cell death, psychological or behavior disorders, and cerebral and general ischemic disorders, as described in detail below.

In one embodiment, the present invention is directed to methods of treating, preventing, and/or slowing the onset or progression of hallucinations and/or a hallucination-related symptom, comprising: (a) determining a dose of an aminosterol or a salt or derivative thereof for the subject, wherein the amino sterol dose is determined based on the effectiveness of the aminosterol dose in improving or resolving a hallucination symptom being evaluated; (b) followed by administering the dose of the aminosterol or a salt or derivative thereof to the subject for a period of time. The method of determining the aminosterol dose comprises (i) identifying a hallucination symptom to be evaluated; (ii) identifying a starting aminosterol dose for the subject; and (iii) administering an escalating dose of the amino sterol to the subject over a period of time until an effective dose for the hallucination symptom being evaluated is identified, wherein the effective dose is the aminosterol dose where improvement or resolution of the hallucination symptom is observed, and fixing the aminosterol dose at that level for that particular hallucination symptom in that particular subject.

Extensive studies in animals have shown that neither squalamine nor Aminosterol 1436, which are both aminosterols, can be absorbed to any extent from the gastrointestinal tract (GIT), requiring parenteral administration for the various previously conceived applications of these compounds. Moreover, consistent with its poor bioavailability when delivered orally, Aminosterol 1436, although capable of inducing weight loss when administered parenterally to dogs and rodents, exhibited no anorectic activity. Indeed, in a published review on the applications of squalamine as a therapeutic, Genaera scientists state “[a]lthough squalamine lactate is well absorbed in rodents by the subcutaneous and intraperitoneal routes, preliminary studies indicate that it is poorly bioavailable orally.” Connolly et al., 2006. Furthermore, squalamine and related aminosterols, such as 1436, do not exit the GIT into either the portal or systemic blood stream. This resulted in generally accepted conclusions that squalamine (and other amino sterols) could not provide a benefit for the treatment of systemic conditions. Thus, it was entirely unknown that aminosterols, such as Aminosterol 1436 and squalamine and salts and derivatives thereof, could be administered for the treatment of hallucinations or conditions associated with hallucinations.

A. Types of Hallucinations and Diseases Associated with the Same

A hallucination is a sensory impression or perception of an object or event, in any of the five senses (sight, touch, sound, smell, or taste) that has no basis in external stimulation. Hallucinations can have debilitating impact on the subject's health and life by causing harm to self or others, by making it difficult for the subject to function normally in everyday situations, and by causing sleep disruption. Examples of hallucinations include “seeing” someone not there (visual hallucination), “hearing” a voice not heard by others (auditory hallucination), “feeling” something crawling up your leg (tactile hallucination), “smelling” (olfactory), and “tasting” (gustatory). Other examples of hallucination types include hypnagogic hallucination (a vivid, dreamlike hallucination occurring at sleep onset), hypnopompic hallucination (a vivid, dreamlike hallucination occurring on awakening), kinesthetic hallucination (a hallucination involving the sense of bodily movement), and somatic hallucination, a hallucination involving the perception of a physical experience occurring within the body.

A paracusia, or auditory hallucination, is a form of hallucination that involves perceiving sounds without auditory stimulus. A common form of auditory hallucination involves hearing one or more talking voices. This may be associated with psychotic disorders; however, individuals without any psychiatric disease whatsoever may hear voices. There are three main categories into which the hearing of talking voices often fall: a person hearing a voice speak one's thoughts, a person hearing one or more voices arguing, or a person hearing a voice narrating his/her own actions. Other types of auditory hallucination include exploding head syndrome and musical ear syndrome. In the latter, people will hear music playing in their mind, usually songs they are familiar with. This can be caused by: lesions on the brain stem (often resulting from a stroke); also, sleep disorders such as narcolepsy, tumors, encephalitis, or abscesses. This should be distinguished from the commonly experienced phenomenon of getting a song stuck in one's head. Reports have also mentioned that it is also possible to get musical hallucinations from listening to music for long periods of time. Other reasons include hearing loss and epileptic activity. Prior studies have reported voice hearing in persons with a wide variety of DSM-5 diagnoses, including bipolar disorder, borderline personality disorder, depression (mixed), dissociative identity disorder, generalized anxiety disorder, major depression, obsessive compulsive disorder, post-traumatic stress disorder, psychosis (NOS), schizoaffective disorder, and schizophrenia. However, numerous persons surveyed reported no diagnosis.

Tactile hallucination is the false perception of tactile sensory input that creates a hallucinatory sensation of physical contact with an imaginary object. It is caused by the faulty integration of the tactile sensory neural signals generated in the spinal cord and the thalamus and sent to the primary somatosensory cortex (SI) and secondary somatosensory cortex (SII). Tactile hallucinations are recurrent symptoms of neurological diseases such as schizophrenia, Parkinson's disease, Ekbom's syndrome and delirium tremens. Patients who experience phantom limb pains also experience a type of tactile hallucination. Tactile hallucinations are also caused by drugs such as cocaine and alcohol.

An olfactory hallucination (phantosmia) makes an individual detect smells that aren't really present in their environment. The odors detected in phantosmia vary from person to person and may be foul or pleasant. They can occur in one or both nostrils. The phantom smell may seem to always be present or it may come and go. Phantosmia may occur after a head injury or upper respiratory infection. It can also be caused by temporal lobe seizures, inflamed sinuses, brain tumors and Parkinson's disease.

Hallucinations can be a result of psychiatric conditions. Hallucinations, especially auditory hallucinations, are characteristic of certain psychiatric conditions such as schizophrenia, occurring in up to 70-80% of subjects. They also occur in 30-50% of individuals with borderline personality disorder. Auditory hallucinations can take control of actions or behavior and elicit violent defensive behavior or alternatively lead to self-harming behavior (Yee et al., 2005). They can also occur in post-partum psychosis. The voices can order the mother to kill her baby or accuse her of being a bad mother. Auditory hallucinations can less commonly occur in severely depressed patients or even in mania. Substance abuse can also be associated with visual hallucinations. Typically the hallucinations are simple geometric shapes and vivid colors but formed tactile hallucinations such as insects crawling up a leg can occur in amphetamine and cocaine induced psychosis. Alcohol intoxication or withdrawal, post-traumatic stress disorder (PTSD) and bereavement can also be associated with visual hallucinations.

Hallucinations can be a result of neurological disorders. Neurological disorders cover a wide range of damage to brain tissue. The neurological disorder can be caused by brain tumors. The neurological disorder can be caused by sleep disorders such as narcolepsy. Furthermore, neurological disorders may be a variety of focal brain lesions, resulting in particular types of hallucinations depending on the location on the lesion. Formed and unformed visual hallucinations can occur in the presence of temporal and occipital lobe lesions in the brain. Occipital lobe lesions typically produce simple geometric patterns or “strings of circles like a bunch of grapes” or stars which can follow the gaze (palinopsia), whereas temporal lobe lesions are associated with complex, formed hallucinations. Temporal lobe lesions and especially lesions of the uncinate gyrus are typically associated with olfactory and gustatory hallucinations. Lesions of the cerebral peduncles and substantia nigra are associated with “peduncular hallucinosis” or colorful vivid images.

Hallucinations may be a result of diffuse involvement of the cerebral cortex. In some embodiments, the diffuse involvement of the cerebral cortex may be caused by a viral infectious disease. In some embodiments, the viral infectious disease is selected from the group consisting of acute metabolic encephalopathies, encephalitis, and meningitis. In other embodiments, the diffuse involvement of the cerebral cortex may be a result of a cerebral vasculitis condition. The cerebral vasculitis condition can be caused by autoimmune disorders, bacterial or viral infection, or systemic vasculitis. In one embodiment, the autoimmune disorder is Systemic Lupus Erythematosus (SLE).

Hallucinations can be caused by neurodegenerative disorders, including for example synucleopathies, Parkinson's disease, Alzheimer's disease, dementia with Lewy bodies (DLB), multiple system atrophy (MSA), Huntington's Disease, Multiple Sclerosis (MS), Amyotorphic Lateral Sclerosis (ALS), schizophrenia, Friedreich's ataxia, vascular dementia, spinal muscular atrophy, supranuclear palsy, fronto temporal dementia (FTD), progressive supranuclear palsy, Guadeloupian Parkinsonism, Parkinsonism, spinocerebellar ataxia, autism, stroke, traumatic brain injury, sleep disorders such as REM sleep behavior disorder (RBD), depression, down syndrome, Gaucher's disease (GD), Krabbe's disease (KD), lysosomal conditions affecting glycosphingolipid metabolism, ADHD, agitation, anxiety, delirium, irritability, illusion and delusions, amnesia, apathy, bipolar disorder, disinhibition, aberrant motor and obsessive-compulsive behaviors, addiction, cerebral palsy, epilepsy, major depressive disorder, degenerative processes associated with aging, and dementia of aging.

Hallucinations can be caused by neurological disorders such as, for example, (a) a brain tumor, (b) a sleep disorder such as narcolepsy or REM sleep behavior disorder (RBD), or (c) a focal brain lesion, such as occipital lobe lesions or temporal lobe lesions. In an exemplary embodiment, the temporal lobe lesion can be lesions of the uncinate gyrus, cerebral peduncles, or substantia nigra. The neurological disorder can be, for example, the result of (d) a diffuse involvement of the cerebral cortex, such as that caused by a viral infectious disease. For example, the viral infectious disease can be selected from the group consisting of acute metabolic encephalopathies, encephalitis, and meningitis. In another embodiment, the diffuse involvement of the cerebral cortex is a result of a cerebral vasculitis condition. For example, the cerebral vasculitis condition can be caused by an autoimmune disorder, a bacterial or viral infection, or a systemic vasculitis. For example, the autoimmune disorder can be Systemic Lupus Erythematosus (SLE).

Hallucinations can be caused by psychiatric disorders such as, for example, bipolar disorder, borderline personality disorder, depression, depression (mixed), dissociative identity disorder, generalized anxiety disorder, major depression, major depressive disorder, obsessive compulsive disorder, aberrant motor and obsessive-compulsive behaviors, addiction, post-traumatic stress disorder, psychosis (NOS), schizoaffective disorder, ADHD, agitation, anxiety, delirium, irritability, illusion and delusions, amnesia, apathy, and schizophrenia.

Hallucinations can frequently occur in hospitalized individuals with borderline dementia and are exacerbated in dim light, a condition referred to as “sun-downing.” All of these diseases are commonly associated with visual and sometimes tactile hallucinations, particularly as a late feature of the diseases. In PD, hallucinations typically involve faceless people, often dead relatives and are typically non-threatening in nature. The brain structures most severely affected in these conditions are the amygdala, hippocampus, mesial and lateral temporal lobes.

Hallucinations can be caused by sensory loss. Progressive visual loss and blindness can be associated with visual hallucinations (Charcot-Bonnet syndrome) and is exacerbated by dim light. Hallucinations caused by sensory loss can be simple or complex. Hallucinations have also been reported in individuals with congenital blindness. Auditory hallucinations can occur in individuals with hearing loss and deafness and can be unilateral or bilateral. Hallucinations can also occur in congenitally deaf individuals.

Hallucinations can be caused by dysfunction of the enteric nervous system. There is a growing realization that cross-talk between the enteric and central nervous system forms a gut-brain axis that plays a key role in the biological and physiological basis of neurodevelopmental, age-related, and neurodegenerative disorders. Indeed, it has been suggested that the pathology of Parkinson's disease (PD) starts in the gut and spread towards the central nervous system, and studies indicate that the enteric nervous system is frequently involved in the pathology of PD due to the effects of α-synuclein (Miraglia et al., 2015). Consistent with the fact that α-synuclein deposits can cause hallucinations, α-synuclein deposits in the stratum griseum intermedium, an important structure in directing attention toward visual targets, were observed in dementia with Lewy bodies patients that exhibits visual hallucinations, but not in Alzheimer's patients without visual hallucinations. (Erskine et al., 2017).

B. Hallucinations and Abnormal αS pathology

Many neurodiseases causing hallucinations such as PD are suspected to correlate with the formation of toxic αS aggregates within the enteric nervous system (ENS) (Braak et al., 2003). As a result of the normal trafficking of αS aggregates from the ENS to the central nervous system (CNS) via afferent nerves such as the vagus (Holmqvist et al., 2014; Svensson et al., 2015), neurotoxic aggregates accumulate progressively within the brainstem and more rostral structures. Inhibiting αS aggregation in the ENS may, thus, reduce the continuing neurodisease process in both the ENS and CNS (Phillips et al., 2008), and thereby positively impact hallucinations associated with abnormal αS pathology.

αS is a member of the synuclein family of soluble proteins (αS, β-synuclein and γ-synuclein) that are commonly present in CNS of vertebrates. αS is expressed in the neocortex, hippocampus, substantia niagra, thalamus and cerebellum, with the main location within the presynaptic terminals of neurons in both membrane-bound and cytosolic free forms. Presynaptic terminals release chemical messengers, called neurotransmitters, from compartments known as synaptic vesicles. The release of neurotransmitters relays signals between neurons and is critical for normal brain function. αS can be seen in neuroglial cells and melanocytic cells, and is highly expressed in the neuronal mitochondria of the olfactory bulb, hippocampus, striatum and thalamus.

αS aggregates to form insoluble fibrils in pathological conditions characterized by Lewy bodies, such as PD, dementia with Lewy bodies (DLB) and multiple system atrophy (MSA). These disorders are known as synucleinopathies. αS is the primary structural component of Lewy body fibrils. Occasionally, Lewy bodies contain tau protein; however, αS and tau constitute two distinctive subsets of filaments in the same inclusion bodies. αS pathology is also found in both sporadic and familial cases with AD. Thus, one indicator of abnormal αS pathology is the formation of αS aggregates.

At the molecular level, protein misfolding, accumulation, aggregation and subsequently the formation of amyloid deposits are common features in many neurological disorders including AD and PD. Thus neurodegenerative diseases are sometimes referred to as proteinopathies. The existence of a common mechanism suggests that neurodegenerative disorders likely share a common trigger and that the nature of the pathology is determined by the type of the aggregated protein and the localization of the cell affected.

Starting two decades ago with the discoveries of genetic links between αS and PD risk and the identification of aggregated αS as the main protein constituent of Lewy pathology, αS has emerged as the major therapeutic target in PD and related synucleinopathies. Brundin et al., 2017. In recent years, several studies have shown that αS aggregation can also be detected outside the central nervous system, particularly in the ENS of the gastrointestinal tract of PD patients using immunohistochemistry. Further, it has also been reported that αS is a common modifier in motor neuron diseases (Kline et al., 2017), many of which have hallucinations as a related symptom.

Hallucinations affect about 25-40% of patients with PD. Fenelon et al., 2000; and Friedman et al., 2018 (“Hallucinations and delusions are common in Parkinson's disease (PD) whether or not they are associated with dementia. These psychotic symptoms may cause great concern for patients and caregivers. Hallucinations in PD can occur in any sensory modality and sometimes simultaneously. Up to 40% of patients with PD, the majority under treatment with multiple drugs, report these symptoms.”)

Examples of conditions associated with abnormal αS pathology, and/or dopaminergic dysfunction, correlated with hallucinations include, but are not limited to, synucleopathies, neurodiseases, psychological and/or behavior disorders, cerebral and general ischemic disorders, and/or disorders or conditions that are described herein and include. These conditions include, for example, synucleopathies, Parkinson's disease, Alzheimer's disease, dementia with Lewy bodies (DLB), multiple system atrophy (MSA), Huntington's Disease, Multiple Sclerosis (MS), Amyotorphic Lateral Sclerosis (ALS), schizophrenia, Friedreich's ataxia, vascular dementia, spinal muscular atrophy, supranuclear palsy, frontotemporal dementia (FTD), progressive supranuclear palsy, Guadeloupian Parkinsonism, Parkinsonism, spinocerebellar ataxia, autism, stroke, traumatic brain injury, sleep disorders such as REM sleep behavior disorder (RBD), depression, down syndrome, Gaucher's disease (GD), Krabbe's disease (KD), lysosomal conditions affecting glycosphingolipid metabolism, ADHD, agitation, anxiety, delirium, irritability, illusion and delusions, amnesia, apathy, bipolar disorder, disinhibition, aberrant motor and obsessive-compulsive behaviors, addiction, cerebral palsy, epilepsy, major depressive disorder, degenerative processes associated with aging, and dementia of aging.

Several of these conditions are described in more detail below.

I. Neurodegenerative Diseases Associated with Neural Cell Death

i. Synucleinopathies

Synucleinopathies (also called α-Synucleinopathies) are neurodegenerative diseases characterized by the abnormal accumulation of fibrillary aggregates of αS protein in the cytoplasm of selective populations of neurons and glia. These disorders include PD, DLB, pure autonomic failure (PAF), and MSA. Other rare disorders, such as various neuroaxonal dystrophies, also have αS pathologies.

The synucleinopathies have shared features of visual hallucinations, as well as cognitive impairment, parkinsonism, and sleep disorders. Synucleinopathies can sometimes overlap with tauopathies, possibly because of interaction between the synuclein and tau proteins.

αS deposits can affect the cardiac muscle and blood vessels. Almost all people with synucleinopathies have cardiovascular dysfunction, although most are asymptomatic. From chewing to defecation, αS deposits affect every level of gastrointestinal function. Symptoms include upper gastrointestinal tract dysfunction such as delayed gastric emptying or lower gastorintestinal dysfunction, such as constipation and prolonged stool transit time.

Urinary retention, waking at night to urinate, increased urinary frequency and urgency, and over- or underactive bladder are common in people with synucleinopathies. Sexual dysfunction usually appears early in synucleinopathies, and may include erectile dysfunction, and difficulties achieving orgasm or ejaculating.

Patients with neurodegenerative disease, such as PD, AD, LBD, amyloidopathy, etc., frequently experience hallucinations and illusionary perceptions. Burghaus et al., 2012. Synucleinopathies and tauopathies have different risk profiles for hallucinations. In synucleinopathies hallucinations are much more frequent and phenomenology is characterized by visual, short-lived hallucinations, with insight preserved for a long time. In contrast, in tauopathies the hallucinations are more rare and mostly embedded in confusional states with agitation and with poorly defined or rapidly changing paranoia. The occurrence of hallucinations has even been proposed as an exclusion criterion for tauopathies with Parkinsonian features such as progressive supranuclear palsy. To date, treatment remains largely empirical, except the use of clozapine and cholinesterase inhibitors in synucleinopathies, which is evidence-based. The risk of increased neuroleptic sensitivity further restricts the treatment options in patients with Lewy Body Dementia. See also, J. Hinkle and G. Pontone, 2017; D. Collerton and J. Taylor, 2013; and FTD Talk 2015 (“Psychosis is common in the major dementias. It is typical of Dementia with Lewy bodies, very common in Alzheimer's disease and occurs, although to a lesser degree, in vascular dementia.”)

The problem of hallucinations associated with neurodegenerative disease is critical, as the number of people over 60 years is expected to rise from 841 million in 2013 to more than 2 billion in 2050 (United Nations. World population ageing 2013). As populations get older, age-related neurodegenerative diseases such as AD and PD have become more common (Reitz et al., 2011; Reeve et al., 2014). Even for less common neurodegenerative diseases, such as ALS, this trend seems likely (Beghi et al., 2006).

ii. Frontotemporal dementia (FTD)

Frontotemporal dementia (FTD) or frontotemporal degenerations is a clinical term that refers to a group of progressive neurodegenerative disorders that affect the frontal and temporal lobes causing personality change (apathy, disinhibition, loss of insight and emotional control), loss of the ability to recognize the meaning of words and objects, language dysfunction, and global cognitive decline. Unlike AD, which attacks the brain's memory centers, FTD causes atrophy in the part of the brain that controls judgment, behavior and executive function. FTDs have an earlier onset than AD and, at an early stage, do not cause the memory loss and visual-spatial disorientation that are so characteristic of AD. There is an overlap between FTDs, amyotrophic lateral sclerosis (ALS), and atypical parkinsonian syndromes (progressive supranuclear palsy and corticobasal degeneration).

Prior studies have reported the presence of tau and αS inclusions in a case of FTD and progressive aphasia. Yancopoulou et al., 2005. Similarly, a more resent study reported the significant presence of phosphorylated αS-positive structures were also found in oligodendrocytes and the neuropil of FTD patients. Hosokawa et al., 2017.

Hallucinations are observed in about 20-32% or more subjects with FTD (Lindqvist Waldo et al., 2015), and FTD Talk 2015.

iii. Amyotrophic Lateral Sclerosis (ALS)

Amyotrophic lateral sclerosis (ALS), also known as motor neuron disease (MND), or Lou Gehrig's disease, is a specific disease which causes the death of neurons controlling voluntary muscles. ALS is characterized by stiff muscles, muscle twitching, and gradually worsening weakness due to muscles decreasing in size. This results in difficulty speaking, swallowing, and eventually breathing. The cause is not known in 90% to 95% of cases. The remaining 5-10% of cases are genetic. The underlying mechanism involves damage to both upper and lower motor neurons. No cure for ALS is known. The disease can affect people of any age, but usually starts around the age of 60 and in inherited cases around the age of 50. The average survival from onset to death is 2 to 4 years, although about 10% survive longer than 10 years.

Although the degeneration predominantly affects the motor system, cognitive and behavioral symptoms have been described for over a century, and there is evidence that ALS and frontotemporal dementia overlap clinically, radiologically, pathologically, and genetically. Cognitive decline in ALS is characterized by personality change, irritability, obsessions, poor insight, and pervasive deficits in frontal executive tests. This presentation is consistent with the changes to character, social conduct, and executive function in frontotemporal dementia. Phukan et al., 2007.

ALS used to be thought of as a disease purely of the motor system, but more recently a correlation has been identified between ALS and other neurodiseases characterized by hallucinations, such as FTD, schizophrenia, autism, etc. O'Brien et al. 2017. The researchers note that these seemingly different conditions might be biologically related. Disruptions in neural network connectivity have been associated with all of them, implying that could be the common denominator (Li et al., 2015; Wang et al., 2017).

αS pathology has been examined in the brains and spinal cords of patients with ALS/parkinsonism-dementia complex (PDC). Kokubo et al., 2012. This study reported that various types of phosphorylated αS-positive structures were found in all ALS/PDC cases. This is significant as phosphorylated αS is the main component of Lewy bodies (LBs) that are characteristic of PD and DLB.

iv. Huntington's disease (HD)

Huntington's disease (HD) is a progressive brain disorder caused by a defective gene. This disease causes changes in the central area of the brain, which affect movement, mood and thinking skills. HD is a progressive brain disorder caused by a single defective gene on chromosome 4—one of the 23 human chromosomes that carry a person's entire genetic code. This defect is “dominant,” meaning that anyone who inherits it from a parent with Huntington's will eventually develop the disease.

The hallmark symptom of HD is uncontrolled movement of the arms, legs, head, face and upper body. HD also causes a decline in thinking and reasoning skills, including memory, concentration, judgment, and ability to plan and organize. HD symptoms include hallucinations (Correa et al., 2006).

αS also plays a role in the disease pathology of HD. Specifically, recent studies report that αS levels modulate HD in mice. Corrochano et al., February 2012. Similarly, yet another study reported that αS levels affect autophagosome numbers in vivo and modulate HD pathology. Corrochano et al., March 2012.

v. Schizophrenia

Schizophrenia is a chronic progressive disorder that has at its origin structural brain changes in both white and gray matter. It is likely that these changes begin prior to the onset of clinical symptoms in cortical regions, particularly those concerned with language processing. Later, they can be detected by progressive ventricular enlargement. Current magnetic resonance imaging (MRI) technology can provide a valuable tool for detecting early changes in cortical atrophy and anomalous language processing, which may be predictive of who will develop schizophrenia. Hallmark symptoms of schizophrenia include hallucinations. Llorca et al., 2016 (“In schizophrenia patients, hallucinations are hallmark symptoms and auditory ones are described as the more frequent.”).

The duration and strength of the dopaminergic signal are regulated by the dopamine transporter (DAT). Drug addiction and neurodegenerative and neuropsychiatric diseases have all been associated with altered DAT activity. αS, a protein partner of DAT, is implicated in neurodegenerative disease and drug addiction.

A recent study reported that patients with schizophrenia exhibit a decreased expression of αS. Demirel et al. 2017. Specifically, the study reported that schizophrenia subjects exhibited significantly lower serum levels of αS as compared to healthy controls. As serum αS plays a neuromodulator role, this lower amount may result in impaired neuroplasticity in the etiology of schizophrenia, as well as noticeable cognitive impairment which progresses over time.

vi. Multiple Sclerosis

Multiple sclerosis (MS) is a demyelinating disease in which the insulating covers of nerve cells in the brain and spinal cord are damaged. This damage disrupts the ability of parts of the nervous system to communicate, resulting in a range of signs and symptoms, including physical, mental, and sometimes psychiatric problems. Specific symptoms can include double vision, blindness in one eye, muscle weakness, trouble with sensation, or trouble with coordination. MS takes several forms, with new symptoms either occurring in isolated attacks (relapsing forms) or building up over time (progressive forms). Between attacks, symptoms may disappear completely; however, permanent neurological problems often remain, especially as the disease advances. There is no known cure for MS. Life expectancy is on average 5 to 10 years lower than that of an unaffected population. MS is the most common immune-mediated disorder affecting the central nervous system. In 2015, about 2.3 million people were affected globally, and in 2015 about 18,900 people died from MS, up from 12,000 in 1990.

As MS progresses, usually with a series of acute immune attacks and a late-stage steady march of function loss, patients with MS commonly experience fatigue, spasticity, difficulty walking, and cognitive impairment. Rahn et al., 2012. Today physicians recognize that MS affects more than 600,000 people in the United States and more than 2 million people worldwide.

Hallucinations and psychosis are a symptom of MS. Gilberthorpe et al. 2017 (“Psychosis in the context of multiple sclerosis (MS) has previously been reported as a rare occurrence. However, recent epidemiological studies have found prevalence rates of psychosis in MS that are two to three times higher than those in the general population.”) See also, Emin Ozcan et al., 2014.

Abnormal αS pathology is correlated with MS. Specifically, a recent study reported that levels of αS in the Cerebrosinal Fluid (CSF) of MS subjects was significantly lower as compared to healthy controls. Antonelou et al., 2015. Similarly, a more recent study reported the low levels of αS in peripheral tissues are related to clinical relapse in relapse-remitting MS. Mejia et al., 2018.

vii: Various Other Conditions

Progressive supranuclear palsy (PSP), also called Steele-Richardson-Olszewski syndrome, is a brain disorder that causes serious problems with walking, balance and eye movements. The disorder results from deterioration of cells in areas of the brain that control body movement and thinking. There is no known cure for PSP and management is primarily supportive. Visual hallucinations (VH) occur commonly in Parkinson's disease (PD) and dementia with Lewy bodies (DLB) are reported, although less frequently, in other neurodegenerative causes of parkinsonism, such as progressive supranuclear palsy, multiple system atrophy and corticobasal degeneration syndrome. K. Bertram and D. Williams, 2012. PSP is considered a sporadic neurodegenerative disease, one that develops by chance. Build-up of the tau protein in the brain causes cellular damage and thus affects the normal function of neurons. PSP is considered a tauopathy as is AD and other frontotemporal brain disorders. Build-up of the tau protein in PSP is significant, as other researchers have reported that tau and αS appear to promote the fibrillization and solubility of each other in vitro and in vivo. This suggests that interactions between tau and αS form a deleterious feed-forward loop essential for the development and spreading of neurodegeneration. Moussaud et al., 2014.

Vascular dementia, also known as multi-infarct dementia (MID) and vascular cognitive impairment (VCI), is dementia caused by problems in the supply of blood to the brain, typically a series of minor strokes, leading to worsening cognitive decline that occurs step by step. Risk factors for vascular dementia include age, hypertension, smoking, hypercholesterolemia, diabetes mellitus, cardiovascular disease, and cerebrovascular disease. Other risk factors include geographic origin, genetic predisposition, and prior strokes. A characteristic of VCI is cognitive impairment. Vascular dementia is not a single entity, but an umbrella term to describe cognitive decline due to a series of different vessel disorders, frequently seen in combination with other non-vascular changes. These vessel disorders can induce various types of cerebral tissue lesions such as hemorrhage, infarction, hippocampal sclerosis, and white matter lesions. Hallucinations are a symptom or characteristic correlating with vascular dementia.

Spinal muscular atrophy (SMA) is an inherited neuromuscular disorder characterized by loss of motor neurons and progressive muscle wasting, often leading to early death. The disorder is caused by a genetic defect in the SMN1 gene, which encodes SMN, a protein necessary for survival of motor neurons. Lower levels of the protein results in loss of function of neuronal cells in the anterior horn of the spinal cord and subsequent system-wide atrophy of skeletal muscles. It has been reported that significantly lower αS expression were found in tissue samples of SMA patients, suggesting a contribution to the disease pathology. Acsadi et al., 2011. Hallucinations are a symptom or characteristic correlating with SMA.

Friedreich's ataxia (FRDA) is an autosomal recessive inherited disease that causes progressive damage to the nervous system. It manifests in initial symptoms of poor coordination such as gait disturbance; it can also lead to scoliosis, heart disease and diabetes, but does not affect cognitive function. The ataxia of Friedreich's ataxia results from the degeneration of nervous tissue in the spinal cord, in particular sensory neurons essential (through connections with the cerebellum) for directing muscle movement of the arms and legs. The spinal cord becomes thinner and nerve cells lose some of their myelin sheath (the insulating covering on some nerve cells that helps conduct nerve impulses). Recent research reports that cognitive impairment is correlated with FRDA. Dogan et al., 2016. Hallucinations are a symptom or characteristic correlating with FRDA.

### 2. Psychological or Behavior Disorders

i. Sleep Disorders & Sleep Disturbances

Studies have found a correlation between sleep disorders, sleep disturbances and/or sleep fragmentation and the presence of hallucinations, particularly in older adults. Hypnopompic hallucinations refer to hallucinations occurring at the time a person wakes up, and hypnagogic hallucinations refer to those occurring when a person falls asleep. Hypnagogic hallucinations may be caused by Parkinson's disease or schizophrenia. Beginning to hallucinate is among the more common symptoms of sleep deprivation.

It was reported that αS overexpression in mice produces sleep disruptions. McDowell et al., 2014. REM sleep behavior disorder (RBD) is a parasomnia in which individuals with RBD lose the paralysis of muscles (atonia) that is normal during rapid eye movement (REM) sleep, and act out their dreams or have other abnormal movements or vocalizations. Abnormal sleep behaviors may appear decades before any other symptoms, often as an early sign of a synucleinopathy. On autopsy, 94 to 98% of individuals with polysomnography-confirmed RBD are found to have a synucleinopathy—most commonly DLB or PD. Other symptoms of the specific synucleinopathy usually manifest within 15 years of the diagnosis of RBD, but may emerge up to 50 years after RBD diagnosis.

ii. Autism

Autism, or autism spectrum disorder (ASD), refers to a range of conditions characterized by challenges with social skills, repetitive behaviors, speech and nonverbal communication, as well as by unique strengths and differences. There are many types of autism, caused by different combinations of genetic and environmental influences. A recent report noted that hallucinations are unusually common in adults with autism. E. Milne, 2017.

The Centers for Disease Control and Prevention (CDC) estimates autism's prevalence as 1 in 59 children in the United States. This includes 1 in 37 boys and 1 in 151 girls. Around one third of people with autism remain nonverbal, and around one third of people with autism have an intellectual disability. Certain medical and mental health issues frequently accompany autism. They include gastrointestinal (GI) disorders, seizures, sleep disturbances, attention deficit and hyperactivity disorder (ADHD), anxiety and phobias.

A recent brain-tissue study suggests that children affected by autism have a surplus of synapses, or connections between brain cells. The excess is due to a slowdown in the normal pruning process that occurs during brain development. During normal brain development, a burst of synapse formation occurs in infancy. This is particularly pronounced in the cortex, which is central to thought and processing information from the senses. But by late adolescence, pruning eliminates about half of these cortical synapses. In addition, many genes linked to autism are known to affect the development or function of brain synapses. The study also found that the brain cells from individuals with autism were filled with damaged parts and deficient in signs of a normal breakdown pathway called “autophagy.” Tang et al., 2014.

Abnormal αS pathology plays a role in ASD. In particular, a recent study reported that mean plasma αS levels were significantly lower in autism spectrum disorder (ASD) children as compared to healthy controls. W. Sriwimol and P. Limprasert, 2018.

iii. Depression

Depression is frequently associated with abnormal αS pathology, and this condition can also correlate with hallucinations as well as hallucination-related symptoms. Moreover, depressive disorders are associated with problems in multiple cognitive domains including attention (concentration), memory (learning), and decision making (judgment). E. Rubin, 2016.

Some people who have severe clinical depression will also experience hallucinations and delusional thinking, the symptoms of psychosis, referred to as psychotic depression. Individuals with psychotic depression experience the symptoms of a major depressive episode, along with one or more psychotic symptoms, including delusions and/or hallucinations.

Depression is found in 30-40% of all patients with PD, and a postmortem analysis of PD subjects found that a higher prevalence of pathological features were present in depressed compared to non-depressed PD patients. Frisina et al., 2009. This is not surprising as αS is a neuronal protein involved in the regulation of brain serotonin and dopamine levels. Frieling et al., 2008. Further, a correlation between depressive symptoms and αS mRNA expression has been reported in subjects with eating disorders. Id.

3. Ischemic Disorders

The methods and compositions of the invention may also be useful in treating, preventing, and/or delaying the onset or progression of hallucinations and/or a hallucination-related symptom, where the hallucinations are correlated with abnormal α-synuclein (αS) pathology, and/or correlated with dopaminergic dysfunction, where the hallucinations are also correlated with a cerebral or general ischemic disorder.

In some embodiments, the cerebral ischemic disorder comprises cerebral microangiopathy, intrapartal cerebral ischemia, cerebral ischemia during/after cardiac arrest or resuscitation, cerebral ischemia due to intraoperative problems, cerebral ischemia during carotid surgery, chronic cerebral ischemia due to stenosis of blood-supplying arteries to the brain, sinus thrombosis or thrombosis of cerebral veins, cerebral vessel malformations, or diabetic retinopathy.

In some embodiments, the general ischemic disorders comprises high blood pressure, high cholesterol, myocardial infarction, cardiac insufficiency, cardiac failure, congestive heart failure, myocarditis, pericarditis, perimyocarditis, coronary heart disease, angina pectoris, congenital heart disease, shock, ischemia of extremities, stenosis of renal arteries, diabetic retinopathy, thrombosis associated with malaria, artificial heart valves, anemias, hypersplenic syndrome, emphysema, lung fibrosis, or pulmonary edema.

Hallucinations have been correlated with ischemic disorders. Senadim et al., 2017. Studies have also shown a correlation between abnormal αS pathology and ischemic disorders. For example, one study reported that post-stroke induction of αS mediates ischemic brain damage. Kim et al., 2016. Yet another study conducted a comparison of the amount of αS in ischemic stroke and PD subjects, with the results showing that the levels of oligomeric form of αS of red blood cells in ischemic stroke and PD patients were both significant higher than that of healthy controls. Zhao et al., 2016. Finally, another study reported that cerebral ischemic injury leads to a reduction in αS and consequently causes serious brain damage. P. Koh, 2017.

C. Current Treatment of Hallucinations

Current therapies for treating hallucinations caused by a wide variety of diseases generally involve drug therapy. Unfortunately, many of the drugs used in these therapies have significant and deleterious side effects.

Schizophrenia:

Current treatment strategies for hallucinations caused by schizophrenia have poor prognosis. Schizophrenia is a chronic disorder typically affecting young adults. It carries serious social and physical consequences and has a major impact on the individual's productivity and on public health. Positive symptoms such as auditory hallucinations are very common in patients with schizophrenia and indeed one of the cardinal features of the disease. Schizophrenic patients frequently have episodes of disorganized thinking and delusional behavior that require hospitalization. Typical symptoms of schizophrenia include blunting of affect, reduced speech, anhedonia, apathy and anti-social behavior. Additionally, depression, anxiety and pronounced sleep disturbances are commonly associated with schizophrenia.

Although, anti-psychotic agents have been shown to be of some benefit in reducing hallucinations and other psychotic features during acute episodes, they are of little value in preventing or reducing the frequency of subsequent hallucinations. Moreover, the side effects of the anti-psychotic medications result in poor patient compliance to using these medications as prescribed. These side effects include extrapyramidal symptoms such as dystonia, akathisia and tardive dyskinesia, weight gain, sedation and metabolic effects, and as a result an overall increase in morbidity. Second generation anti-psychotics tend to control the negative symptoms better than first generation anti-psychotics but are also associated with increased metabolic abnormalities. Further, the effectiveness of current drugs for the treatment of schizophrenia may occur in only about 50% of patients. Poor responses are associated with poor compliance with medication, exacerbation of symptoms and increased risk of hospitalization with resultant higher costs of treatment. In the Clinical Antipsychotic Trials of Intervention (CATIE) study, which compared the relative effectiveness of perphenazine, olanzapine, quetiapine, risperidone and ziprasidone in 1493 patients over an 18 month period, over 1100 patients or 75% of the total withdrew from the study, either because of intolerable side effects or because of inefficacy.

Thus, the ideal medication for treating hallucination aims to improve anhedonia, apathy, depression, and anti-social behavior associated with schizophrenia. In addition, the medication should be tolerable, should not cause exacerbation of symptoms, should not lead to extrapyramidal side effects such as akathisia, dyskinesia and tardive dyskinesia, or to metabolic abnormalities such as diabetes, weight gain, high cholesterol levels, and should not affect the QT interval of the EKG.

Parkinson's Disease:

Current treatments for Parkinson's disease (PD) associated hallucinations are also unsatisfactory. The first measure to treat PD related hallucinations is to discontinue the use of anticholinergics, selegiline, amantadine, dopamine agonists, COMT inhibitors and even levodopa/carbidopa as a last resort. However, discontinuation of these PD treatments may significantly worsen the motor symptoms of the condition.

Hallucinations are a common nonmotor feature of PD, being present in up to 30%-40% of patients with late stage disease. Hallucinations and cognitive dysfunction are common causes of institutionalization in this patient population and significantly increase the cost of care. Use of the older antipsychotic drugs frequently leads to worsening of motor symptoms. Newer antipsychotics such as clozapine, risperidone, olanzapine, aripiprazole and quetiapine have broadened therapeutic options and all are used off-label to treat PD hallucinations. Although clozapine has proven efficacy, it is often avoided due to its potential for drug-induced agranulocytosis and the need for regular monitoring of blood tests. In open label trials quetiapine has similar efficacy to clozapine but the results of several randomized, controlled trials (RCTs) have been disappointing. Furthermore, many of these compounds also lead to worsening of motor symptoms of the disease. Pimavanserin (Nuplazid, Acadia Pharmaceuticals, Inc.) was the first compound approved by the FDA for the treatment of PD related hallucinations, although the efficacy is limited both in the degree of reduction of hallucinations (only a 3 point improvement over placebo on the SCAD-PD questionnaire) and the % of patients who benefited at all, and furthermore the label contains a black box warning about an 11% increased mortality, largely caused by QT prolongation on EKG causing cardiac arrhythmias and death. Other than the cardiac issues, treatment with Pimavanserin can also cause the patient to exhibit a state of confusion and worsening hallucinations. The ideal medication for treating hallucinations caused by PD would aim to avoid the above side effects.

The present inventors have surprisingly discovered that aminosterols, such as squalamine, aminosterol 1436, and derivatives thereof, when administered orally or nasally, can treat or prevent hallucinations in a subject in need thereof and avoid most of the side effects of the conventional hallucination treatment strategies.

D. Summary of Experimental Results

This disclosure provides examples of treatment of hallucinations using aminosterols. In Example 4, a patient suffering from PD and hallucinations was treated starting with 75 mg of squalamine daily. As the dose was increased, the patient reported that he was hallucinating less frequently. When the daily dose of squalamine was increased to 125 mg, the hallucinations disappeared completely. The dose was increased to 175 mg, and maintained at 175 mg per day for another week or two, before discontinued. The patient remained hallucination-free for another 30 days after discontinuation of the treatment. Examples 2 and 3 are directed to similar treatment in similar patients with similar results. The patients of Example 4 also suffered from REM-behavioral disorder (RBD), and saw RBD symptoms improve with the squalamine treatment.

As described in Example 4, a study was conducted in patients with Parkinson's disease (PD). Example 4 differs from Examples 1-3 in that it involves monitoring a symptom of the hallucination while escalating the aminosterol dose, and determining a fixed dose of an aminosterol or a salt or derivative thereof to administer based on the improvement of the hallucination symptom being monitored. Hallucination symptoms monitored include, but are not limited to, auditory hallucination, visual hallucination, cognitive impairment and constipation. Additional hallucination symptoms that can be utilized in the methods of the invention are described herein.

PD is a progressive neurodegenerative disorder caused by accumulation of the protein α-synuclein (αS) within the enteric nervous system (ENS), autonomic nerves and brain. While the study described in Example 4 assessed patients with PD, many symptoms assessed and contemplated to be resolved by amino sterol treatment are not restored by the replacement of dopamine. Examples of such symptoms include, but are not limited to, constipation, disturbances in sleep architecture, cognitive impairment or dysfunction, hallucinations, REM behavior disorder (RBD), and depression. Other relevant symptoms are described herein. All of all of these symptoms result from impaired function of neural pathways not restored by replacement of dopamine in PD subjects.

A strategy that targets neurotoxic aggregates of αS in the gastrointestinal tract represents a novel approach to the treatment of PD and other symptoms associated therewith including hallucinations. Treatment and conditions described herein that may restore the function of enteric nerve cells and prevent retrograde trafficking to the brain. Such actions may potentially slow progression of the disease in addition to restoring gastrointestinal function.

Not to be bound by theory, it is believed that amino sterols target neurotoxic aggregates of αS in the gastrointestinal tract, and restore function of the enteric nerve cells. The now-functional enteric nerve cells prevent retrograde trafficking of proteins, such as alpha-synuclein, to the brain. In addition to restoring gastrointestinal function, this effect is believed to slow and possibly reverse PD associated symptoms, including hallucinations.

The methods and compositions disclosed herein permit exerting pharmacological control over the ENS in a manner that is without precedent in the literature. A strategy that targets neurotoxic aggregates of αS in the GI tract represents a novel approach to the treatment of hallucinations and/or related symptoms correlated with abnormal αS pathology and/or correlated with dysfunctional DA neurotransmission/dopaminergic dysfunction. Treatment and conditions described herein may restore the function of enteric nerve cells and prevent retrograde trafficking to the brain. Such actions may potentially slow progression and/or onset of hallucinations and/or related symptoms and/or the underlying disease or condition.

Constipation serves as symptom of many neurodiseases such as PD. Not to be bound by theory, based on the data described herein, it is believed that amino sterols improve bowel function by acting locally on the gastrointestinal tract (as supported by the oral bioavailability <0.3%). An orally administered amino sterol such as squalamine, the active ion of ENT-01, stimulates gastro-intestinal motility in mice with constipation due to overexpression of human αS (West et al, manuscript in preparation). Perfusion of an aminosterol such as squalamine through the lumen of an isolated segment of bowel from the PD mouse model results in excitation of IPANs (intrinsic primary afferent neuron), the major sensory neurons of the ENS that communicate with the myenteric plexus, increasing the frequency of propulsive peristaltic contractions and augmenting neural signals projecting to the afferent arm of the vagus.

Systemic absorption of the aminosterol following oral administration was negligible both in this study and in prior studies involving mice, rats and dogs. Prior studies demonstrated that intravenous administration of squalamine was not associated with increased gastrointestinal motility, despite reaching systemic blood levels one thousand-fold greater than that achieved by orally administered squalamine. These data suggest that the effect is mediated by local action in the GI tract. The topical action would also explain why adverse events were largely confined to the gastrointestinal tract.

Several exploratory endpoints were incorporated into the trial described in Example 4 to evaluate the impact of an amino sterol on neurologic symptoms associated with a neurodisease such as PD, including hallucinations. Following aminosterol treatment, the Unified Parkinson's Disease Rating Scale (UPDRS) score, a global assessment of motor and non-motor symptoms, showed significant improvement. Improvement was also seen in the motor component. The improvement in the motor component is unlikely to be due to improved gastric motility and increased absorption of dopaminergic medications, since improvement persisted during the 2-week wash-out period, i.e., in the absence of study drug (Table 12).

Improvements were also seen in hallucinations, cognitive function (MMSE scores), REM-behavior disorder (RBD) and sleep. Six of the patients enrolled had daily hallucinations or delusions and these improved or disappeared during treatment in five. In one patient the hallucinations disappeared at 100 mg, despite not having reached the colonic prokinetic dose (e.g., fixed escalated aminosterol dose) of 175 mg for this particular patient. The patient remained free of hallucinations for 1 month following cessation of dosing. RBD and total sleep time also improved progressively in a dose-dependent manner.

Interestingly, most indices related to bowel function returned to baseline value by the end of the 2-week wash-out period while improvement in the CNS symptoms persisted. The rapid improvement in certain CNS symptoms is consistent with a mechanism whereby nerve impulses initiated from the ENS following aminosterol administration augment afferent neural signaling to the CNS. This may stimulate the clearance of αS aggregates within the afferent neurons themselves as well as the secondary and tertiary neurons projecting rostrally within the CNS, since it is known that neural stimulation is accompanied by increased neuronal autophagic activity (Shehata et al. 2012). It is believed that after cessation of amino sterol administration, the neurons of the CNS gradually re-accumulate an αS burden either locally or via trafficking from αS re-aggregation within the gut.

Disturbance of the circadian rhythm has been described in neurodiseases such as PD both clinically and in animal models and might plays a role in the abnormal sleep architecture, dementia, mood and autonomic dysfunction associated with neurodiseases such as PD (Breen et al. 2014; Videnovic et al. 2017; Antonio-Rubio et al. 2015; Madrid-Navarro et al. 2018). Circadian rhythm was monitored through the use of a temperature sensor that continuously captured wrist skin temperature (Sarabia et al. 2008), an objective measure of the autonomic regulation of vascular perfusion (Videnovic et al. 2017). Circadian cycles of wrist skin temperature have been shown to correlate with sleep wake cycles, reflecting the impact of nocturnal heat dissipation from the skin on the decrease in core temperature and the onset of sleep (Sarabia et al. 2008; Ortiz-Tuleda et al. 2014). Oral administration of ENT-01 had a significant positive impact on the circadian rhythm of skin temperature in the 12 patients with evaluable data. Not to be bound by theory, it is believed that amino sterols could be affecting neuronal circuits involving the master clock (the suprachiasmatic nucleus) and its autonomic projections and opens the possibility of therapeutic correction of circadian dysfunction.

Most surprisingly, as described in Example 4, it was discovered that aminosterol dosing is patient specific, as the dose is likely related to the extent of neuronal damage, with greater neuronal damage correlating with the need for a higher aminosterol dose to obtain a desired therapeutic result (e.g., treating hallucinations). This was not known prior to the present invention. Thus, one aspect of the present invention is directed to methods of treating, preventing, and/or slowing the onset or progression of hallucinations and/or a hallucination related symptom in a subject in need, where the method comprises determining an effective therapeutic aminosterol dose for the subject. The method comprises a first step of identifying a hallucination-related symptom to be evaluated for determining the effective therapeutic aminosterol dose for the subject. As described in greater detail herein, in one embodiment aminosterol dosing can range from about 0.01 to about 500 mg/day, with dosage determination described in more detail below.

Low bioavailability: As described in Example 4, in preclinical studies, squalamine (ENT-01) exhibited an oral bioavailability of about 0.1% in both rats and dogs. In Stage 1 of the phase 2 study, oral dosing up to 200 mg (114 mg/m²) yielded an approximate oral bioavailability of about 0.1%, based on a comparison of a pharmacokinetic data of the oral dosing and the pharmacokinetic data measured during prior phase 1 studies of IV administration of squalamine. Thus, in one embodiment of the invention, aminosterol dosing, either oral or intranasal, results in a bioavailability of less than about 3%, less than about 2.5%, less than about 2%, less than about 1.5%, less than about 1%, less than about 0.9%, less than about 0.8%, less than about 0.7%, less than about 0.6%, less than about 0.5%, less than about 0.4%, less than about 0.3%, less than about 0.2%, or about 0.1% or less.

In addition, it was also surprisingly discovered that the starting dose of the aminosterol or a salt or derivative thereof is dependent upon the severity of hallucinations and/or a hallucination related symptom. Specifically, if the hallucinations and/or a hallucination related symptom is severe, then the starting aminosterol dose, prior to dose escalation, should be higher than if the hallucinations and/or a hallucination related symptom is moderate. “Severe” hallucinations can be determined by a clinical scale or tool appropriate for measuring the identified hallucination and/or a hallucination related symptom.

One impact of the present invention is that recognizing that an aminosterol dose useful in treating hallucinations and/or a hallucination related symptoms is patient specific can prevent the use of incorrect amino sterol doses for patients. This is a significant discovery, as if a subject is put on an aminosterol dose that is too high, then resultant nausea, vomiting, and abdominal discomfort can result in the patient going off the drug, with the hallucinations and/or a hallucination related symptoms remaining untreated. Similarly, if a subject is put on an aminosterol dose that is too low, then the hallucinations and/or a hallucination related symptoms will not be successfully treated. Prior to the present invention, there was no recognition that therapeutically effective aminosterol doses had no relation to the sex, age, weight, ethnicity, or other similar patient characteristics. This is unexpected, as it is contrary to dosing strategies for almost all other medications.

### II. Methods of Treatment

The present application provides methods for the treatment and prevention of hallucinations using aminosterols. Thus, in one aspect, a method of treating, preventing and/or slowing the onset or progression of hallucinations and/or a related symptom in a subject in need is provided, the method comprising selecting a subject suffering from or potentially susceptible to hallucinations; and administering to the subject a therapeutically effective amount of at least one aminosterol, or a salt or derivative thereof.

Selecting a subject suffering from hallucinations may comprise selecting a subject with a threshold score qualifying as suffering from hallucinations, as measured by a medically recognized technique selected from the group consisting of Chicago Hallucination Assessment Tool (CHAT), The Psychotic Symptom Rating Scales (PSYRATS), Auditory Hallucinations Rating Scale (AHRS), Hamilton Program for Schizophrenia Voices Questionnaire (HPSVQ), Characteristics of Auditory Hallucinations Questionnaire (CAHQ), Mental Health Research Institute Unusual Perception Schedule (MUPS), positive and negative syndrome scale (PANSS), scale for the assessment of positive symptoms (SAPS), Launay-Slade hallucinations scale (LSHS), the Cardiff anomalous perceptions scale (CAPS), and structured interview for assessing perceptual anomalies (SIAPA).

In some embodiments, the therapeutically effective amount of the at least one amino sterol, or a salt or derivative thereof comprises about 0.001 to about 500 mg per day. In some embodiments, the therapeutically effective amount of the at least one amino sterol, or a salt or derivative thereof comprises about 0.001 to about 500 mg per day, about 0.001 to about 375 mg per day, about 0.001 to about 250 mg per day, or about 0.001 to about 125 mg per day. In some embodiments, the therapeutically effective amount of the at least one amino sterol, or a salt or derivative thereof comprises about 0.001 to about 375 mg per day. In some embodiments, the therapeutically effective amount of the at least one amino sterol, or a salt or derivative thereof comprises about 0.001 to about 250 mg per day. In some embodiments, the therapeutically effective amount of the at least one amino sterol, or a salt or derivative thereof comprises about 0.001 to about 125 mg per day.

In some embodiments, the administration comprises nasal administration and the therapeutically effective amount of the at least one amino sterol, or a salt or derivative thereof comprises about 0.001 to about 6 mg per day. In some embodiments, the administration comprises nasal administration and the therapeutically effective amount of the at least one amino sterol, or a salt or derivative thereof comprises about 0.001 to about 4 mg per day. In some embodiments, the administration comprises nasal administration and the therapeutically effective amount of the at least one amino sterol, or a salt or derivative thereof comprises about 0.001 to about 2 mg per day. In some embodiments, the administration comprises nasal administration and the therapeutically effective amount of the at least one amino sterol, or a salt or derivative thereof comprises about 0.001 to about 1 mg per day.

In some embodiments, the administration comprises oral administration and the therapeutically effective amount of the at least one amino sterol, or a salt or derivative thereof comprises about 1 to about 300 mg per day. In some embodiments, the administration comprises oral administration and the therapeutically effective amount of the at least one amino sterol, or a salt or derivative thereof comprises about 25 to about 300 mg per day. In some embodiments, the administration comprises oral administration and the therapeutically effective amount of the at least one amino sterol, or a salt or derivative thereof comprises about 75 to about 300 mg per day. In some embodiments, the administration comprises oral administration and the therapeutically effective amount of the at least one amino sterol, or a salt or derivative thereof comprises about 100 to about 300 mg per day. In some embodiments, the administration comprises oral administration and the therapeutically effective amount of the at least one amino sterol, or a salt or derivative thereof comprises about 150 to about 300 mg per day. In some embodiments, the administration comprises oral administration and the therapeutically effective amount of the at least one amino sterol, or a salt or derivative thereof comprises about 200 to about 300 mg per day.

The method of claim 1, wherein the therapeutically effective amount of the at least one amino sterol, or a salt or derivative thereof comprises about 0.1 to about 20 mg/kg body weight of the subject. The method of claim 1, wherein the therapeutically effective amount of the at least one amino sterol, or a salt or derivative thereof comprises about 0.1 to about 5 mg/kg body weight of the subject. The method of claim 1, wherein the therapeutically effective amount of the at least one amino sterol, or a salt or derivative thereof comprises about 5 to about 10 mg/kg body weight of the subject. The method of claim 1, wherein the therapeutically effective amount of the at least one amino sterol, or a salt or derivative thereof comprises about 10 to about 15 mg/kg body weight of the subject. The method of claim 1, wherein the therapeutically effective amount of the at least one amino sterol, or a salt or derivative thereof comprises about 15 to about 20 mg/kg body weight of the subject.

### III. Methods of Determining a “Fixed Dose” of Aminosterol

In one embodiment, the present application relates to the surprising discovery of a method to determine a “fixed dose” of an amino sterol composition useful for treating, preventing and/or slowing the onset or progression of hallucinations and/or a hallucination related symptom in a subject that is not age, size, or weight dependent but rather is individually calibrated. In one embodiment, the hallucination is correlated with abnormal αS pathology and/or dysfunctional DA neurotransmission and/or dopaminergic dysfunction. The “fixed dose” obtained through this method yields highly effective results in treating, preventing and/or slowing the onset or progression of hallucinations and/or a hallucination-related symptom.

A. “Fixed Aminosterol Dose”

A “fixed aminosterol dose,” also referred to herein as a “fixed escalated aminosterol dose,” which will be therapeutically effective, is determined for each subject by establishing a starting dose of an amino sterol composition and a threshold for improvement of hallucinations and/or a hallucination-related symptom. Following determining a starting dose of an aminosterol or a salt or derivative thereof for a particular subject, the aminosterol dose is then progressively escalated by a consistent amount over consistent time intervals until the desired improvement in hallucinations and/or a hallucination-related symptom is achieved; this aminosterol dosage is the “fixed escalated aminosterol dosage” for that particular subject for that particular hallucination-related symptom.

In exemplary embodiments, an orally administered aminosterol dose is escalated every about 3 to about 5 days by about 25 mg until the desired improvement is reached. Symptoms evaluated, along with tools for measuring symptom improvement, are specifically described below.

This therapeutically effective “fixed dose” is then maintained throughout treatment and/or prevention. Thus, even if the subject goes “off drug” and ceases taking the aminosterol composition, the same “fixed dose” is taken with no ramp up period following re-initiation of aminosterol treatment for hallucinations and/or a hallucination-related symptom.

Not to be bound by theory, it is believed that the aminosterol dose is dependent on the severity of nerve damage relating to hallucinations and/or a hallucination-related symptom, e.g. the dose may be related to the extent of nervous system damage in the subject's gut.

The amino sterol can be administered via any pharmaceutically acceptable means, such as by injection (e.g., IM, IV, or IP), oral, pulmonary, intranasal, etc. Preferably, the aminosterol is administered orally, intranasally, or a combination thereof.

Oral dosage of an amino sterol can range from about 1 to about 500 mg/day, or any amount in-between these two values. Other exemplary dosages of orally administered aminosterols include, but are not limited to, about 5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, about 300, about 305, about 310, about 315, about 320, about 325, about 330, about 335, about 340, about 345, about 350, about 355, about 360, about 365, about 370, about 375, about 380, about 385, about 390, about 395, about 400, about 405, about 410, about 415, about 420, about 425, about 430, about 435, about 440, about 445, about 450, about 455, about 460, about 465, about 470, about 475, about 480, about 485, about 490, about 495, or about 500 mg/day.

Intranasal dosages of an amino sterol are much lower than oral dosages of an amino sterol. Examples of such intranasal amino sterol low dosages include, but are not limited to, about 0.001 to about 6 mg/day, or any amount in-between these two values. For example, the low dosage of an intranasally administered aminosterol can be about 0.001, about 0.005, about 0.01, about 0.02, about 0.03, about 0.04, about 0.05, about 0.06, about 0.07, about 0.08, about 0.09, about 0.1, about 0.2, about 0.3, about 0.4, about 0.5, about 0.6, about 0.7, about 0.8, about 0.9, about 1, about 1.1, about 1.2, about 1.3, about 1.4, about 1.5, about 1.6, about 1.7, about 1.8, about 1.9, about 2, about 2.1, about 2.2, about 2.3, about 2.4, about 2.5, about 2.6, about 2.7, about 2.8, about 2.9, about 3, about 3.1, about 3.2, about 3.3, about 3.4, about 3.5, about 3.6, about 3.7, about 3.8, about 3.9, about 4, about 4.1, about 4.2, about 4.3, about 4.4, about 4.5, about 4.6, about 4.7, about 4.8, about 4.9, about 5, about 5.1, about 5.2, about 5.3, about 5.4, about 5.5, about 5.6, about 5.7, about 5.8, about 5.9, or about 6 mg/day.

For intranasal (IN) administration, it is contemplated that the aminosterol dosage may be selected such that it would not provide any pharmacological effect if administered by any other route and, in addition, does not result in negative effects. For example, Aminosterol 1436 is known to have the pharmacological effects of a reduction in food intake and weight loss. Therefore, in the IN methods of the invention, if the aminosterol is Aminosterol 1436 or a salt or derivative thereof, then if the IN Amino sterol 1436 dosage is administered via another route, such as oral, IP, or IV, then the Aminosterol 1436 dosage will not result in a noticeable reduction in food intake or noticeable weight loss. Similarly, squalamine is known to produce the pharmacological effects of nausea, vomiting and/or reduced blood pressure. Thus, in the IN methods of the invention, if the aminosterol is squalamine or a salt or derivative thereof, then if the IN squalamine dosage is administered via another route, such as oral, IP, or IV, then the squalamine dosage will not result in noticeable nausea, vomiting, and/or a reduction in blood pressure.

Dose Escalation:

When determining a “fixed aminosterol dosage” for a particular subject, a subject is started at a lower dose and then the dose is escalated until a positive result is observed for hallucinations and/or a hallucination-related symptom. For example, determination of the fixed aminosterol dosage for treating hallucinations and/or a hallucination-related symptoms is shown in Example 4. Aminosterol doses can also be de-escalated (reduced) if any given aminosterol dose induces a persistent undesirable side effect, such as diarrhea, vomiting, or nausea.

The starting aminosterol dose is dependent on the severity of the symptom—e.g. for a subject experiencing severe hallunications based on a baseline score of a clinical test or tool that correlates with an assessment of severe hallucinations, the starting oral aminosterol dose can be about 150 mg/day or greater. In contrast, for a subject having mild or moderate hallucinations based on a baseline score of a clinical test or tool that correlates with an assessment of mild or moderate hallucinations, the starting aminosterol dose can be about 75 mg/day or less. Thus, as an example, a subject experiencing mild or moderate hallucinations can be started at an aminosterol dosage of about 75 mg/day, whereas a subject experiencing severe hallucinations can be started at an aminosterol dosage of about 150 mg/day.

In other embodiments, a subject experiencing mild or moderate hallucination symptoms can be started at an oral aminosterol dosage of from about 10 mg/day to about 75 mg/day, or any amount in-between these values. The mild or moderate symptom may be mild or moderate hallucinations based on a baseline score on a clinical test or tool that correlates with an assessment of mild or moderate hallucinations. For example, starting oral amino sterol dosage for patients with moderate or mild hallucinations can be about 1, about 5, about 10, about 15, about 20, about 25, about 30, about 35, about 40, about 45, about 50, about 55, about 60, about 65, about 70, up to less than or equal to about 75 mg/day. A fixed escalated oral aminosterol dose for a patient with mild or moderate hallucinations is likely to range from about 5 mg up to about 350 mg/day, or any amount in-between these two values as described herein. In some embodiments, an oral fixed amino sterol dose, following dose escalation, is from about 50 to about 300 mg/daily, or from about 75 to about 275 mg/daily.

In yet further embodiments, when the subject is experiencing severe hallucinations or hallucination-related symptoms, as for example defined by a baseline score on a clinical test or tool that correlates with severe hallucinations, the subject can be started at an oral aminosterol dosage ranging from about 75 to about 300 mg/day, or any amount in-between these two values. In other embodiments, the starting oral aminosterol dosage for patients with severe hallucinations or hallucination-related symptoms can be, for example, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, or about 300 mg/day. A “fixed escalated” oral aminosterol dose for a patient with severe hallucinations or hallucination-related symptoms is likely to range from about 75 mg up to about 500 mg/day.

Starting IN aminosterol dosages prior to dose escalation can be, for example, about 0.001 mg to about 3 mg/day, or any amount in-between these two values. For example, the starting aminosterol dosage for IN administration, prior to dose escalation, can be, for example, about 0.001, about 0.005, about 0.01, about 0.02, about 0.03, about 0.05, about 0.06, about 0.07, about 0.08, about 0.09, about 0.1, about 0.15, about 0.2, about 0.25, about 0.3, about 0.35, about 0.4, about 0.45, about 0.5, about 0.55, about 0.6, about 0.65, about 0.7, about 0.75, about 0.8, about 0.85, about 0.9, about 1.0, about 1.1, about 1.25, about 1.3, about 1.4, about 1.5, about 1.6, about 1.7, about 1.75, about 1.8, about 1.9, about 2.0, about 2.1, about 2.25, about 2.3, about 2.4, about 2.5, about 2.6, about 2.7, about 2.75, about 2.8, about 2.9, or about 3 mg/day.

In exemplary embodiments, the fixed dose of the aminosterol is given periodically as needed. For example, the fixed amino sterol dose can be given once per day. The aminosterol dose can also be given every other day, 2, 3, 4, 5 or 6× per week, once/week, or 2×/week. In another embodiment, the aminosterol dose can be given every other week, or it can be given for a few weeks, followed by skipping a few weeks (as the effects persist following treatment), followed by restarting aminosterol treatment.

When calculating a fixed escalated aminosterol dose, the dose can be escalated following any suitable time period. In one embodiment, the aminosterol dose is escalated every about 3 to about 7 days by about a defined amount until a desired improvement is reached. In one embodiment, the aminosterol dose is escalated every about 3 to 5 days until a desired improvement is reached. For example, in some embodiments, the improvement in the hallucination-related symptom is measured using a clinical scale or tool and the improvement is about 3%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In other embodiments, the amino sterol dose can be escalated every about 1, about 2, about 3, about 4, about 5, about 6, about 7, about 8, about 9, about 10, about 11, about 12, about 13, or about 14 days. In other embodiments, the aminosterol dose can be escalated about 1×/week, about 2×/week, about every other week, or about 1×/month.

During dose escalation, the aminosterol dosage can be increased by a defined amount. For example, when the aminosterol is administered orally, the dose can be escalated in increments of about 5, about 10, about 15, about 20, about 25, about 30, about 35, about 40, about 45, or by about 50 mg. When the aminosterol is administered intranasally, then the dosage can be increased in increments of about, for example, about 0.1, about 0.2, about 0.25, about 0.3, about 0.35, about 0.4, about 0.45, about 0.5, about 0.55, about 0.6, about 0.65, about 0.7, about 0.75, about 0.8, about 0.85, about 0.9, about 0.95, about 1, about 1.1, about 1.2, about 1.3, about 1.4, about 1.5, about 1.6, about 1.7, about 1.8, about 1.9, or about 2 mg.

In exemplary embodiments, an orally administered aminosterol dose is escalated every about 3 to about 5 days by about 25 mg until an improvement of hallucinations or hallucination-related symptom is observed. The improvement of the hallucination related symptom may be measured using a clinical scale or tool, and the improvement is about 3%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In another embodiment, a fixed dose of an amino sterol can be varied plus or minus a defined amount to enable a modest reduction in a dose to eliminate adverse events, or a modest increase in a dose if clinical results suggest this is desirable—e.g., no or minimal adverse events and potential increased efficacy with a modest increase in dose. For example, in one embodiment a fixed amino sterol dose can be increased or decreased by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20%.

B. Hallucinations and Hallucination-Related Symptoms to be Evaluated

The “fixed” dose of an aminosterol or a salt or derivative thereof is determined based upon the effect an escalated aminosterol dose has, over a period of time, on hallucinations or a hallucination-related symptom. Measurable hallucination-related symptoms that can be evaluated include, for example: (a) a symptom from the Chicago Hallucination Assessment Tool (CHAT) selected from the group consisting of hallucination frequency, duration, sensory intensity, complexity, controllability, amount of negative content, degree of negative content, frequency of negative emotion associated with hallucination, and intensity of emotional impact, and chronicity; (b) a symptom from the Mental Health Research Institute Unusual Perceptions Schedule (MUPS) selected from the group consisting of onset and course, number, volume, tone, and location; (c) auditory hallucination; (d) tactile hallucination; (e) visual hallucination; (f) olfactory hallucination; (g) gustatory hallucination; (h) delusions; (i) proprioceptive hallucination; (j) equilibrioceptive hallucination; (k) nociceptive hallucination; (l) thermoceptive hallucination; (m) chronoceptive hallucination; (n) non-auditory command hallucination; (o) psychosis; (p) peduncular hallucinosis; (p) delirium; (r) dementia; (s) neurodegenerative disease; (t) neurodegeneration; (u) epilepsy; (v) seizures; (w) migraines; (x) cognitive impairment, e.g., as determined by an IQ score or by a memory or cognitive function test; (y) constipation; (z) depression; (aa) sleep problem, sleep disorder, or sleep disturbance; or (bb) gastrointestinal disorders. The symptoms can be measured using a clinically recognized scale or tool, as detailed herein.

The disclosed methods comprising administering a therapeutically effective amount of at least one aminosterol can be used to treat, prevent and/or slow the onset or progression of hallucinations and/or hallucination-related symptoms. For the purposes of this disclosure, a subject is treated if one or more beneficial or desired results, including desirable clinical results, are obtained. For example, beneficial or desired clinical results include, but are not limited to that the subject experiences a reduction in the number of hallucinations, a reduction in the severity of the hallucinations, or becomes hallucination free.

In an exemplary embodiment of the invention, a decrease in the number of hallucinations, or severity of hallucinations, is defined as a reduction in occurrence or severity of hallucinations over a defined period of time by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%. In one embodiment, the subject is hallucination free. The “defined period of time” can be, for example, about 12 hours, about 24 hours, about 2 days, about 3 days, about 4 days, about 5 days, about 6 days, about one week; about 2, about 3, or about 4 weeks; about 1, about 2, about 3, about 4, about 5, about 6, about 7, about 8, about 9, about 10, about 11, or about 12 months, or about 1 year or longer.

In one aspect, the amino sterol or a salt or derivative thereof is administered to a subject suffering from hallucinations caused by a psychiatric disorder, wherein the aminosterol reverses the dysfunction of the psychiatric disorder and treats the hallucination. In some embodiments, the psychiatric disorder treated by the presently disclosed methods is selected from the group consisting of bipolar disorder, borderline personality disorder, depression (mixed), dissociative identity disorder, generalized anxiety disorder, major depression, obsessive compulsive disorder, post-traumatic stress disorder, psychosis (NOS), schizoaffective disorder, and schizophrenia.

In another aspect, the aminosterol or its derivatives is administered to a subject suffering from hallucinations caused by a neurological disorder, wherein the aminosterol reverses the dysfunction of the neurological disorder and treats the hallucination. In some embodiments, the neurological disorder is a brain tumor. In some embodiments, the neurological disorder is the result of a focal brain lesion. In a further embodiment, the focal brain lesion is occipital lobe lesions or temporal lobe lesions. In a yet further embodiment, the temporal lobe lesion is selected from the group consisting of lesions of the uncinate gyrus, cerebral peduncles, and substantia nigra. In another embodiment, the neurological disorder is the result of a diffuse involvement of the cerebral cortex. In a further embodiment, the diffuse involvement of the cerebral cortex is caused by a viral infectious disease selected from the group consisting of acute metabolic encephalopathies, encephalitis, and meningitis, or by a cerebral vasculitis condition such as autoimmune disorders, bacterial or viral infection, or systemic vasculitis.

In another aspect, the aminosterol or its derivative is administered to a subject suffering from hallucinations caused by a neurodegenerative disorder, wherein the aminosterol reverses the dysfunction of the neurodegenerative disorder and treats the hallucination. In some embodiment, the neurodegenerative disorder is selected from the group consisting of synucleopathies, Parkinson's disease, Alzheimer's disease, dementia with Lewy bodies (DLB), multiple system atrophy (MSA), Huntington's Disease, Multiple Sclerosis (MS), Amyotorphic Lateral Sclerosis (ALS), schizophrenia, Friedreich's ataxia, vascular dementia, spinal muscular atrophy, supranuclear palsy, fronto temperal dementia (FTD), progressive supranuclear palsy, Guadeloupian Parkinsonism, Parkinsonism, spinocerebellar ataxia, autism, stroke, traumatic brain injury, sleep disorders such as REM sleep behavior disorder (RBD), depression, down syndrome, Gaucher's disease (GD), Krabbe's disease (KD), lysosomal conditions affecting glycosphingolipid metabolism, ADHD, agitation, anxiety, delirium, irritability, illusion and delusions, amnesia, apathy, bipolar disorder, disinhibition, aberrant motor and obsessive—compulsive behaviors, addiction, cerebral palsy, epilepsy, major depressive disorder, degenerative processes associated with aging, and dementia of aging. In a particular embodiment, the aminosterol reverses the dysfunction of the neurodegenerative disorder and treats the hallucination caused by the neurodegenerative disorder.

In another aspect, the aminosterol or its derivative is administered to a subject suffering from hallucinations caused by a sensory loss, wherein the aminosterol reverses the dysfunction of the sensory loss and treats the hallucination. In some embodiments, the sensory loss is visual. In some embodiments, the sensory loss is auditory. In some embodiments, the sensory loss is gustatory. In some embodiments, the sensory loss is tactile. In some embodiments, the sensory loss is olfactory.

In another aspect, the aminosterol or its derivative is administered to a subject suffering from hallucinations caused by dysfunction of the enteric nervous system, wherein the aminosterol reverses the dysfunction of the enteric nervous system and treats the hallucination.

Other symptoms that can be used as an endpoint to determine aminosterol dosage for a patient's fixed escalated amino sterol dosage are described herein and include, but are not limited to, (a) a symptom from the Chicago Hallucination Assessment Tool (CHAT) selected from the group consisting of hallucination frequency, duration, sensory intensity, complexity, controllability, amount of negative content, degree of negative content, frequency of negative emotion associated with hallucination, and intensity of emotional impact, and chronicity; (b) a symptom from the Mental Health Research Institute Unusual Perceptions Schedule (MUPS) selected from the group consisting of onset and course, number, volume, tone, and location; (c) auditory hallucination; (d) tactile hallucination; (e) visual hallucination; (f) olfactory hallucination; (g) gustatory hallucination; (h) delusions; (i) proprioceptive hallucination; (j) equilibrioceptive hallucination; (k) nociceptive hallucination; (l) thermoceptive hallucination; (m) chronoceptive hallucination; (n) non-auditory command hallucination; (o) psychosis; (p) peduncular hallucinosis; (p) delirium; (r) dementia; (s) neurodegenerative disease; (t) neurodegeneration; (u) epilepsy; (v) seizures; (w) migraines; (x) cognitive impairment; (y) constipation; (z) depression; (aa) sleep problem, sleep disorder, or sleep disturbance; and/or (bb) gastrointestinal disorders. The symptoms can be measured using a clinically recognized scale or tool, as detailed herein.

Example 4 provides a detailed protocol for determining a “fixed dose” based on improvement of one symptom associated with Parkinson's disease (PD), e.g., constipation. This example further details how this “fixed dose” successfully treated not only constipation, but also other non-dopamine related symptoms of PD, which therefore are applicable to treatment of hallucinations.

As dopaminergic activity distinguishes PD from other neurodegenerative disorders and these data relate to symptoms that do not relate to this distinguishing feature, this dosing regime is believed to be extrapolatable both to other symptoms and other disorders including hallucinations.

Not to be bound by theory, it is believed that establishing a patient-specific “fixed dose” based on hitting a threshold improvement in any of the symptoms listed below and administering this therapeutically effective fixed dose will successfully treat the initial symptom and one or more of the other symptoms. Further, to the extent that these symptoms are tied to an underlying disorder, administration of the therapeutically effective fixed dose is also believed to offer a means of treating, preventing, and/or delaying onset of the underlying hallucination associated disorder.

I. Hallucination

There are currently a variety of art-accepted methods for diagnosing and/or measuring hallucinations quantitatively and qualitatively. Thus, in some embodiments, (a) the positive impact and/or progression of hallucinations and/or related symptoms is measured quantitatively or qualitatively by one or more techniques selected from the group consisting of Chicago Hallucination Assessment Tool (CHAT), The Psychotic Symptom Rating Scales (PSYRATS), Auditory Hallucinations Rating Scale (AHRS), Hamilton Program for Schizophrenia Voices Questionnaire (HPSVQ), Characteristics of Auditory Hallucinations Questionnaire (CAHQ), Mental Health Research Institute Unusual Perception Schedule (MUPS), positive and negative syndrome scale (PANSS), scale for the assessment of positive symptoms (SAPS), Launay-Slade hallucinations scale (LSHS), the Cardiff anomalous perceptions scale (CAPS), and structured interview for assessing perceptual anomalies (SIAPA); and/or (b) the progression or onset of hallucinations and/or related symptoms is slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique. The methods of the invention may also result in the subject being auditory hallucination-free.

Progression of neurodegeneration-associated with hallucinations can be measured using well known techniques. In some embodiments, (a) the positive impact and/or progression of neurodegeneration is measured quantitatively or qualitatively by one or more techniques selected from the group consisting of electroencephalogram (EEG), neuroimaging, functional MRI, structural MRI, diffusion tensor imaging (DTI), [18F]fluorodeoxyglucose (FDG) PET, agents that label amyloid, [18F]F-dopa PET, radiotracer imaging, volumetric analysis of regional tissue loss, specific imaging markers of abnormal protein deposition, multimodal imaging, and biomarker analysis; and/or (b) the progression or onset of neurodegeneration is slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique.

The period of time over which the progression or onset of neurodegeneration is measured can be for example, one or more months or one or more years, e.g., about 6 months, about 1 year, about 18 months, about 2 years, about 36 months, about 3, about 4, about 5, about 6, about 7, about 8, about 9, about 10, about 11, about 12, about 13, about 14, about 15, about 16, about 17, about 18, about 19, or about 20 years, or any amount of months or years in between the values of about 6 months to about 20 years or more.

Example 4 describes several tools used to measure and evaluate the effect of aminosterol treatment on hallucinations, including for example:

(1) The University of Miami Parkinson's Disease Hallucinations Questionnaire (UM-PDHQ);

(2) Unified Parkinson's Disease Scale (UPSRS), section 1.2 (Hallucinations and Psychosis); and

(3) direct questioning.

As described in Example 4, the PDHQ score improved from 1.3 at baseline to 0.9 during wash-out. Hallucinations were reported by 5 patients at baseline and delusions in 1 patient. Both hallucinations and delusions improved or disappeared in 5 of 6 patients during treatment and did not return for 4 weeks following discontinuation of aminosterol treatment in 1 patient and 2 weeks in another. In one patient the hallucinations disappeared at 100 mg, despite not having reached the colonic prokinetic dose at 175 mg. Further, unlike stool-related indices, the improvement in many CNS symptoms persisted during wash-out.

2 Hallucination Symptoms

Symptoms of hallucinations that can be used as markers to determine dosage of an aminosterol or a salt or derivative thereof are described herein, with several symptoms detailed more extensively below.

i. Constipation

While often dismissed as strictly a gastrointestinal symptom, constipation is believed to be an early indicator of neurodegenerative disease to the extent that ENS degeneration can be indicative of later CNS degeneration. Indeed, not to be bound by theory, constipation is observed in patients with hallucinations. Accordingly, method embodiments disclosed herein relate to the treatment of constipation which is a symptom associated with hallucination and neurodegeneration or the treatment and/or prevention of the underlying hallucination precipitating disorder associated with constipation.

Constipation is defined as a lower than normal frequency of bowel movements in a fixed duration of time (e.g. less than 3 bowel movements per week). Constipation not only constitutes a major economic burden, but it also significantly affects the quality of life of the individual, contributing to social isolation and depression. Furthermore, the severity of the symptoms correlates negatively with patient reported quality of life.

Example 4 describes several tools used to measure and evaluate the effect of aminosterol treatment on constipation, including for example:

(1) Rome-IV Criteria for Constipation (7 criteria, with constipation diagnosis requiring two or more of the following: (i) straining during at least 25% of defecations, (ii) lumpy or hard stools in at least 25% of defecations, (iii) sensation of incomplete evacuation for at least 25% of defecations, (iv) sensation of anorectal obstruction/blockage for at least 25% of defecations; (v) manual maneuvers to facilitate at least 25% of defecations; (vi) fewer than 3 defecations per week; and (vii) loose stools are rarely present without the use of laxatives;

(2) Constipation—Ease of Evacuation Scale (from 1-7, with 7=incontinent, 4=normal, and 1=manual disimpaction);

(3) Bristol Stool Chart, which is a patient-friendly means of categorizing stool characteristics (assessment of stool consistency is a validated surrogate of intestinal motility) and stool diary;

(4) Unified Parkinson's Disease Scale (UPSRS), section 1.11 (Constipation Problems);

(5) Patient Assessment of Constipation Symptoms (PAC-SYM); and

(5) Patient Assessment of Constipation Quality of Life (PAC-QOL).

Examples of characteristics of constipation that can be positively affected by the method of the invention include, but are not limited to, frequency of constipation, duration of constipation symptoms, bowel movement frequency, stool consistency, abdominal pain, abdominal bloating, incomplete evacuation, unsuccessful attempts at evacuation, pain with evacuation, and straining with evacuation. Potentially all of these characteristics can be positively impacted by the methods of the invention. Further, assessments of these characteristics are known in the art, e.g. spontaneous bowel movements (SBMs)/week, stool consistency (Bristol Stool Form Scale) (Heaton et al. 1992), ease of passage (Ease of Evacuation Scale) (Andresen et al. 2007), rescue medication use and symptoms and quality of life related to bowel function (PAC-SYM (Frank et al. 1999) and PAC-QOL (Marquis et al. 2005)).

The methods of using a composition comprising a therapeutically effective fixed dose of an amino sterol, or a salt or derivative thereof, according to the invention to treat and/or prevent constipation associated with hallucinations preferably results in an increase in the number of spontaneous bowel movements per week and/or an improvement in other stool conditions. The increase can be, for example, an increase of between 1 to 3 spontaneous bowel movements in a week, or, optionally, full restoration of regular bowel function.

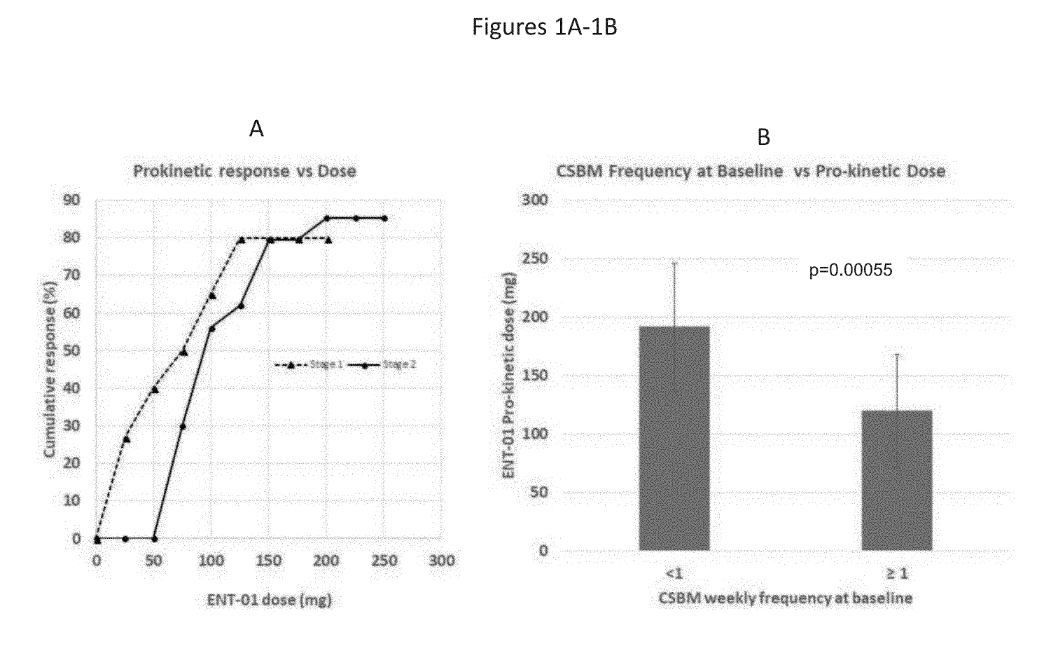

Data detailed in Example 4 shows that 80% of subjects responded to aminosterol treatment with improved bowel function (see FIG. 1A), with the cumulative response rate increasing in a dose-dependent fashion from 25% at 25 mg to a maximum of 80% at 200 mg (Stage 1, FIG. 1A). In Stage 2 of the study, the response rate increased in a dose-dependent fashion from 26% at 75 mg to 85.3% at 250 mg (FIG. 1A). The dose required for a bowel response was patient-specific and varied from 75 mg to 250 mg. The median efficacious dose was 100 mg.

The average CSBM/week increased from 1.2 at baseline to 3.8 at fixed dose (216% improvement) and SBM increased from 2.6 at baseline to 4.5 at fixed dose (73% improvement). Use of rescue medication decreased from 1.8/week at baseline to 0.3 at fixed dose (83% decrease). Consistency based on the Bristol stool scale also improved, increasing from mean 2.7 to 4.1 (52% improvement) and ease of passage increased from 3.2 to 3.7 (16% improvement). Subjective indices of wellbeing (PAC-QOL) and constipation symptoms (PAC-SYM) also improved during treatment.

The dose that proved efficacious in inducing a bowel response was strongly related to constipation severity at baseline (FIG. 1B); patients with baseline constipation of <1 CSBM/week required higher doses for a response (mean 192 mg) than patients with ≥1 CSBM/week (mean 120 mg).

In one embodiment of the invention, treatment of a hallucination subject having constipation with an aminosterol or a salt or derivative thereof in a method described herein results in an improvement of one or more characteristics of constipation associated with hallucination. The improvement can be, for example, about 5, about 10, about 15, about 20, about 25, about 30, about 35, about 40, about 45, about 50, about 55, about 60, about 65, about 70, about 75, about 80, about 85, about 90, about 95, about 100, about 110, about 120, about 130, about 140, about 150, about 160, about 170, about 180, about 190, about 200, about 210, about 220, about 230, about 240, about 250, about 260, about 270, about 280, about 290, about 300, about 325, about 350, about 375 or about 400%. Examples of constipation characteristics that can be improved by the methods of the invention include, but are not limited to, frequency of constipation, duration of constipation symptoms, bowel movement frequency, stool consistency, abdominal pain, abdominal bloating, incomplete evacuation, unsuccessful attempts at evacuation, pain with evacuation, and straining with evacuation. Measurement of a constipation characteristic can be done using any clinically recognized scale or tool.

One surprising discovery that resulted from the experiments described herein related to amino sterol dosing. It was surprisingly discovered that the dose of aminosterol required to obtain a positive impact on a hallucination symptom being evaluated, referred to herein as a “fixed escalated aminosterol dose,” is patient specific. Moreover, it was discovered that the fixed escalated aminosterol dose is not dependent upon age, size, or weight but rather is individually calibrated. Further, it was discovered that the severity of constipation correlates with a higher required “fixed escalated amino sterol dose.” It is theorized that the amino sterol dose required to obtain a positive effect in a subject for the symptom being evaluated correlates with the extent of neuronal damage. Thus, it is theorized that greater neuronal damage correlates with a higher required aminosterol dose to obtain a positive effect in a subject for the symptom being evaluated. The observation that the aminosterol dose required to achieve a desired response increases with constipation severity supports the hypothesis that the greater the burden of αS impeding neuronal function, the higher the dose of amino sterol required to restore normal bowel function. Moreover, the data described in Example 4 confirms the hypothesis that gastrointestinal dysmotility in PD results from the progressive accumulation of αS in the ENS, and that amino sterol treatment can restore neuronal function by displacing αS and stimulating enteric neurons. These results demonstrate that the ENS in PD is not irreversibly damaged and can be restored to normal function.

In calibrating the fixed amino sterol dose for a specific hallucination patient, the starting dose is varied based upon the severity of the constipation (when constipation is used as the hallucination symptom to be evaluated). Thus, for subjects with severe constipation, e.g., subjects with 1 or less CSBM or SMB per week, oral aminosterol dosing is started at about 100 to about 175 mg or more (or any amount in-between these values as described herein). For subjects with less severe constipation, e.g., more than 1 CSBM or SBM per week, oral aminosterol dosing is started at about 25 to about 75 mg (or any amount in-between these values as described herein). Dosing for both patients is then escalated by defined amounts over a defined period of time until the fixed escalated dose for the patient is identified. Amino sterol doses can also be de-escalated (reduced) if any given aminosterol dose induces a persistent undesirable side effect, such as diarrhea, vomiting, or nausea.

For example, for patients with severe constipation, a starting oral aminosterol dosage can be from 75 mg up to about 300 mg, or any amount in-between these two values. In other embodiments, the starting oral aminosterol dosage for severely constipated patients can be, for example, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, or about 300 mg. A “fixed escalated” oral aminosterol dose for a severely constipated patient is likely to range from about 75 mg up to about 500 mg. As described in Example 4, a positive effect was defined as a dose that resulted in a CSBM within 24 hours of dosing on at least 2 of 3 days at a given dose.

For patients with less severe constipation, oral aminosterol dosing is started at about 10 to about 75 mg, or any amount in-between these two values as described herein. For example, starting oral aminosterol dosage for patients with moderate to mild constipation can be about 1, about 5, about 10, about 15, about 20, about 25, about 30, about 35, about 40, about 45, about 50, about 55, about 60, about 65, about 70, up to less than or equal to about 75 mg. A fixed escalated oral aminosterol dose for a mild or moderately constipated patient is likely to range from about 5 mg up to about 350 mg, or any amount in-between these two values as described herein.

ii. Depression

Another symptom associated with hallucinations is depression. Clinical depression is characterized by a sad, blue mood that goes above and beyond normal sadness or grief. Major depression is an episode of sadness or apathy along with other symptoms that lasts at least two consecutive weeks and is severe enough to interrupt daily activities. Depressive events feature not only negative thoughts, moods, and behaviors but also specific changes in bodily functions (like, eating, sleeping, energy and sexual activity, as well as potentially developing aches or pains). One in 10 people will have a depression in their lifetime. Doctors clinically diagnose depression; there is no laboratory test or X-ray for depression.

Increasingly sophisticated forms of brain imaging, such as positron emission tomography (PET), single-photon emission computed tomography (SPECT), and functional magnetic resonance imaging (fMRI), permit a much closer look at the working brain than was possible in the past. An fMRI scan, for example, can track changes that take place when a region of the brain responds during various tasks. A PET or SPECT scan can map the brain by measuring the distribution and density of neurotransmitter receptors in certain areas. Use of this technology has led to a better understanding of which brain regions regulate mood and how other functions, such as memory, may be affected by depression. Areas that play a significant role in depression are the amygdala, the thalamus, and the hippocampus.

Research shows that the hippocampus is smaller in some depressed people. For example, in one fMRI study published in The Journal of Neuroscience, investigators studied 24 women who had a history of depression. On average, the hippocampus was 9% to 13% smaller in depressed women as compared with those who were not depressed. The more bouts of depression a woman had, the smaller the hippocampus. Stress, which plays a role in depression, may be a key factor, since experts believe stress can suppress the production of new neurons (nerve cells) in the hippocampus.

Researchers are exploring possible links between sluggish production of new neurons in the hippocampus and low moods. An interesting fact about antidepressants supports this theory. These medications immediately boost the concentration of chemical messengers in the brain (neurotransmitters). Yet people typically don't begin to feel better for several weeks or longer. Experts have long wondered why, if depression were primarily the result of low levels of neurotransmitters, people don't feel better as soon as levels of neurotransmitters increase. The answer may be that mood only improves as nerves grow and form new connections, a process that takes weeks. In fact, animal studies have shown that antidepressants do spur the growth and enhanced branching of nerve cells in the hippocampus. So, the theory holds, the real value of these medications may be in generating new neurons (a process called neurogenesis), strengthening nerve cell connections, and improving the exchange of information between nerve circuits.

Thus, in one embodiment of the invention, encompassed are methods of treating, preventing and/or slowing the onset or progression of depression in hallucination subjects comprising administering a therapeutically effective fixed dose of an aminosterol composition according to the invention. While not wishing to be bound by theory, it is theorized that the aminosterol compositions of the invention trigger neurogenesis, which functions to combat depression.

In some embodiments, the methods of the invention produce an improvement in a hallucination subject's clinical depression. An improvement in a hallucination subject's depression can be measured using any clinically-recognized measurement. For example, improvement can be measured using a depression rating scale. In one embodiment of the invention, following treatment a subject experiences an about 5, about 10, about 15, about 20, about 25, about 30, about 35, about 40, about 45, about 50, about 55, about 60, about 65, about 70, about 75, about 80, about 85, about 90, about 95 or an about 100% improvement. The improvement can be measured using any clinically recognized tool or assessment.

As detailed in Example 4, depression and/or mood and the improvement following aminosterol treatment were assessed using several tools:

(1) Beck Depression Inventory (BDI-II);

(2) Unified Parkinson's Disease Rating Scale (UPDRS), sections 1.3 (depressed mood), 1.4 (anxious mood), 1.5 (apathy), and 1.13 (fatigue); and

(3) Parkinson's Disease Fatigue Scale (PFS-16).

Assessments were made at baseline and at the end of the fixed dose and washout periods. An analysis was done with respect to depression and mood scores. Total UPDRS score was 64.4 at baseline, 60.6 at the end of the fixed dose period and 55.7 at the end of the wash-out period, demonstrating a 13.5% improvement, and Part 1 of the UPDRS (which includes mood and depression scores) went from a mean score of 11.6 at baseline, to a mean of 10.6 during the fixed amino sterol dose period, with a mean score of 9.5 during the washout period, demonstrating an improvement of 18%. In addition, BDI-II scores decreased from 10.9 at baseline to 9.9 during treatment and 8.7 at wash-out, showing an improvement in depression scoring of 20%. Unlike stool-related indices, the improvement in many CNS symptoms persisted during wash-out.

C. Aminosterols

As described herein, the present invention relates to methods of treating, preventing, and/or slowing the onset or progression of hallucinations and/or a hallucination-related symptom in a subject in need thereof. The methods comprise administering a therapeutically effective amount of one or more aminosterols or a pharmaceutically equivalent derivative or salt thereof to a subject in need. A “subject in need thereof” is a human suffering from or at risk of suffering from hallucinations. By administering an aminosterol, hallucinations can be treated and/or prevented.

U.S. Pat. No. 6,962,909, entitled “Treatment of neovascularization disorders with squalamine,” discloses various aminosterols, and this disclosure is specifically incorporated herein by reference with respect to its teaching of aminosterol compounds. Any aminosterol known in the art, including those described in U.S. Pat. No. 6,962,909, can be used in the disclosed methods.

An aminosterol such as squalamine (ENT-01 in the examples) inhibits the formation of αS aggregates in vitro and in vivo, reverses motor dysfunction in the C. elegans αS model, and restores gastrointestinal motility in mouse models of PD.

Squalamine (ENT-01) has limited bioavailability in rats and dogs. Based on measurement of portal blood concentrations following oral dosing of radioactive ENT-01 to rat's absorption of ENT-01 from the intestine is low. As a consequence, the principal focus of safety is on local effects on the GIT. However, squalamine (ENT-01) appears to be well tolerated in both rats and dogs.

For instance, useful amino sterol compounds comprise a bile acid nucleus and a polyamine, attached at any position on the bile acid, such that the molecule exhibits a net positive charge contributed by the polyamine.

Thus, in some embodiments, the disclosed methods comprise administering a therapeutically effective amount of one or more aminosterols having the chemical structure of Formula I:

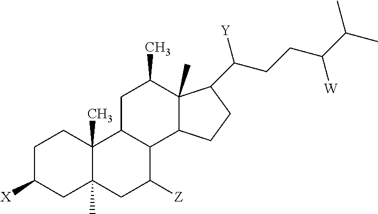

wherein,
W is 24S —OSO₃ or 24R—OSO₃;
X is 3β-H₂N—(CH₂)₄—NH—(CH₂)₃—NH— or 3α-H₂N—(CH₂)₄—NH—(CH₂)₃—NH—;

### Y is 20R- CH₃; and

### Z is 7α or 7β —OH.

In another embodiment of the invention, the amino sterol is one of the naturally occurring aminosterols (1-8) isolated from Squalus acanthias:

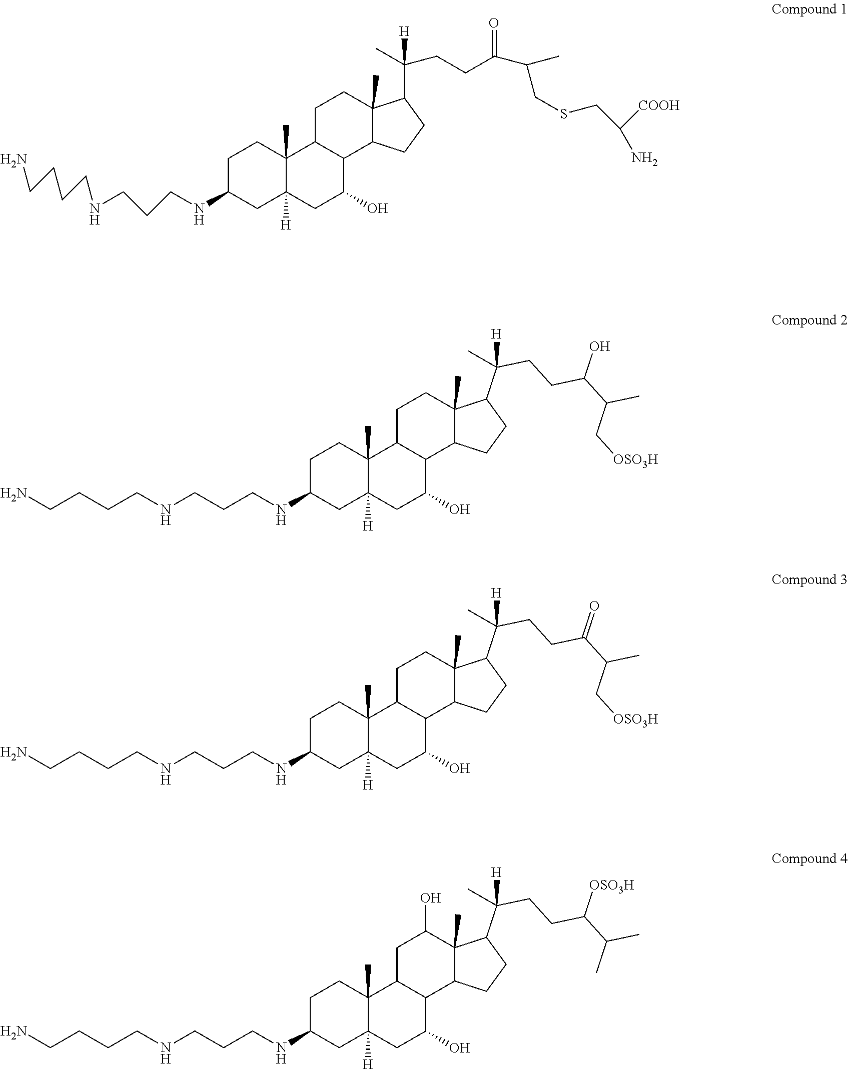

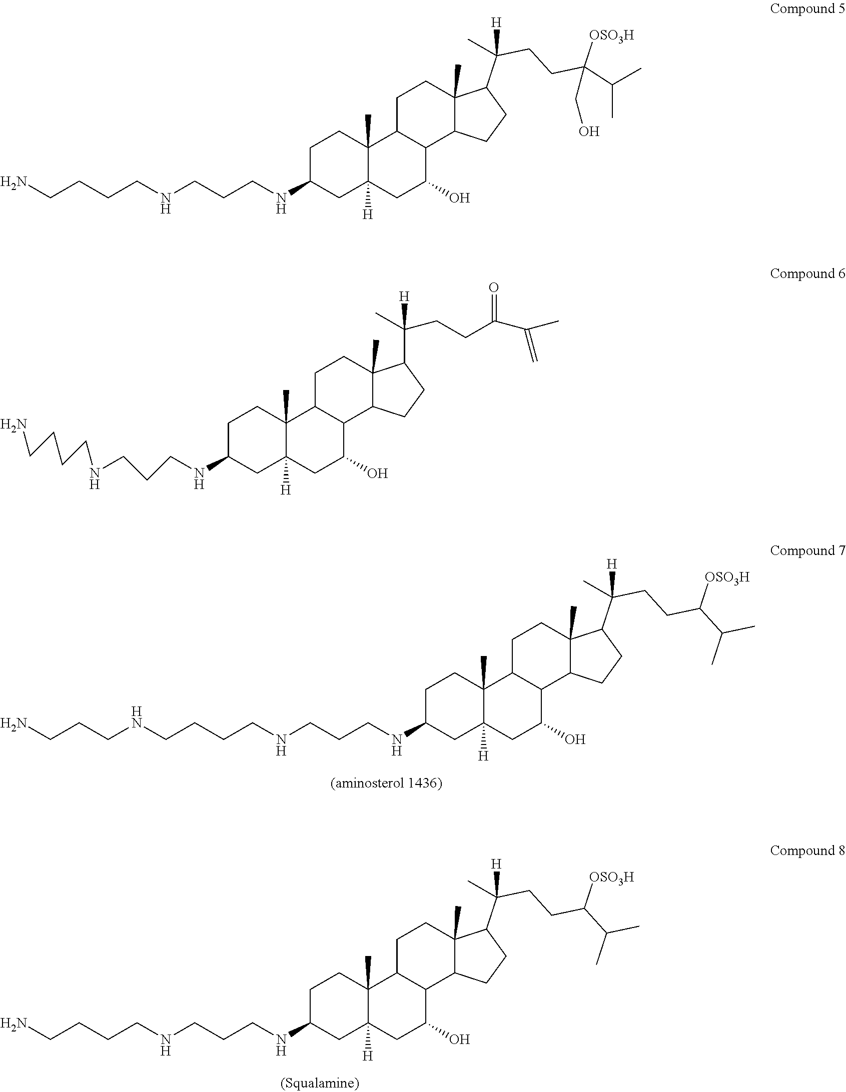

Variants, salts, or derivatives of known aminosterols, such as squalamine, Aminosterol 1436, or an aminosterol isolated from Squalus acanthias, may be used in the disclosed compositions and methods. In one aspect of the invention, the aminosterol is Aminosterol 1436 or a salt or derivative thereof. In another embodiment the aminosterol is squalamine or a salt or derivative thereof.

Any pharmaceutically acceptable salt of an amino sterol can be used in the compositions and methods of the invention. For example, a phosphate salt or buffer, free base, succinate, phosphate, mesylate or other salt form associated with low mucosal irritation can be utilized in the methods and compositions of the invention. In some embodiments, the methods of the invention can employ a formulation of Aminosterol 1436 or squalamine as an insoluble salt of phosphate, polyphosphate, or an organic phosphate ester.

In yet another embodiment, the aminosterol comprises a sterol nucleus and a polyamine, attached at any position on the sterol, such that the molecule exhibits a net charge of at least +1, the charge being contributed by the polyamine. In yet another embodiment, the aminosterol comprises a bile acid nucleus and a polyamine, attached at any position on the bile acid, such that the molecule exhibits a net positive charge being contributed by the polyamine.

In some embodiments, the compositions used in the methods of the invention comprise: (a) at least one pharmaceutical grade aminosterol; and optionally (b) at least one phosphate selected from the group consisting of an inorganic phosphate, an inorganic pyrophosphate, and an organic phosphate. In some embodiments, the aminosterol is formulated as a weakly water soluble salt of the phosphate. In some embodiments, the phosphate is an inorganic polyphosphate, and the number of phosphates can range from about 3 (tripolyphosphate) to about 400, or any number in-between these two values. In other embodiments, the phosphate is an organic phosphate which comprises glycerol 2 phosphates.

In some embodiments, the amino sterol is selected from the group consisting of: (a) squalamine or a pharmaceutically acceptable salt or derivative thereof; (b) a squalamine isomer; (c) a squalamine phosphate salt; (d) Aminosterol 1436 or a pharmaceutically acceptable salt or derivative thereof; (e) an isomer of aminosterol 1436; (f) an aminosterol 1436 phosphate salt, (g) a synthetic aminosterol; (h) an aminosterol comprising a sterol or bile acid nucleus and a polyamine, attached at any position on the sterol or bile acid, such that the molecule exhibits a net charge of at least +1, the charge being contributed by the polyamine; (i) an amino sterol which is a derivative of squalamine or another naturally occurring amino sterol modified through medical chemistry to improve biodistribution, ease of administration, metabolic stability, or any combination thereof; (f) an amino sterol modified to include one or more of the following: (i) substitutions of the sulfate by a sulfonate, phosphate, carboxylate, or other anionic moiety chosen to circumvent metabolic removal of the sulfate moiety and oxidation of the cholesterol side chain; (ii) replacement of a hydroxyl group by a non-metabolizable polar substituent, such as a fluorine atom, to prevent its metabolic oxidation or conjugation; and (iii) substitution of various ring hydrogen atoms to prevent oxidative or reductive metabolism of the steroid ring system; (g) an aminosterol that can inhibit the formation of actin stress fibers in endothelial cells stimulated by a ligand known to induce stress fiber formation, having the chemical structure of Formula I (above); or (j) any combination thereof.

In some embodiments, the compositions used in the methods of the invention comprise: (a) at least one pharmaceutical grade aminosterol; and optionally (b) at least one phosphate selected from the group consisting of an inorganic phosphate, an inorganic pyrophosphate, and an organic phosphate. In some embodiments, the aminosterol is formulated as a weakly water soluble salt of the phosphate. In some embodiments, the phosphate is an inorganic polyphosphate, and the number of phosphates can range from about 3 (tripolyphosphate) to about 400, or any number in-between these two values. In other embodiments, the phosphate is an organic phosphate which comprises glycerol 2 phosphates.

In some embodiments, the amino sterol can be composed of a sterol or bile acid nucleus to which a polyamine is chemically linked, displaying a net positive charge of at least +1. The methods can be embodied in a formulation comprising a phosphate suspension or as a tablet for oral administration. As an oral formulation, squalamine phosphate (or another aminosterol phosphate) slowly dissolves in the gastrointestinal tract, and does not subject the lining of the intestine to high local concentrations that would otherwise irritate or damage the organ.

In certain embodiments of the invention, the methods comprise administering squalamine or a derivative thereof at an effective daily dosing amount of about 0.1 to about 20 mg/kg body weight. In certain embodiments, the effective dose can be established by defining the initial dose required to induce the Aminosterol-Induced GI Response, i.e., the initial dose required to stimulate nausea and secretory diarrhea. In other embodiments, the effective daily dosing amount is about 0.1, about 0.5, about 1, about 2, about 3, about 4, about 5, about 6, about 7, about 8, about 9, about 10, about 11, about 12, about 13, about 14, about 15, about 16, about 17, about 18, about 19, or about 20 mg/kg body weight.

Mechanism of action. It has been reported that's squalamine exerts its effects at the cellular level by displacing proteins bound electrostatically to negatively charged membranes, causing pleiotropic changes in the functional state of the cell. See Alexander et al. (2011); Yeung et al. (2008); Sumioka et al. (2009); and Zasloff et al. (2011). With respect to the disclosed methods, it is believed that squalamine and other aminosterols, such as Aminosterol 1436, are not necessarily absorbed in the gastrointestinal (GI) tract but may nevertheless produce an aminosterol-induced central nervous system (CNS) response. The presence of the aminosterol may induce various cellular-level responses, including effects on water and salt reabsorption. The aminosterol may also induce electrical activation of specific neurons, ultimately, by the electrostatic mechanism proposed.

Squalamine is known to gain access to nerve cells, neutralize the negative electrostatic surface potential of these cells, and alter electrical channel activity (Sumioka et al., (2009)). Without being bound by a particular theory, it is assumed that squalamine can access and influence the behavior of the neurons of the enteric nervous system in a fashion similar to what has been observed in cortical granular neurons (Sumioka et al., (2009)). In addition, squalamine is known to inhibit the sodium hydrogen exchanger involved in water and salt reabsorption in the human small intestine by the same mechanism (Alexander et al. (2011)).

Without intending to be bound by theory, one proposed mechanism by which an aminosterol provokes the aminosterol-induced response involves the direct stimulation of nerves within the enteric nervous system, and stimulation of currents flowing towards the brain through afferent nerves of the vagus, which is predominantly parasympathetic and cholinergic. However, stimulation of other afferent neurons from gut to brain, including sympathetic nerves and sensory nerves, may also be involved in producing the desired affects. Stimulation of afferents of the vagus, which distribute to centers and tracts within the brain would be expected to stimulate release of a suite of neuropeptides within the brain itself. The continued imposition of the ileal brake for several days following amino sterol dosing, speaks to the length of time the aminosterol-provoked gut/CNS interaction must be operative following a single dose of amino sterol.

In addition, the entry of amino sterols into the nerves of a subject in need thereof could provide direct benefit in reducing hallucinations associated with degenerative conditions where accumulation of certain proteins is believed to be causally involved. For example, accumulation of misfolded oligomers and larger aggregates of α-synuclein defines multiple neurodegenerative diseases called synucleinopathies, including Parkinson's Disease. (Burre et al. 2018). Consistent with a role for α-synuclein accumulation in causing hallucination, α-synuclein deposits in the stratum griseum intermedium, an important structure in directing attention toward visual targets, were observed in dementia with Lewy bodies patients that exhibits visual hallucinations, but not in Alzheimer's patients without visual hallucinations. (Erskine et al., 2017). Alpha synuclein is a protein with a cationic N-terminus and can interact electrostatically with the internal membranes of the nerve cell in which it is expressed. Since amino sterols (e.g., squalamine) can both enter nerve cells and neutralize the negative surface potential of these membrane surfaces, squalamine and related amino sterols have the capacity to displace alpha synuclein from membrane sites within nerves, and as a consequence, interrupt the pathophysiology of the disease. Accordingly, without being bound by theory, squalamine and Aminosterol 1436 may ameliorate hallucination by displacing alpha-synuclein. In addition, squalamine and Aminosterol 1436 may increase nerve cell firing rates and duration to therefore ameliorate hallucinations.

### D. Routes of Administration

It is appreciated that the “fixed dose” disclosed herein can be administered via any suitable route of administration, including but not limited to oral or intranasal delivery, injection (IP, IV, or IM), or a combination thereof.

Further, co-administration of the “fixed dose” with injectable (e.g., 1P, IV, IM) aminosterol formulations is also contemplated herein. For injectable dosage forms, the dosage form can comprise an aminosterol at a dosage of, for example, about 0.1 to about 20 mg/kg body weight. In other embodiments, the effective daily dosing amount is about 0.1, about 0.5, about 1, about 2, about 3, about 4, about 5, about 6, about 7, about 8, about 9, about 10, about 11, about 12, about 13, about 14, about 15, about 16, about 17, about 18, about 19, or about 20 mg/kg body weight.

The invention also encompasses methods of treatment using a combination of an aminosterol composition administered via one route, e.g., oral, with a second amino sterol composition, comprising the same or a different amino sterol, administered via a different route, e.g., intranasal. For example, in a method of the invention, squalamine can be administered orally and aminosterol 1436 can be administered IN.

In one embodiment of the disclosed methods, following oral administration there is essentially no detectable levels of the administered aminosterol in the bloodstream of the subject. In another embodiment, following oral administration there is preferably less than about 10 ng/ml of the administered aminosterol in the bloodstream of the subject, measured between about 1 to about 12 hours following oral administration. In other embodiments, following oral administration there is less than about 9, less than about 8, less than about 7, less than about 6, less than about 5, less than about 4, less than about 3, less than about 2, or less than about 1 ng/ml in the bloodstream of the subject measured from about 1 to about 12 hours following oral administration.

In one embodiment, administering comprises nasal administration. Nasal administration may be accomplished via insufflation of solids, liquids or powders, inhalation of a gas, or via inhalation of a mist comprising the at least one aminosterol in a suitable carrier and optionally excipients. Suitable carriers and excipients are known to the skilled artisan and include buffers such as sodium phosphate, sodium citrate, and citric acid; solubilizers such as glycols, small quantities of alcohol, transcutol (diethylene glycol monoethyl ether), medium chain glycerides, labrasol (saturated polyglycolyzed C₈-C₁₀ glyceride), surfactants and cyclodextrins; preservatives such as parabens, phenyl ethyl alcohol, benzalkonium chloride, EDTA (ethylene diaminetetraaceticacid), and benzoyl alcohol; antioxidants such as sodium bisulfite, butylated hydroxytoluene, sodium metabisulfite and tocopherol; humectants such as glycerin, sorbitol and mannitol; surfactants such as polysorbet; bioadhesive polymers such as mucoadhesives; and penetration enhancers such as dimethyl sulfoxide (DMSO).

Nasal administration via inhalation of a mist may employ the use of metered-dose spray pumps. Typical volumes of aminosterol-comprising mist, delivered via a single pump of a metered-dose spray pump may be about 20-100 μl, 100-150 μl, or 150-200 μl. Such pumps offer high reproducibility of the emitted dose and plume geometry. The particle size and plume geometry can vary within certain limits and depend on the properties of the pump, the formulation, the orifice of the actuator, and the force applied.

### E. Composition Components

In some embodiments, a pharmaceutical composition disclosed herein comprises one or more pharmaceutically acceptable carriers, such as an aqueous carrier, buffer, and/or diluent.

In some embodiments, a pharmaceutical composition disclosed herein further comprises a simple polyol compound, such as glycerin. Other examples of polyol compounds include sugar alcohols. In some embodiments, a pharmaceutical composition disclosed herein comprises an aqueous carrier and glycerin at about a 2:1 ratio.

The formulations may conveniently be presented in unit dosage form and may be prepared by any of the methods well known in the art of pharmacy. An exemplary oral dosage form is a tablet or capsule. An exemplary intranasal dosage form is a liquid or powder nasal spray. A nasal spray is designed to deliver drug to the upper nasal cavity, and can be a liquid or powder formulation, and in a dosage form such as an aerosol, liquid spray, or powder.

The amino sterol may be combined or coordinately administered with a suitable carrier or vehicle depending on the route of administration. As used herein, the term “carrier” means a pharmaceutically acceptable solid or liquid filler, diluent or encapsulating material. A water-containing liquid carrier can comprise pharmaceutically acceptable additives such as acidifying agents, alkalizing agents, antimicrobial preservatives, antioxidants, buffering agents, chelating agents, complexing agents, solubilizing agents, humectants, solvents, suspending and/or viscosity-increasing agents, tonicity agents, wetting agents or other biocompatible materials. A tabulation of ingredients listed by the above categories can be found in the U.S. Pharmacopeia National Formulary, 1857-1859, and (1990). Some examples of the materials which can serve as pharmaceutically acceptable carriers are sugars, such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols, such as propylene glycol; polyols such as glycerin, sorbitol, mannitol and polyethylene glycol; esters such as ethyl oleate and ethyl laurate; agar; buffering agents such as magnesium hydroxide and aluminum hydroxide; alginic acid; pyrogen free water; isotonic saline; Ringer's solution, ethyl alcohol and phosphate buffer solutions, as well as other nontoxic compatible substances used in pharmaceutical formulations. Wetting agents, emulsifiers and lubricants such as sodium lauryl sulfate and magnesium stearate, as well as coloring agents, release agents, coating agents, sweetening, flavoring and perfuming agents, preservatives and antioxidants can also be present in the compositions, according to the desires of the formulator. Examples of pharmaceutically acceptable antioxidants include water soluble antioxidants such as ascorbic acid, cysteine hydrochloride, sodium bisulfite, sodium metabisulfite, sodium sulfite and the like; oil-soluble antioxidants such as ascorbyl palmitate, butylated hydroxyanisole (BHA), butylated hydroxytoluene (BHT), lecithin, propyl gallate, alpha-tocopherol and the like; and metal-chelating agents such as citric acid, ethylenediamine tetraacetic acid (EDTA), sorbitol, tartaric acid, phosphoric acid and the like.

Pharmaceutical compositions according to the invention may also comprise one or more binding agents, filling agents, lubricating agents, suspending agents, sweeteners, flavoring agents, preservatives, buffers, wetting agents, disintegrants, effervescent agents, and other excipients. Such excipients are known in the art. Examples of filling agents include lactose monohydrate, lactose anhydrous, and various starches; examples of binding agents include various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC™). Suitable lubricants, including agents that act on the flowability of the powder to be compressed, may include colloidal silicon dioxide, such as Aerosil® 200, talc, stearic acid, magnesium stearate, calcium stearate, and silica gel. Examples of sweeteners may include any natural or artificial sweetener, such as sucrose, xylitol, sodium saccharin, cyclamate, aspartame, and acesulfame. Examples of flavoring agents are Magnasweet® (trademark of MAFCO), bubble gum flavor, and fruit flavors, and the like. Examples of preservatives include potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride.

Suitable diluents include pharmaceutically acceptable inert fillers, such as microcrystalline cellulose, lactose, dibasic calcium phosphate, saccharides, and/or mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose. In some embodiments, a pharmaceutical composition disclosed herein further comprises a simple polyol compound, such as glycerin. Other examples of polyol compounds include sugar alcohols. In some embodiments, a pharmaceutical composition disclosed herein comprises an aqueous carrier and glycerin at about a 2:1 ratio.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof. Examples of effervescent agents include effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

Any pharmaceutical used for therapeutic administration can be sterile. Sterility is readily accomplished by for example filtration through sterile filtration membranes (e.g., 0.2 micron membranes). Any pharmaceutically acceptable sterility method can be used in the compositions of the invention.

The pharmaceutical composition comprising an aminosterol derivatives or salts thereof will be formulated and dosed in a fashion consistent with good medical practice, taking into account the clinical condition of the individual patient, the method of administration, the scheduling of administration, and other factors known to practitioners.

F. Dosage Forms

Various formulations may be used for administration of the disclosed aminosterols. The formulations may conveniently be presented in unit dosage form and may be prepared by any of the methods well known in the art of pharmacy. Any pharmaceutically acceptable dosage form may be employed in the methods of the invention. For example, the composition can be formulated into a dosage form selected from the group consisting of liquid dispersions, gels, aerosols, lyophilized formulations, tablets, or capsules. In some embodiments, the amino sterol may be incorporated into a dosage form selected from the group consisting of controlled release formulations, fast melt formulations, delayed release formulations, extended release formulations, pulsatile release formulations, and mixed immediate release and controlled release formulations. In some embodiments, the dosage form may comprise a combination of the forgoing formulation options (e.g., a controlled release tablet).

In one embodiment of the invention, the oral dosage form is a liquid, capsule, or tablet designed to disintegrate in either the stomach, upper small intestine, or more distal portions of the intestine with a dissolution rate appropriate to achieve the intended therapeutic benefit.

An exemplary dosage form is an orally administered dosage form, such as a tablet or capsule. These dosage forms can be formulated by any method known in the art. Such methods include the step of bringing into association the aminosterol with the carrier that constitutes one or more accessory ingredients. In general, the formulations are prepared by uniformly and intimately bringing into association the active ingredient with liquid carriers or finely divided solid carriers or both, and then, if necessary, shaping the product. Another example of an exemplary dosage form is a nasal spray, comprising a dry powder, liquid suspension, liquid emulsion, or other suitable nasal dosage form.

The aminosterol composition can also be included in nutraceuticals. For instance, the aminosterol composition may be administered in natural products, including milk or milk product obtained from a transgenic mammal which expresses alpha-fetoprotein fusion protein. Such compositions can also include plant or plant products obtained from a transgenic plant which expresses the amino sterol. The aminosterol can also be provided in powder or tablet form, with or without other known additives, carriers, fillers and diluents. Exemplary nutraceuticals are described in Scott Hegenhart, Food Product Design, December 1993.

G. Exemplary Dosages and Dosing Regimens

Effective dosing regimens can also be clinically established based on the dose required to observe a reduction in hallucinations.

In one embodiment, an effective oral dose generally falls between about 10 mg to about 400 mg, or any amount in-between these two values, e.g., about 11 mg, about 12 mg, about 13 mg, about 398 mg, about 399 mg, or about 400 mg/day. In other embodiments, an effective oral dose of an amino sterol in the methods of the invention is about 10 mg, about 15 mg, about 20 mg, about 25 mg, about 30 mg, about 40 mg, about 50 mg to about 300 mg, about 75 mg to about 200 mg, or about 75 mg to about 125 mg. In one specific embodiment, the amount sufficient to produce a beneficial effect is a daily dosage of about 50 mg, about 75 mg, about 100 mg, about 125 mg, about 150 mg, about 175 mg, or about 200 mg, about 225 mg, about 250 mg, about 275 mg, about 300 mg, about 325 mg, about 350 mg, about 375 mg, or about 400 mg/day.

Dosing can be done on an as needed basis using any pharmaceutically acceptable dosing regimen. For example, dosing can be once or twice daily, once every other day, once every three days, once every four days, once every five days, once every six days, once a week, or divided over multiple time periods during a given day (e.g., twice daily). The dosing schedule may include administration during the morning, midday, or during the evening, or a combination thereof.

In one embodiment, effective dosing regimens can in part be established by measuring the rate of excretion of the orally or nasally administered amino sterol and correlating this with clinical symptoms and signs (i.e., reduction in occurrence of hallucinations). Exemplary dosing regimens include, but are not limited to: Initiating with a “low” initial daily dose, and gradually increasing the daily dose until a dose is reached that minimizes, reduces, or eliminates the hallucinations. In some embodiments, a “low” dose is from about 10 to about 100 mg per person, and the final effective daily dose may be between about 50 to about 1000 mg/person.

Another exemplary dosing regimen includes initiating with a “high” initial dose, which necessarily stimulates the enteric nervous system, and reducing the subsequent daily dosing to that required to elicit a clinically acceptable reduction or elimination of hallucinations, with the “high” daily dose being between about 50 to about 1000 mg/person, and the subsequent lower daily oral dose being between about 10 to about 500 mg/person.

In some embodiments, treatment of hallucinations according to the disclosed methods may prevent or substantially decrease the subsequent development of central nervous system (CNS) disorders including, but not limited to, synucleopathies, Parkinson's disease, Alzheimer's disease, Lewy body disease, dementia with Lewy bodies, Huntington's disease, schizophrenia, multiple sclerosis, degenerative processes associated with aging, dementia of aging, multi-system atrophy, fronto-temporal dementia, autism, progressive nuclear palsy, Guadeloupian Parkinsonism, and spinocerebellar ataxia, Parkinsonism, Amyotorphic Lateral Sclerosis (ALS), Friedreich's ataxia, vascular dementia, spinal muscular atrophy, supranuclear palsy, progressive supranuclear palsy, progressive nuclear palsy, traumatic brain injury, down syndrome, Gaucher's disease, Krabbe's disease (KD), cerebral palsy, and epilepsy.

In some embodiments, a first or initial “large” dose of aminosterol (e.g., squalamine or another amino sterol) can be selected from the group consisting of about 50, about 75, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1025, about 1050, about 1075, about 1100, about 1125, about 1150, about 1175, about 1200, about 1225, about 1250, about 1275, about 1300, about 1325, about 1350, about 1375, about 1400, about 1425, about 1450, about 1475, about 1500, about 1525, about 1550, about 1575, about 1600, about 1625, about 1650, about 1675, about 1700, about 1725, about 1750, about 1775, about 1800, about 1825, about 1850, about 1875, about 1900, about 1925, about 1950, about 1975, or about 2000 mg/day.

In other embodiments of the invention, the second smaller dose of aminosterol (e.g., squalamine) is less than the first or initial dose and can be selected from the group consisting of about, 10, about 25, about 50, about 75, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, or about 1000 mg/day.

Finally, in other embodiments of the invention, the periodic squalamine dosage (per person) can be selected from the group consisting of about 10, about 25, about 50, about 75, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, and about 1000 mg/day.

The pharmaceutical composition comprising an aminosterol or a derivative or salt thereof can be administered for any suitable period of time, including as a maintenance dose for a prolonged period of time. Dosing can be done on an as needed basis using any pharmaceutically acceptable dosing regimen. Aminosterol dosing can be no more than 1× per day, once every other day, once every three days, once every four days, once every five days, once every six days, once a week, or divided over multiple time periods during a given day (e.g., twice daily).

Repeat dosing regimens may be timed by the rate of clearance of the aminosterol from the intestine. It is assumed that at a certain time after the initial “loading” dose, surface concentrations of the aminosterol will decrease as the substance spreads across the surface of the intestinal walls and progresses distally. For example, the aminosterol-induced response appears to last about 4 days following a single 200 mg oral dose of squalamine or Aminosterol 1436. A second dose on day 4 of about 100 mg, followed by successive doses of about 100 mg every 4 days, would represent one reasonable regimen designed to maintain a steady state surface concentration in the intestine. For the purposes of the current methods, daily dosing is the preferable regimen.

In other embodiments, the composition can be administered: (1) as a single dose, or as multiple doses over a period of time; (2) at a maintenance dose for an indefinite period of time; (3) once, twice or multiple times; (4) daily, every other day, every 3 days, weekly, or monthly; (5) for a period of time such as about 1, about 2, about 3, or about 4 weeks, about 1, about 2, about 3, about 4, about 5, about 6, about 7, about 8, about 9, about 10, about 11, or about 12 months, about 1 year, about 1.5 years, about 2, about 2.5, about 3, about 3.5, about 4, about 4.5, about 5, about 5.5, about 6, about 6.5, about 7, about 7.5, about 8, about 8.5, about 9, about 9.5, about 10, about 10.5, about 11, about 11.5, about 12, about 12.5, about 13, about 13.5, about 14, about 14.5, about 15, about 15.5, about 16, about 16.5, about 17, about 17.5, about 18, about 18.5, about 19, about 19.5, about 20, about 20.5, about 21, about 21.5, about 22, about 22.5, about 23, about 23.5, about 24, about 24.5, or about 25 years, or (6) any combination of these parameters, such as daily administration for 6 months, weekly administration for 1 or more years, etc.

Yet another exemplary dosing regimen includes periodic dosing, where an effective dose can be delivered once every about 1, about 2, about 3, about 4, about 5, about 6 days, or once weekly, with the initial dose determined to be capable of eliciting a response that abolishes hallucinations.

Aminosterol dosing should continue at least until the clinical condition has resolved. To establish the need for continued dosing, treatment can be discontinued and the condition revaluated. If necessary, aminosterol administration should be resumed. The period of oral dosing can be for about 1, about 2, about 3, or about 4 weeks; about 1, about 2, about 3, about 4, about 5, about 6, about 7, about 8, about 9, about 10, about 11, or about 12 months; or about 1, about 2, about 3, about 4, or 5 years, or longer.

Optimal oral dosing appears to be on an empty stomach. Squalamine, for example, is expected to bind tightly to foodstuff, and be unavailable to interact with the intestinal epithelium. Only as the food material is digested is squalamine freed. Such would be occurring in the more distal intestine.

In a preferred embodiment, the aminosterol dose is taken in the morning, i.e. on an empty stomach preferably within about two hours of waking up and may be followed by a period without food, such as for example about 60 to about 90 minutes. In other embodiments, the aminosterol dose is taken within about 15 min, about 30 min, about 45 min, about 1 hr, about 1.25 hrs, about 1.5 hrs, about 1.75 hrs, about 2 hrs, about 2.25 hrs, about 2.5 hrs, about 2.75 hrs, about 3 hrs, about 3.25 hrs, about 3.5 hrs, about 3.75 hrs, or about 4 hrs within waking up. In yet further embodiments, the aminosterol dose is followed by about period without food, wherein the period is at least about 30 min, about 45 mins, about 60 mins, about 1.25 hrs, about 1.5 hrs, about 1.75 hrs, or about 2 hrs.

Not to be bound by theory, it is believed that since aminosterols have an impact on circadian rhythms, likely due to ENS signaling thereof, taking the amino sterol dose in the morning enables the synchronization of all the autonomic physiological functions occurring during the day. In other embodiments of the invention, the amino sterol dosage is taken within about 15 mins, about 30 mins, about 45 mins, about 1 hour, about 1.25 hrs, about 1.5 hrs, about 1.75 hrs, about 2 hrs, about 2.25 hrs, about 2.5 hrs, about 2.75 hrs, about 3 hrs, about 3.25 hrs, about 3.5 hrs, about 3.75 hrs, or about 4 hrs of waking up. In addition, in other embodiments of the invention, following the aminosterol dosage the subject has a period of about 15 mins, about 30 mins, about 45 mins, about 1 hours, about 1.25 hrs, about 1.5 hrs, about 1.75 hrs, about 2 hrs, about 2.25 hrs, about 2.5 hrs, about 2.75 hrs, or about 3 hours without food.

Failure to elicit an aminosterol-induced reduction in hallucinations would generally suggest that the dose being administered was inadequate, and would suggest continued titration until the desired reduction in hallucination is observed or the subject is free of hallucinations. An effective dose can be considered a dose which induces the desired reduction in hallucination or results in that the subject is hallucination free.

The sensitivity of the aminosterol-induced reduction in hallucination following administration of amino sterols is likely due to several variables: (1) the absorption of the aminosterol into a mucous layer, an effect that would reduce free concentration of aminosterol available for diffusion onto the epithelial surface, thereby reducing the response to a given oral dose; and (2) an increase in the permeability of the epithelial wall (leakiness), which occurs following infections, allergic enteropathies, and in states of intestinal inflammation. In such settings, the normal transport of the amino sterol across the epithelium, which is facilitated by the controlled entry and subsequent exit of the molecule from the lining epithelial cell, would be circumvented. The compound would leak across the epithelial barrier, and expose the nerve network within the bowel wall to abnormally high concentrations. Hence, an excessive response might provide a diagnostic impression of the permeability status of the epithelium.

The disclosed methods can be used to treat a range of subjects, including human and non-human animals, including mammals, as well as immature and mature animals, including human infants, toddlers, children, adults, and elderly.

Any pharmaceutical used for therapeutic administration can be sterile. Sterility is readily accomplished by for example filtration through sterile filtration membranes (e.g., 0.2 micron membranes). Any pharmaceutically acceptable sterility method can be used in the compositions of the invention.

H. Kits

Aminosterol formulations or compositions of the invention may be packaged together with, or included in a kit along with instructions or a package insert. Such instructions or package inserts may address recommended storage conditions, such as time, temperature and light, taking into account the shelf-life of the aminosterol or derivatives or salts thereof. Such instructions or package inserts may also address the particular advantages of the aminosterol or derivatives or salts thereof, such as the ease of storage for formulations that may require use in the field, outside of controlled hospital, clinic or office conditions.

The invention also provides a pharmaceutical pack or kit comprising one or more containers filled with one or more amino sterol pharmaceutical compositions disclosed herein. The kits may include, for instance, containers filled with an appropriate amount of an aminosterol pharmaceutical composition, either as a powder, a tablet, to be dissolved, or as a sterile solution. Associated with such container(s) can be a notice in the form prescribed by a governmental agency regulating the manufacture, use or sale of pharmaceuticals or biological products, which notice reflects approval by the agency of manufacture, use or sale for human administration. In addition, the aminosterol or a derivative or salt thereof may be employed in conjunction with other therapeutic compounds.

In other aspects, a kit comprising a nasal spray device as described herein is disclosed. In one aspect, the kit may comprise one or more devices as disclosed herein, comprising a disclosed low dose aminosterol composition, wherein the device is sealed within a container sufficient to protect the device from atmospheric influences. The container may be, for example, a foil, or plastic pouch, particularly a foil pouch, or heat sealed foil pouch. Suitable containers sufficient to adequately protect the device will be readily appreciated by one of skill in the art.

In one aspect, the kit may comprise one or more devices as disclosed herein, wherein the device may be sealed within a first protective packaging, or a second protective packaging, or a third protective packaging, that protects the physical integrity of the product. One or more of the first, second, or third protective packaging may comprise a foil pouch. The kit may further comprise instructions for use of the device. In one aspect, the kit contains two or more devices.

In one aspect, the kit may comprise a device as disclosed herein, and may further comprise instructions for use. In one aspect, the instructions may comprise visual aid/pictorial and/or written directions to an administrator of the device.

I. Patient Populations

The disclosed compositions can be used to treat a range of subjects, including human and non-human animals, including mammals, as well as immature and mature animals, including human children and adults. The human subject to be treated can be an infant, toddler, school-aged child, teenager, young adult, adult, or elderly patient.

In embodiments disclosed herein relating to prevention, particular patient populations may be selected based on being “at risk for” the development of one or more disorders. For example, genetic markers of hallucination associated diseases, such as PD (e.g., SNCA (PARK1), UCHL1 (PARK 5), and LRRK2 (PARK8)) or family history may be used as signs to identify subjects likely to develop hallucinations. Thus, in some embodiments relating to disorders for which certain genetic or hereditary signs are known, prevention may involve first identifying a patient population based on one of the signs. Alternatively, certain symptoms are considered early signs of particular disorders. Thus, in some embodiments, a patient population may be selected for being “at risk” for developing hallucinations based on age and experiencing constipation. Further genetic or hereditary signs may be used to refine the patient population.

IV. Methods of Treating Hallucinations and/or a Hallucination-Related Condition or Disease with a “Fixed Dose” of Aminosterol

Aspects of this disclosure relate to methods of treating, preventing, and/or delaying the onset or progression of hallucinations and/or a hallucination-related condition by administration of a “fixed dose” of an aminosterol as disclosed herein. The hallucinations can be correlated with abnormal α-synuclein (αS) pathology. Alternatively, the hallucinations can be correlated dysfunctional DA neurotransmission, also known as dopaminergic dysfunction.

This disclosure provides a detailed protocol for determining a “fixed dose” based on improvement of one symptom associated with Parkinson's disease (PD), e.g., hallucinations and hallucination-related symptoms as measured by clinically recognized scales and tools.

As dopaminergic activity distinguishes PD from other neurodegenerative disorders and these data relate to symptoms that do not relate to this distinguishing feature, this dosing regime is believed to be extrapolatable both hallucinations per se and hallucination related symptoms.

Not to be bound by theory, it is believed that establishing a patient-specific “fixed dose” based on obtaining a threshold improvement in any of the hallucination-related symptoms described herein will successfully treat hallucinations and/or hallucination related symptoms. Further, to the extent that these symptoms are tied to an underlying disorder, administration of the therapeutically effective fixed dose is also believed to offer a means of treating, preventing, and/or delaying onset of the underlying disorder or disease causing the hallucination or hallucination-related symptom.

A. Hallucinations

A hallucination is a sensory impression or perception of an object or event, in any of the five senses (sight, touch, sound, smell, or taste) that has no basis in external stimulation. Examples of hallucinations include “seeing” someone not there (visual hallucination), “hearing” a voice not heard by others (auditory hallucination), “feeling” something crawling up your leg (tactile hallucination), “smelling” (olfactory), and “tasting” (gustatory). Other examples of hallucination types include hypnagogic hallucination (a vivid, dreamlike hallucination occurring at sleep onset), hypnopompic hallucination (a vivid, dreamlike hallucination occurring on awakening), kinesthetic hallucination (a hallucination involving the sense of bodily movement), and somatic hallucination, a hallucination involving the perception of a physical experience occurring within the body.

A paracusia, or auditory hallucination, is a form of hallucination that involves perceiving sounds without auditory stimulus. Tactile hallucination is the false perception of tactile sensory input that creates a hallucinatory sensation of physical contact with an imaginary object. An olfactory hallucination (phantosmia) makes an individual detect smells that aren't really present in their environment.

Hallucinations can be psychiatric condition related. Hallucinations, especially auditory hallucinations, are characteristic of certain psychiatric conditions such as schizophrenia, occurring in up to 70-80% of subjects. They also occur in 30-50% of individuals with borderline personality disorder. They can also occur in post-partum psychosis. Auditory hallucinations can be related to severe depression or mania. Substance abuse disorder (SAD) can also be hallucination related condition. Alcohol intoxication or withdrawal, post-traumatic stress disorder (PTSD) and bereavement hallucination related conditions.

Hallucinations can be related to neurological disorders. The neurological disorder can be caused by brain tumors. The neurological disorder can be caused by sleep disorders such as narcolepsy. Furthermore, neurological disorders may be a variety of focal brain lesions, resulting in particular types of hallucinations depending on the location on the lesion.

Hallucinations may be related to diffuse involvement of the cerebral cortex. In some embodiments, the diffuse involvement of the cerebral cortex may be caused by a viral infectious disease. In other embodiments, the diffuse involvement of the cerebral cortex may be a result of a cerebral vasculitis condition. The cerebral vasculitis condition can be caused by autoimmune disorders, bacterial or viral infection, or systemic vasculitis.

Hallucinations can be related to neurodegenerative disorders, including for example synucleopathies, Parkinson's disease, Alzheimer's disease, dementia with Lewy bodies (DLB), multiple system atrophy (MSA), Huntington's Disease, Multiple Sclerosis (MS), Amyotorphic Lateral Sclerosis (ALS), schizophrenia, Friedreich's ataxia, vascular dementia, spinal muscular atrophy, supranuclear palsy, fronto temperal dementia (FTD), progressive supranuclear palsy, Guadeloupian Parkinsonism, Parkinsonism, spinocerebellar ataxia, autism, stroke, traumatic brain injury, sleep disorders such as REM sleep behavior disorder (RBD), depression, down syndrome, Gaucher's disease (GD), Krabbe's disease (KD), lysosomal conditions affecting glycosphingolipid metabolism, ADHD, agitation, anxiety, delirium, irritability, illusion and delusions, amnesia, apathy, bipolar disorder, disinhibition, aberrant motor and obsessive-compulsive behaviors, addiction, cerebral palsy, epilepsy, major depressive disorder, degenerative processes associated with aging, and dementia of aging.

Hallucinations can be related to neurological disorders such as, for example, (a) a brain tumor, (b) a sleep disorder such as narcolepsy or REM sleep behavior disorder (RBD), or (c) a focal brain lesion, such as occipital lobe lesions or temporal lobe lesions. The neurological disorder can be, for example, the result of (d) a diffuse involvement of the cerebral cortex, such as that caused by a viral infectious disease. For example, the viral infectious disease can be selected from the group consisting of acute metabolic encephalopathies, encephalitis, and meningitis. In another embodiment, the diffuse involvement of the cerebral cortex is a result of a cerebral vasculitis condition. For example, the cerebral vasculitis condition can be caused by an autoimmune disorder, a bacterial or viral infection, or a systemic vasculitis. For example, the autoimmune disorder can be Systemic Lupus Erythematosus (SLE).

Hallucinations can be related to psychiatric disorders such as, for example, bipolar disorder, borderline personality disorder, depression, depression (mixed), dissociative identity disorder, generalized anxiety disorder, major depression, major depressive disorder, obsessive compulsive disorder, aberrant motor and obsessive-compulsive behaviors, addiction, post-traumatic stress disorder, psychosis (NOS), schizoaffective disorder, ADHD, agitation, anxiety, delirium, irritability, illusion and delusions, amnesia, apathy, and schizophrenia. Hallucinations can be related to borderline dementia.

Hallucinations can be related to sensory loss. Progressive visual loss and blindness can be associated with visual hallucinations (Charcot-Bonnet syndrome) and is exacerbated by dim light. Hallucinations related to sensory loss can be simple or complex. Hallucinations have also been reported in individuals with congenital blindness. Auditory hallucinations can occur in individuals with hearing loss and deafness and can be unilateral or bilateral. Hallucinations can also be related to congenital deafness.

Hallucinations can be related to dysfunction of the enteric nervous system. In some embodiments the hallucination related condition is a synucleinopathy. In some embodiments the hallucination related condition is α-synuclein deposition.

In one embodiment, the method results in a positive impact or improvement in hallucinations or a hallucination-related condition, wherein the positive impact or improvement is measured using a medically recognized technique, and the improvement is about 3%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In some embodiments, the medically recognized technique is selected from the group consisting of Chicago Hallucination Assessment Tool (CHAT), The Psychotic Symptom Rating Scales (PSYRATS), Auditory Hallucinations Rating Scale (AHRS), Hamilton Program for Schizophrenia Voices Questionnaire (HPSVQ), Characteristics of Auditory Hallucinations Questionnaire (CAHQ), Mental Health Research Institute Unusual Perception Schedule (MUPS), positive and negative syndrome scale (PANSS), scale for the assessment of positive symptoms (SAPS), Launay-Slade hallucinations scale (LSHS), the Cardiff anomalous perceptions scale (CAPS), and structured interview for assessing perceptual anomalies (SIAPA).

I. Neurodegenerative Diseases and Neurological Diseases Associated with Neural Cell Death

The methods and compositions of the invention may also be useful in treating, preventing, and/or delaying the onset or progression of hallucinations correlated with abnormal αS pathology, and/or dysfunctional DA neurotransmission, wherein the underlying hallucination-related condition is a neurodegenerative disease or neurological disorder. Examples of such neurodegenerative diseases or neurological disorders include, but are not limited to, PD, AD, LBD, FTD, supranuclear palsy, MSA, Parkinsonism, ALS, Huntington's Disease, schizophrenia, Friedreich's ataxia, MS, spinal muscular atrophy, progressive nuclear palsy, degenerative processes associated with aging, dementia of aging, Guadeloupian Parkinsonism, spinocerebellar ataxia, and vascular dementia.

In addition, the methods and compositions of the invention may also be useful in treating, preventing, and/or delaying the onset or progression of hallucinations correlated with abnormal αS pathology, and/or dysfunctional DA neurotransmission, wherein the underlying hallucination-related condition is a neurological disease associated with neural cell death and/or related symptoms of neural cell death such as septic shock, intracerebral bleeding, subarachnoidal hemorrhage, multiinfarct dementia, inflammatory diseases, neurotrauma, peripheral neuropathies, polyneuropathies, epilepsies, schizophrenia, depression, metabolic encephalopathies, or infections of the central nervous system.

A variety of neuroimaging techniques may be useful for the early diagnosis and/or measurement of progression of neurodegenerative disorders that are hallucination-related. Examples of such techniques include but are not limited to neuroimaging, functional MRI, structural MRI, diffusion tensor imaging (DTI) (including for example diffusion tensor measures of anatomical connectivity), [18F]fluorodeoxyglucose (FDG) PET, agents that label amyloid, [18F]F-dopa PET, radiotracer imaging, volumetric analysis of regional tissue loss, specific imaging markers of abnormal protein deposition (e.g., for depression associated disease such as AD and PD), multimodal imaging, and biomarker analysis (Jon Stoessl, 2012). Combinations of these techniques can also be used to measure disease progression. For example, structural MRI can be used to measure atrophy of the hippocampus and entorhinal cortex in AD, as well as involvement of the lateral parietal, posterior superior temporal and medial posterior cingulate cortices. DTI can be used to show abnormal white matter in the parietal lobes of patients with dementia with Lewy bodies (DLB) as compared to AD. Functional MRI may reveal reduced frontal but increased cerebellar activation during performance of a working memory task in FTD compared to AD. In another example, [18F]fluorodeoxyglucose (FDG) PET can show reduced glucose metabolism in parietotemporal cortex in AD. An electroencephalogram (EEG) can be used as a biomarker for the presence and progression of a neurodegenerative disease.

i. Parkinson's Disease

PD is the second most common age-related neurodegenerative disease after AD (Reeve et al. 2014). PD affects over 1% of the population over the age of 60, which in the US equates to over 500,000 individuals, while in individuals over the age of 85 this prevalence reaches 5%, highlighting the impact that advancing age has on the risk of developing this condition. Id.

While motor symptoms are still required for a diagnosis of PD (Hughes et al. 1992), non-motor symptoms represent a greater therapeutic challenge (Zahodne et al. 2012). These symptoms include hallucinations (Friedman et al. 2018; Diederich et al. 2009), cognitive dysfunction (Auyeung et al. 2012), as well as constipation (Ondo et al. 2012; Lin et al. 2014), disturbances in sleep architecture (Ondo et al. 2001; Gjerstad et al. 2006), REM behavior disorder (RBD) and depression (Aarsland et al. 2007), all of which result from impaired function of neural pathways not restored by replacement of dopamine. In fact, long-term institutionalization, caregiver burden and decrease in life expectancy correlate more significantly with the severity of these symptoms than with motor symptoms (Goetz et al. 1993).

PD is a progressive neurodegenerative disorder caused by accumulation of the protein αS within the ENS, autonomic nerves and brain (Braak et al. 2003). In 2003, Braak proposed that PD begins within the GI tract caused when neurotoxic aggregates of αS form within the ENS, evidenced clinically by the appearance of constipation in a majority of people with PD many years before the onset of motor symptoms. A recent study in rats has demonstrated movement of aggregates of αS from the ENS to the CNS via the vagus and other afferent nerves. Neurotoxic aggregates accumulated progressively within the brainstem and then dispersed rostrally to structures within the diencephalon, eventually reaching the cerebral hemispheres.

PD is defined as a synucleinopathy, and synuclein deposition remains the main final arbiter of diagnosis. Additionally, patients with dementia and Lewy bodies are considered as having PD if they meet clinical disease criteria. Imaging (e.g., MRI, single photon emission computed tomography [SPECT], and positron emission tomography [PET]) allows in vivo brain imaging of structural, functional, and molecular changes in PD patients.

There has been research in the last few years identifying particular markers or combinations of markers that are used for probabilistic estimates of prodromal PD. Researchers have identified a timeline of symptoms indicative of prodromal PD and predictive of PD. The presence of each contributes to an estimate of the likelihood of prodromal PD. Some have been adopted for identification of prodromal PD. Other studies use a combination of symptoms and imaging (e.g., hyposmia combined with dopamine receptor imaging has been found to have a high predictive value). In another example, REM sleep behavior disorder (SBD), constipation, and hyposmia were found to be individually common but to rarely co-occur in individuals without PD, leading to a high predictive value for PD. Thus, patient populations having RBD, constipation, and/or hyposmia are considered at risk for developing PD.

Data described in Example 4 shows remarkable improvement in a wide variety of symptoms correlated with PD, including a significant and positive effect on hallucinations. The study demonstrates that administration of an aminosterol can displace αS from membranes in vitro and reduce the formation of neurotoxic αS aggregates in vivo, thereby improving related hallucinations. The study is the first proof of concept demonstration that directly targeting αS pharmacologically can achieve beneficial GI, autonomic and CNS responses to improve hallucination in patients suffering from neurodiseases such as PD. These results demonstrate that the ENS in PD is not irreversibly damaged and can be restored to normal function.

As described in Example 4, CNS symptoms were evaluated at baseline and at the end of the fixed dose period and the wash-out period (Table 12). Moreover, the improvement in many CNS symptoms persisted during wash-out. The results of treatment were dramatic: MMSE (cognitive ability) improved from 28.4 at baseline to 28.7 during treatment and to 29.3 during wash-out. Other symptoms evaluated and showing improvement included:

(1) Total UPDRS score was 64.4 at baseline, 60.6 at the end of the fixed dose period and 55.7 at the end of the wash-out period; similarly, the motor component of the UPDRS improved from 35.3 at baseline to 33.3 at the end of fixed dose to 30.2 at the end of wash-out. The UPDRS score, a global assessment of motor and non-motor symptoms, showed significant improvement. Improvement was also seen in the motor component. The improvement in the motor component is unlikely to be due to improved gastric motility and increased absorption of dopaminergic medications, since improvement persisted during the 2-week wash-out period, i.e., in the absence of study drug (Table 12).

(2) BDI-II (depression) decreased from 10.9 at baseline to 9.9 during treatment and 8.7 at wash-out.

(3) PDHQ (hallucinations) improved from 1.3 at baseline to 1.8 during treatment and 0.9 during wash-out. Hallucinations were reported by 5 patients at baseline and delusions in 1 patient. Both hallucinations and delusions improved or disappeared in 5 of 6 patients during treatment and did not return for 4 weeks following discontinuation of aminosterol treatment in 1 patient and 2 weeks in another. In one patient the hallucinations disappeared at 100 mg, despite not having reached the colonic prokinetic dose at 175 mg.

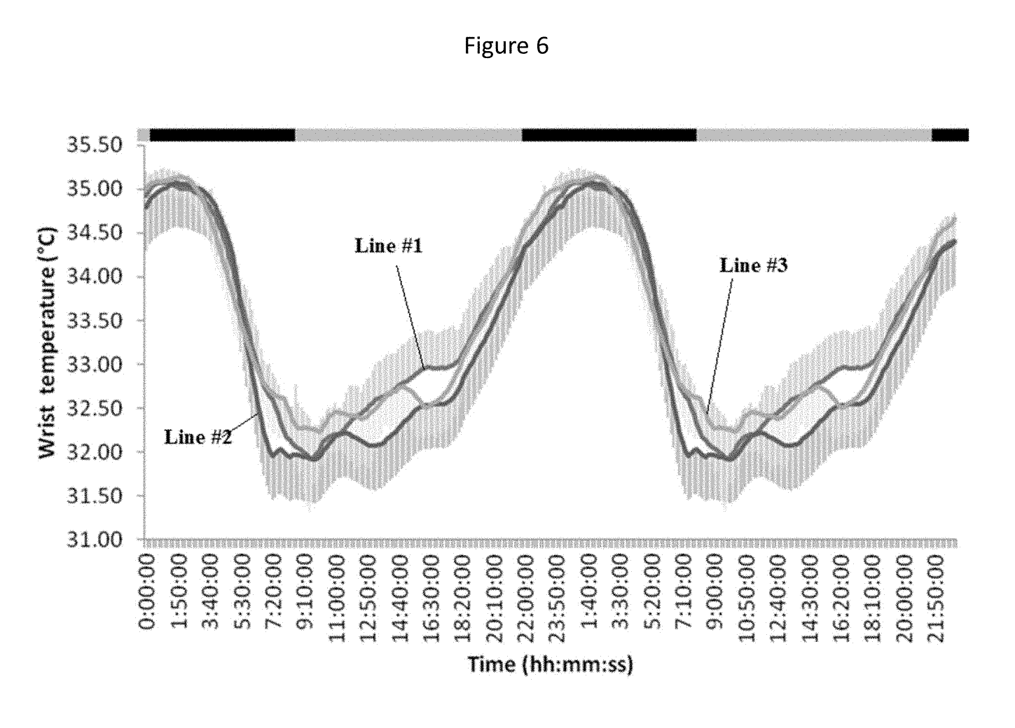

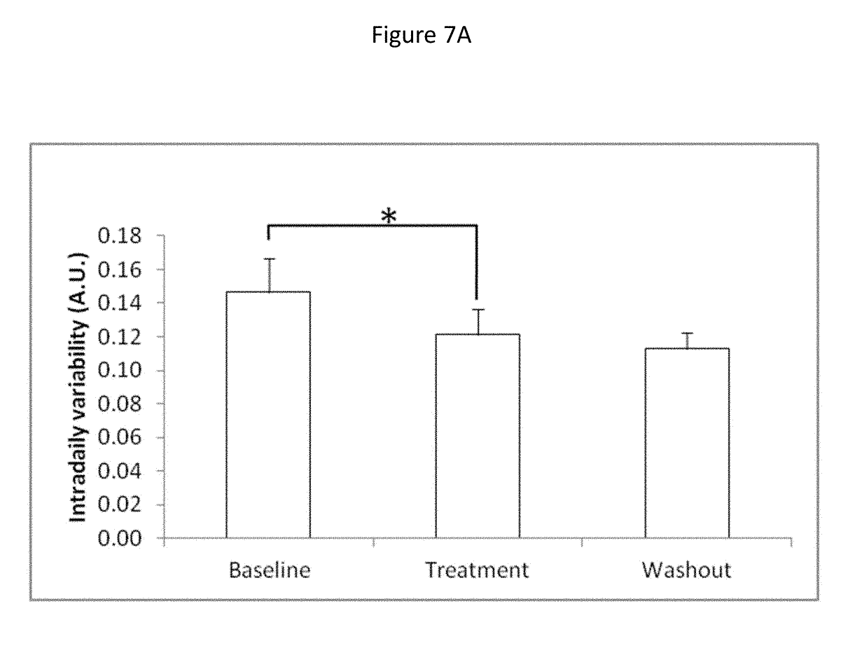

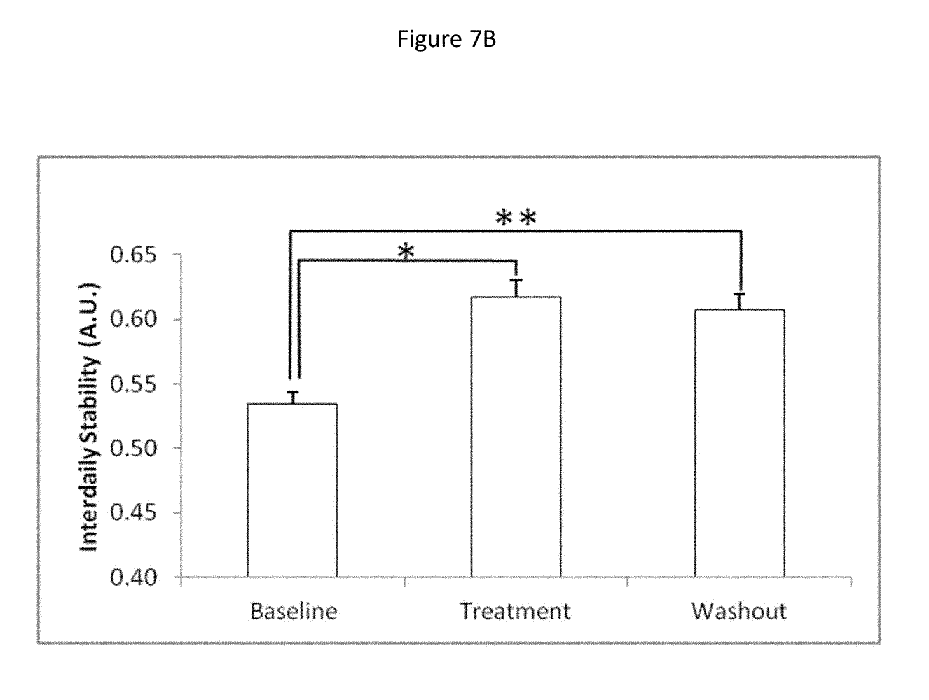

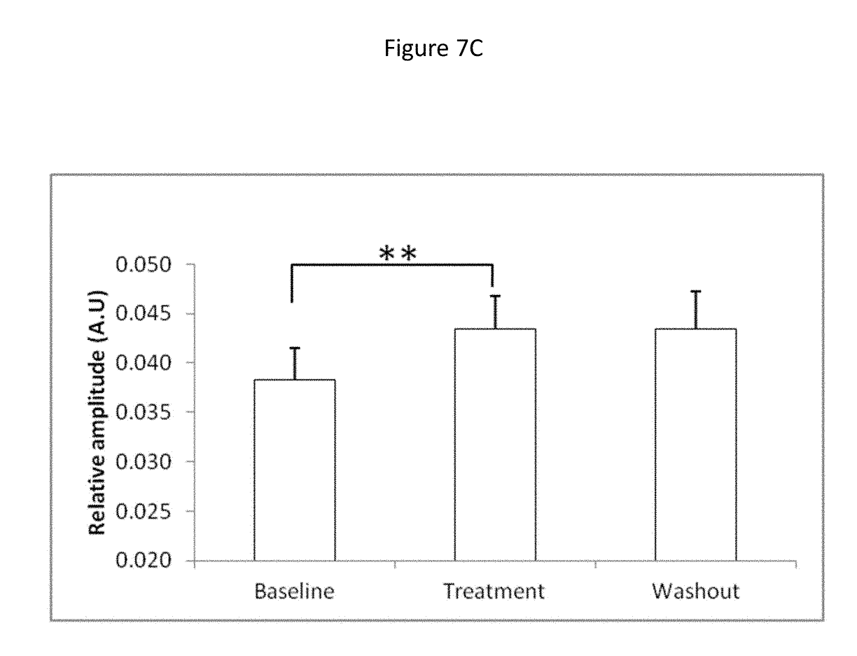

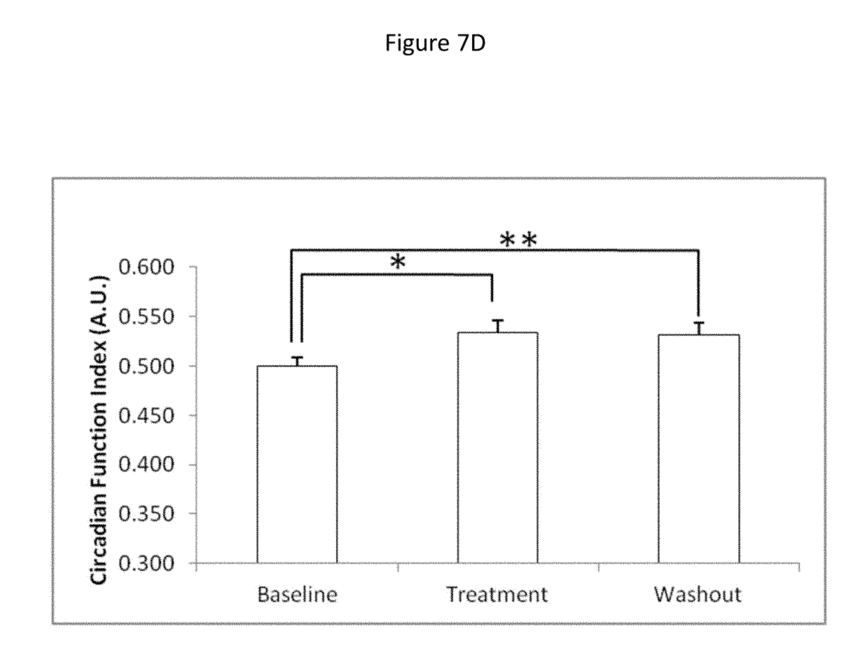

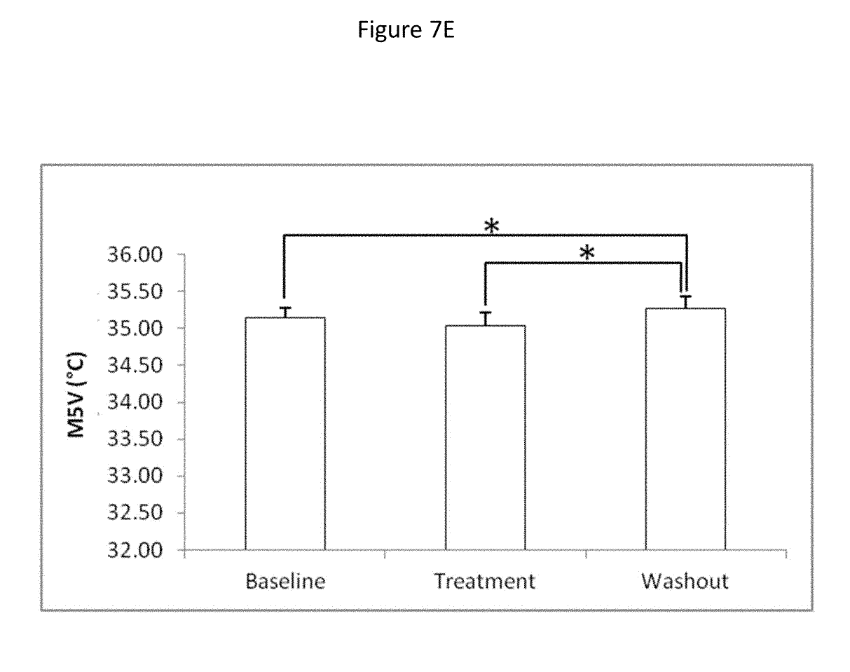

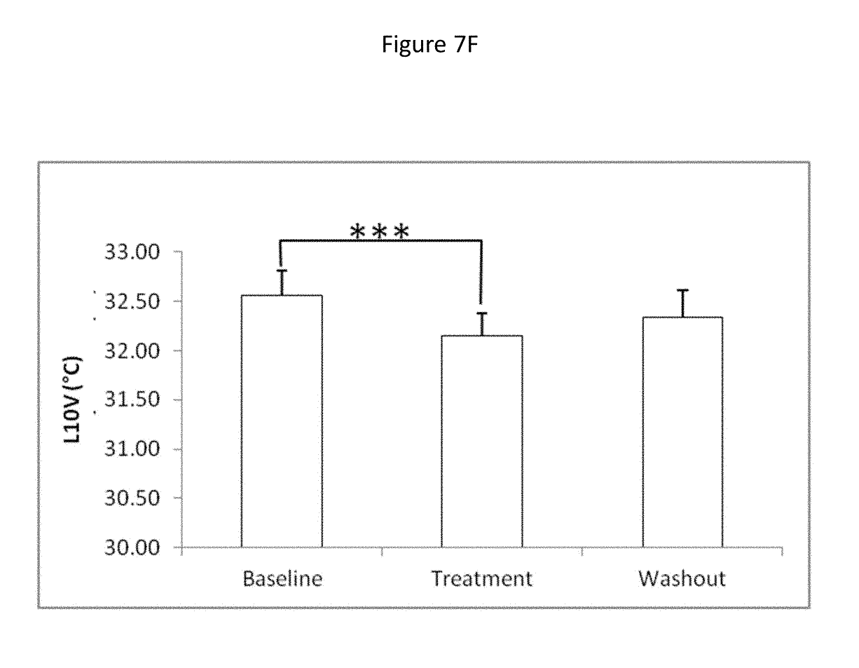

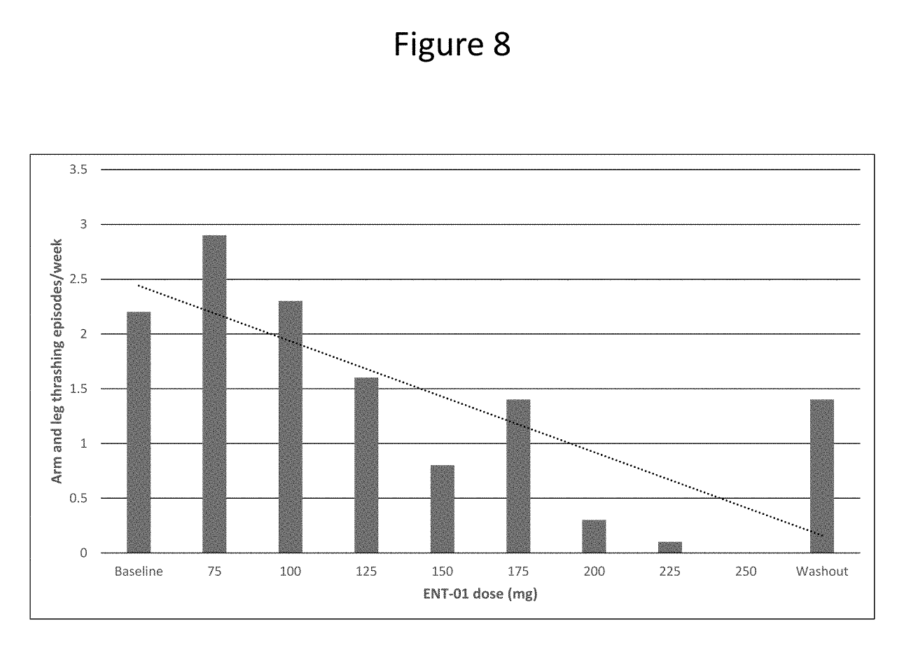

(4) Improvements were seen in REM-behavior disorder (RBD) and sleep. RBD and total sleep time also improved progressively in a dose-dependent manner. The frequency of arm or leg thrashing reported in the sleep diary diminished progressively from 2.2 episodes/week at baseline to 0 at maximal dose. Total sleep time increased progressively from 7.1 hours at baseline to 8.4 hours at 250 mg and was consistently higher than baseline beyond 125 mg (FIGS. 6-8).

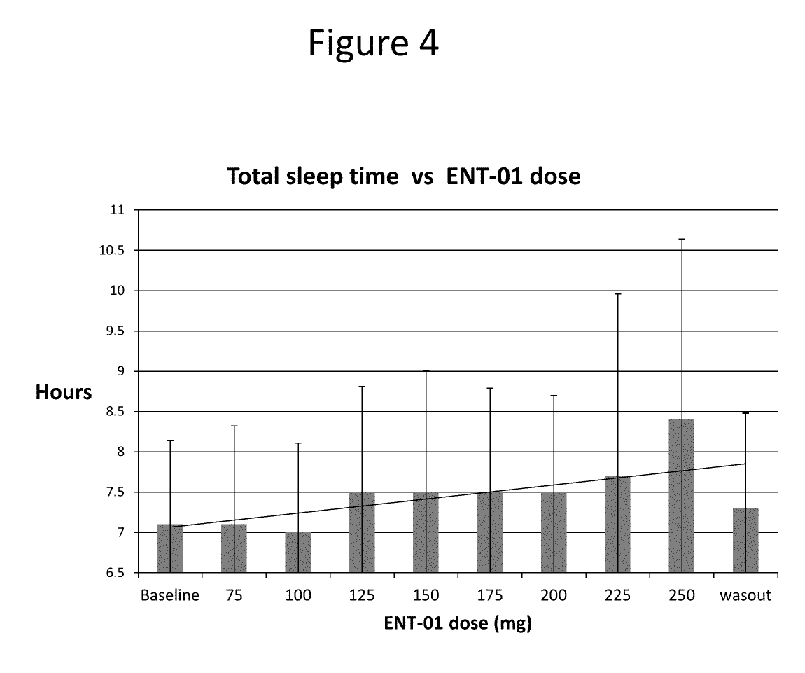

Example 4 describes calibration of a fixed amino sterol dose for a specific PD patient using constipation as the symptom or marker by which improvement was measured. In Example 4, the degree of constipation was measured by the number of complete spontaneous bowel movement (CSBM) or spontaneous bowel movement (SBM) per week, with an increase in the number of CSBM or SBM per week demonstrating a desired escalated aminosterol dose. Data detailed in Example 4 shows that 80% of subjects responded to aminosterol treatment with improved bowel function (see FIG. 4A), with the cumulative response rate increasing in a dose-dependent fashion from 25% at 25 mg to a maximum of 80% at 200 mg (Stage 1, FIG. 4A). In Stage 2 of the study, the response rate increased in a dose-dependent fashion from 26% at 75 mg to 85.3% at 250 mg (FIG. 4A). The dose required for a bowel response was patient-specific and varied from 75 mg to 250 mg. The median efficacious dose was 100 mg. The dose that proved efficacious in inducing a bowel response was strongly related to constipation severity at baseline (FIG. 4B); patients with baseline constipation of <1 CSBM/week required higher doses for a response (mean 192 mg) than patients with ≥1 CSBM/week (mean 120 mg). Thus, the severity of constipation correlates with a higher required “fixed escalated amino sterol dose.”

The observation that the aminosterol dose required to achieve a desired response increases with symptom severity supports the hypothesis that the greater the burden of αS impeding neuronal function, the higher the dose of aminosterol required to restore normal function and improve or resolve the symptom. It is theorized that the aminosterol dose required to obtain a positive effect in a subject for the symptom being evaluated correlates with the extent of neuronal damage. Thus, it is theorized that greater neuronal damage correlates with a higher required aminosterol dose to obtain a positive effect in a subject for the symptom being evaluated. For examples, the symptom to be evaluated may be any one of the symptoms detailed herein for hallucinations and the medically recognized techniques described herein may be used for measuring improvement in hallucination symptoms to calibrate the aminosterol dosage for a particular patient.

In calibrating the fixed aminosterol dose for a specific patient, the starting dose is varied based upon the severity of the hallucinations. Thus, for subjects with severe hallucinations based on a medically recognized technique, oral aminosterol dosing is started at from about 75 to about 175 mg/day mg or more (or any amount in-between these values as described herein). For subjects with mild or hallucinations based on a baseline score on medically recognized technique that correlates with mild or moderate hallucinations, oral aminosterol dosing is started at about 1 to about 75 mg/day (or any amount in-between these values as described herein). Dosing for both patients is then escalated by defined amounts over a defined period of time until the fixed escalated dose for the patient is identified.

ii. Alzheimer's Disease (AD), MSA, and Schizophrenia

Other hallucination-related conditions or disorders that are correlated with abnormal α-synuclein (αS) pathology, and/or dysfunctional DA neurotransmission, also known as dopaminergic dysfunction, are described above in Section I B. and include, for example, AD, MSA, and Schizophrenia.

There are currently a variety of art-accepted methods for diagnosing probable AD. Typically, these methods are used in combination. These methods include determining an individual's ability to carry out daily activities and identifying changes in behavior and personality. Hallucinations have a prevalence in Alzheimer's disease of 4% to 76% (median=23%) (Bassiony et al. 2003).

The criteria for ‘probable Alzheimer's disease’ are described a National Institute of Aging-Alzheimer's Association workgroup (McKhann et al. 2011). According to this workgroup, for people who first exhibit the core clinical characteristics of AD dementia, evidence of biomarkers associated with the disease may enhance the certainty of the diagnosis.

Multiple system atrophy (MSA) is a progressive neurodegenerative disorder characterized by a combination of symptoms that affect both the autonomic nervous system and movement. This is caused by progressive degeneration of neurons in several parts of the brain including the substantia nigra, striatum, inferior olivary nucleus, and cerebellum. There is no known cure for MSA and management is primarily supportive.

Hallucinations, especially auditory hallucinations, are characteristic of schizophrenia, occurring in up to 70-80% of subjects (Yee et al., 2005). Schizophrenia is a chronic progressive disorder that has at its origin structural brain changes in both white and gray matter. It is likely that these changes begin prior to the onset of clinical symptoms in cortical regions, particularly those concerned with language processing. Later, they can be detected by progressive ventricular enlargement. Current magnetic resonance imaging (MRI) technology can provide a valuable tool for detecting early changes in cortical atrophy and anomalous language processing, which may be predictive of who will develop schizophrenia.

A 2013 study of schizophrenia patients documented brain changes seen in MRI scans from more than 200 patients beginning with their first episode and continuing with scans at regular intervals for up to 15 years. The scans showed that people at their first episode had less brain tissue than healthy individuals. The findings suggest that those who have schizophrenia are being affected by something before they show outward signs of the disease.

While not wishing to be bound by theory, it is theorized that administration of a therapeutically effective fixed dose of an aminosterol composition to a schizophrenia patient may treat and/or prevent hallucination-related symptoms associated with schizophrenia. In some embodiments, the administration may be oral—resulting in absorption in the ENS. In some embodiments, the administration may be intranasal—resulting in stimulation of neurogenesis, which has a positive impact on the loss of brain tissue characteristic of schizophrenia subjects.

iii. Other Neurodegenerative Disorders

The methods and compositions of the invention may also be useful in treating, preventing, and/or slowing the onset or progression of hallucinations correlated with abnormal α-synuclein (αS) pathology, and/or dysfunctional DA neurotransmission, also known as dopaminergic dysfunction, where the underlying condition is a variety of other neurodegenerative disorders. Examples are given above in Section I.B., and include but are not limited to, Huntington's disease (HD), progressive supranuclear palsy, Frontotemporal dementia, vascular dementia, also known as multi-infarct dementia (MID) and vascular cognitive impairment (VCI), ALS, MS, SMA, and Friedreich's ataxia.

2. Psychological or Behavioral Disorders

The methods and compositions of the invention may also be useful in treating, preventing, and/or slowing the onset or progression of hallucinations correlated with abnormal αS pathology, and/or dysfunctional DA neurotransmission, also known as dopaminergic dysfunction, where the underlying condition is a psychological or behavioral disorder. Examples are given above in Section I.B as well as below, and include but are not limited to, agitation, anxiety, delirium, irritability, illusion and delusions, amnesia, autism, apathy, bipolar disorder, disinhibition, aberrant motor and obsessive-compulsive behaviors, or sleep disorders.

i. Sleep Problem, Disturbance or Disorder Associated with Hallucinations (e.g., REM Disturbed Sleep or Circadian Rhythm Dysfunction)

Sleep disturbances can be associated with hallucinations. Normal sleep is critically important for the proper functioning of many organ systems, the most important of which is the brain. Disturbances in normal sleep patterns are closely associated with the normal aging process, with impaired memory deposition and consolidation and with the occurrence of neurodevelopmental, neuroaffective and neurodegenerative disorders. The alternating pattern of sleep and wakefulness occurring every 24 hours is known as the circadian rhythm. The rhythm is set by the “Zeitgeber” (time setter), an entity known as the suprachiasmatic nucleus (SCN) and located in the hypothalamus. The SCN is normally “entrained” or synchronized by the external light-dark cycle. This relationship between external light and dark and the sleep wake cycle synchronized to it by the SCN can be over ridden during periods of hunger by neural signals emanating in the gut and relayed to the hypothalamus. The circadian sleep-wake cycle can also shift in response to changes in external light-dark cycles, such as the desynchronization that occurs during travel from one time zone to another (jet-lag). Under such circumstances, a progressive adjustment occurs until the SCN is resynchronized with the external light-dark cycle. A similar “phase-shift” and adjustment occurs in night-shift workers.

Under normal circumstances, the properly functioning SCN, synchronized to the external light-dark cycle and to neural signals emanating from the enteric nervous system, will regulate the sleep-wake cycle by sending neural and chemical signals to the surrounding structures and to portions of the brain stem involved in sleep and wakefulness. An individual with a properly functioning hypothalamus and brain stem will go to bed and fall asleep within minutes, remain asleep throughout the night, wake up in the morning and remain awake and alert throughout the day. During the night, the asleep individual will experience several cycles of sleep, beginning with light sleep, progressing through rapid eye movement sleep (REM-sleep) to deep sleep and back. Each complete sleep period lasts about 90 minutes. Periods of REM-sleep are closely associated with dreaming. During REM-sleep, neural signals emanating from certain parts of the brain stem ensure that skeletal muscles become “atonic” or are paralyzed, such that the individual can't “act out” their dreams.

Certain diseases and conditions may impair the normal functioning of the “Zeitgeber” or circadian clock, for example, disease associated with hallucinations, such as PD. These conditions may be reversible, such as desynchronization resulting from PD. In contrast, damage to the nerves carrying light-dark related information from the retina to the SCN (conditions which may lead to blindness), or damage to the enteric nerves and neural structures which relay messages from the intestine to the SCN (conditions which may lead to neurodegenerative disorders) can cause permanent dysfunction of the circadian rhythm and abnormal sleep behavior.

Dysfunction of the circadian rhythm manifests first and foremost by abnormal sleep patterns. Such abnormalities typically are mild at onset and worsen progressively over time. A common symptom of sleep disorder is a delay in the onset of sleep. This delay can be as long as several hours, and the individual may not be able to fall asleep until the early hours of the morning. Another common symptom is sleep fragmentation, meaning that the individual awakens several times during the course of the night. Once awakened, the individual may not be able to get back to sleep, and each awake fragment may last an hour or more, further reducing “total sleep time,” which is calculated by subtracting total time of the awake fragments from total time spent in bed. Total sleep time also diminishes with age, from about 14 to about 16 hours a day in newborns, to about 12 hours by one year of age, to about 7 to about 8 hours in young adults, progressively declining to about 5 to about 6 hours in elderly individuals. Total sleep time can be used to calculate an individual's “sleep age” and to compare it to their chronologic age. Significant discrepancies between sleep age and chronologic age are a reflection of the severity of the sleep disorder. “Sleep efficiency,” defined as the percentage of the time spent in bed asleep is another index that can be used to determine the severity of the sleep disorder. Sleep efficiency is said to be abnormal when the percentage is below about 70%.

Sleep disorders and/or sleep disturbances include but are not limited to REM-behavior disorders, disturbances in the Circadian rhythm, delayed sleep onset, sleep fragmentation, and hallucinations. Other sleep disorders or disturbances that can be treated and/or prevented according to the disclosed methods include but are not limited to hypersomnia (i.e., daytime sleepiness), parasomnias (such as nightmares, night terrors, sleepwalking, and confusional arousals), periodic limb movement disorders (such as Restless Leg Syndrome), jet lag, narcolepsy, advanced sleep phase disorder, non-24 hour sleep-wake syndrome.

Individuals with severe sleep disorders also typically suffer from day-time sleepiness. This can manifest as day-time “napping” for an hour or two, to “dosing off” for a few minutes during a film or to “micro-sleep” episodes lasting seconds to minutes, and of which the individual may or may not be aware. Narcolepsy is a rare and extreme form of day-time sleepiness, with the sudden onset of sleep causing the individual to fall down. Another form of sleep disturbance involves periods of loud snoring alternating with periods of “sleep apnea” (arrested breathing), a condition known as “sleep-disordered breathing.” “REM-behavior disorder” (RBD) or “REM-disturbed sleep”, is yet another sleep disturbance which occurs as a result of dysfunctional neural communication between the enteric nervous system, structures responsible for sleep in the brain stem and the SCN. In individuals with RBD, neural signaling which causes the paralysis (atonia) of muscles under voluntary control is impaired or altogether absent. As a consequence, “acting-out” of dreams occurs. This can range at one end of the spectrum from an increase in muscle tone detectable by electromyography (EMG) and accompanied by small movements of the hands and feet during REM sleep, to violent thrashing of arms and legs, kicking or punching a bed partner, speaking out loud or screaming, at the other end of the spectrum. Episodes of RBD can occur several times a night or very infrequently, once every few months. They can also be clustered, several occurring within a week, followed by periods of normal sleep. Unless the condition can be treated with a medication that restores normal functioning of the circadian rhythm and improves sleep patterns, individuals with RBD progress to neurodegenerative disorders.

Sleep disturbances include but are not limited to RBD, circadian rhythm dysfunction, delayed sleep onset, Restless leg syndrome, daytime sleepiness, and sleep fragmentation.

Sleep is increasingly recognized as important to public health, with sleep insufficiency linked to motor vehicle crashes, industrial disasters, and medical and other occupational errors. Unintentionally falling asleep, nodding off while driving, and having difficulty performing daily tasks because of sleepiness all may contribute to these hazardous outcomes. Persons experiencing sleep insufficiency are also more likely to suffer from chronic diseases such as hypertension, diabetes, depression, and obesity, as well as from cancer, increased mortality, and reduced quality of life and productivity. Sleep insufficiency may be caused by broad scale societal factors such as round-the-clock access to technology and work schedules, but sleep disorders such as insomnia or obstructive sleep apnea also play an important role. An estimated 50-70 million US adults have a sleep or wakefulness disorder.

A “normal” or “restful” sleep period is defined as a sleep period uninterrupted by wakefulness. Alternatively, a said period can be defined by the recommended or appropriate amount of sleep for the subject's age category, e.g., (i) infants 0-3 months=about 11 to about 19 hours; (ii) infants about 4 to about 11 months=about 12 to about 18 hours; (iii) toddlers about 1 to about 2 years=about 9 to about 16 hours; (iv) preschoolers about 3 to about 5 years=about 10 to about 14 hours; (v) school-aged children about 6 to about 13 years=about 7 to about 12 hours; (v) teenagers about 14 to about 17 years=about 7 to about 11 hours; (vi) young adults about 18 to about 25 years=about 6 to about 11 hours; (vii) adults about 26 to about 64 years=about 6 to about 10 hours; and (viii) older adults ≥65 years=about 5 to about 9 hours. Thus, for treating sleep disturbance in a subject, the treatment can result in a restful sleep period of at least about 4, about 5, about 6, about 7, about 8, about 9, about 10, about 11, or about 12 hours.

How much sleep is needed by a subject varies between individuals but generally changes with age. The National Institutes of Health suggests that school-age children need at least 10 hours of sleep daily, teens need 9-10 hours, and adults need 7-8 hours. According to data from the National Health Interview Survey, nearly 30% of adults reported an average of ≤6 hours of sleep per day in 2005-2007. Further, in 2009, only 31% of high school students reported getting at least 8 hours of sleep on an average school night Similar recommendations are provided by the National Sleep Foundation (https://sleepfoundation.org/press-release/national-sleep-foundation-recommends-new-sleep-times/page/0/1):

TABLE 1

| | | | May be | | |
|---|---|---|---|---|---|
| Age | Recommended | | appropriate | | Not recommended |
| Newborns | 14 to 17 | hours | 11 to 13 | hours | Less than 11 hours |
| 0-3 months | | | 18 to 19 | hours | More than 19 hours |
| Infants | 12 to 15 | hours | 10 to 11 | hours | Less than 10 hours |
| 4-11 months | | | 16 to 18 | hours | More than 18 hours |
| Toddlers | 11 to 14 | hours | 9 to 10 | hours | Less than 9 hours |
| 1-2 years | | | 15 to 16 | hours | More than 16 hours |
| Preschoolers | 10 to 13 | hours | 8 to 9 | hours | Less than 8 hours |
| 3-5 years | | | 14 | hours | More than 14 hours |
| School-aged | 9 to 11 | hours | 7 to 8 | hours | Less than 7 hours |
| Children | | | 12 | hours | More than 12 hours |
| 6-13 years | | | | | |
| Teenagers | 8 to 10 | hours | 7 | hours | Less than 7 hours |
| 14-17 years | | | 11 | hours | More than 11 hours |
| Young Adults | 7 to 9 | hours | 6 | hours | Less than 6 hours |
| 18-25 years | | | 10 to 11 | hours | More than 11 hours |
| Adults | 7 to 9 | hours | 6 | hours | Less than 6 hours |
| 26-64 years | | | 10 | hours | More than 10 hours |
| Older Adults | 7 to 8 | hours | 5 to 6 | hours | Less than 5 hours |
| ≥65 years | | | 9 | hours | More than 9 hours |

There are several different scientifically acceptable ways to measure a sleep period uninterrupted by wakefulness. First, electrodes attached to the head of a subject can measure electrical activity in the brain by electroencephalography (EEG). This measure is used because the EEG signals associated with being awake are different from those found during sleep. Second, muscle activity can be measured using electromyography (EMG), because muscle tone also differs between wakefulness and sleep. Third, eye movements during sleep can be measured using electro-oculography (EOG). This is a very specific measurement that helps to identify Rapid Eye Movement or REM sleep. Any of these methods, or a combination thereof, can be used to determine if a subject obtains a restful sleep period following administration of at least one aminosterol or a salt or derivative thereof to the subject.

Further, circadian rhythm regulation can be monitored in a variety of ways, including but not limited to monitoring wrist skin temperature as described by Sarabia et al. 2008. Similarly symptoms of RBD can be monitored using a daily diary and RBD questionnaire (Stiasny-Kolster et al. 2007).

In some embodiments, administration of a therapeutically effective fixed dose of an aminosterol composition to a hallucination patient with disturbed sleep results in improvement in frequency of normal or restful sleep as determined by a clinically recognized assessment scale for one or more types of sleep dysregulation, by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. The improvement can be measured using any clinically recognized tool or assessment.

Example 4 describes several tools used to measure and evaluate the effect of aminosterol treatment on sleep, including for example:

(1) Sleep Diary (participants completed a sleep diary on a daily basis throughout the study. The diaries included time into bed and estimated time to sleep as well as wake time and duration during the night);

(2) I-Button Temperature Assessment. The I-Button is a small, rugged self-sufficient system that measures temperature and records the results in a protected memory section. The Thermochron I-Button DS1921H (Maxim Integrated, Dallas, Tex.) was used for skin temperature measurement. I-Buttons were programmed to sample every 10 mins., and attached to a double-sided cotton sport wrist band using Velcro, with the sensor face of the I-Button placed over the inside of the wrist, on the radial artery of the dominant hand. Subjects removed and replaced the data logger when necessary (i.e., to have a bath or shower). The value of skin temperature assessment in sleep research is that the endogenous skin warming resulting from increased skin blood flow is functionally linked to sleep propensity. From the collected data, the mesor, amplitude, acrophase (time of peak temperature), Rayleight test (an index of interdaily stability), mean waveforms are calculated);

(3) Unified Parkinson's Disease Rating Scale (UPDRS), sections 1.7 (sleep problems), 1.8 (daytime sleepiness) and 1.13 (fatigue);

(4) Parkinson's Disease Fatigue Scale (PFS-16);

(5) REM Sleep Behavior Disorder Screening Questionnaire; and

(6) Parkinson's Disease Sleep Scale.

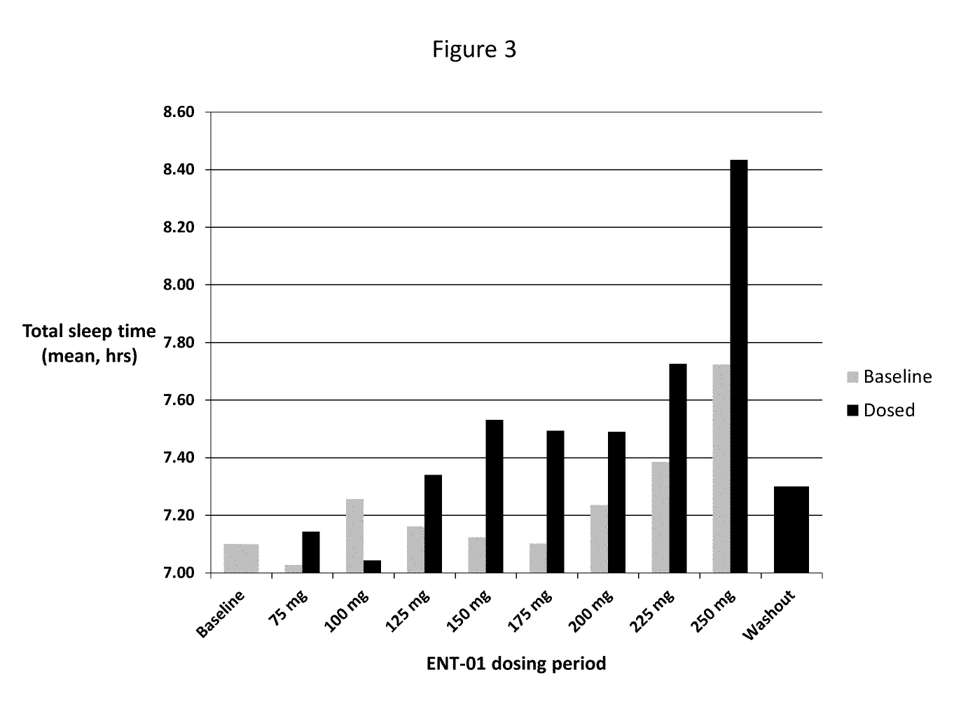

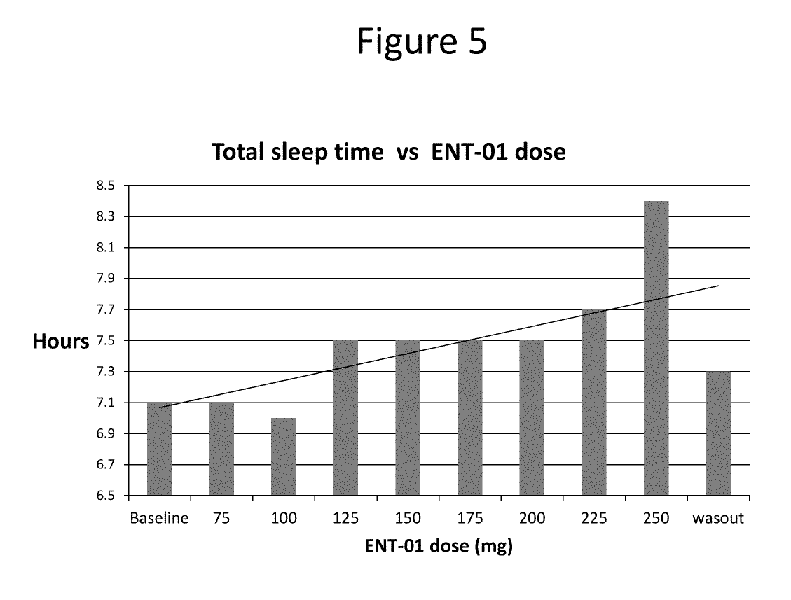

The data detailed in Example 4 described how circadian system status was evaluated by continuously monitoring wrist skin temperature (Thermochron iButton DS1921H; Maxim, Dallas) following published procedures (Sarabia et al. 2008). Further, an analysis was done with respect to the sleep data, the body temperature data, and fatigue data. The frequency of arm or leg thrashing reported in the sleep diary diminished progressively from 2.2 episodes/week at baseline to 0 at maximal dose (100% improvement). Total sleep time increased progressively from 7.1 hours at baseline to 8.4 hours at 250 mg (an 18% increase) and was consistently higher than baseline beyond 125 mg (FIGS. 3-5). Unlike stool-related indices, the improvement in many CNS symptoms persisted during wash-out.

Circadian rhythm of skin temperature was evaluable in 12 patients (i.e., those who had recordings that extended from baseline through washout). Circadian system functionality was evaluated by continuously monitoring wrist skin temperature using a temperature sensor (Thermochron iButton DS1921H; Maxim, Dallas, Tex.) (Sarabia et al. 2008). Briefly, this analysis includes the following parameters: (i) the inter-daily stability (the constancy of 24-hour rhythmic pattern over days, IS); (ii) intra-daily variability (rhythm fragmentation, IV); (iii) average of 10-minute intervals for the 10 hours with the minimum temperature (L10); (iv) average of 10-minute intervals for the 5 hours with the maximum temperature (M5) and the relative amplitude (RA), which was determined by the difference between M5 and L10, divided by the sum of both. Finally, the Circadian Function Index (CFI) was calculated by integrating IS, IV, and RA. Consequently, CFI is a global measure that oscillates between 0 for the absence of circadian rhythmicity and 1 for a robust circadian rhythm.

A comparison was performed of circadian rhythm parameters during the baseline, fixed dose and washout periods. Amino sterol administration improved all markers of healthy circadian function, including increasing rhythm stability, relative amplitude, and circadian function index, while reducing rhythm fragmentation. The improvement persisted for several of these circadian parameters during the wash-out period. (FIG. 6). Improvements were also seen in REM-behavior disorder (RBD) and sleep. RBD and total sleep time also improved progressively in a dose-dependent manner.

ii. Cognitive Impairment

Another symptom associated with hallucinations is cognitive impairment. Cognitive impairment, including mild cognitive impairment (MCI), is characterized by increased memory or thinking problems exhibited by a subject as compared to a normal subject of the same age. Approximately 15 to 20 percent of people age 65 or older have MCI, and MCI is especially linked to neurodegenerative conditions or synucleopathies like Parkinson's disease (PD). In 2002, an estimated 5.4 million people (22.%) in the United States over age 70 had cognitive impairment without dementia. Plassman et al. 2009.

Cognitive impairment may entail memory problems including a slight but noticeable and measurable decline in cognitive abilities, including memory and thinking skills. When MCI primarily affects memory, it is known as “amnestic MCI.” A person with amnestic MCI may forget information that would previously have been easily recalled, such as appointments, conversations, or recent events, for example. When MCI primarily affects thinking skills other than memory, it is known as “nonamnestic MCI.” A person with nonamnestic MCI may have a reduced ability to make sound decisions, judge the time or sequence of steps needed to complete a complex task, or with visual perception, for example.

Mild cognitive impairment is a clinical diagnosis. A combination of cognitive testing and information from a person in frequent contact with the subject is used to fully assess cognitive impairment. A medical workup includes one or more of an assessment by a physician of a subject's medical history (including current symptoms, previous illnesses, and family history), assessment of independent function and daily activities, assessment of mental status using brief tests to evaluate memory, planning, judgment, ability to understand visual information, and other key thinking skills, neurological examination to assess nerve and reflex function, movement, coordination, balance, and senses, evaluation of mood, brain imaging, or neuropsychological testing. Diagnostic guidelines for MCI have been developed by various groups, including the Alzheimer's Association partnered with the National Institute on Aging (NIA), an agency of the U.S. National Institutes of Health (NIH). Jack et al. 2011; McKhann et al. 2011; Albert et al. 2011. Recommendations for screening for cognitive impairment have been issued by the U.S. Preventive Services Task Force. Screening for Cognitive Impairment in Older Adults, U.S. Preventive Services Task Force (March 2014), https://www.uspreventiveservicestaskforce.org/Home/GetFileByID/1882. For example, the Mini Mental State Examination (MMSE) may be used. Palsetia et al., 2018; Kirkevold, 0. & Selbaek, G., 2015. With the MMSE, a score of 24 or greater (out of 30) may indicate normal cognition, with lower scores indicating severe (less than or equal to 9 points), moderate (10-18 points), or mild (19-23 points) cognitive impairment. Other screening tools include the Informant Questionnaire on Cognitive Decline in the Elderly (IQCODE), in which an average score of 3 indicates no cognitive decline and a score greater than 3 indicates some decline. Jorm, A. F., 2004. Alternatively, the 7-Minute Screener, Abbreviated Mental Test Score (AMTS), Cambridge Cognitive Examination (CAMCOG), Clock Drawing Test (CDT), General Practitioner Assessment of Cognition (GPCOG), Mini-Cog, Memory Impairment Screen (MIS), Montreal Cognitive Assessment (MoCA), Rowland Universal Dementia Assessment (RUDA), Self-Administered Gerocognitive Examination (SAGE), Short and Sweet Screening Instrument (SAS-SI), Short Blessed Test (SBT), St. Louis Mental Status (SLUMS), Short Portable Mental Status Questionnaire (SPMSQ), Short Test of Mental Status (STMS), or Time and Change Test (T&C), among others, are frequently employed in clinical and research settings. Cordell et al. 2013. Numerous examinations may be used, as no single tool is recognized as the “gold standard,” and improvements in score on any standardized examination indicate successful treatment of cognitive impairment, whereas obtaining a score comparable to the non-impaired population indicates total recovery.

In some embodiments, administration of a therapeutically effective fixed dose of an aminosterol composition to a hallucination patient in need results in improvement of cognitive impairment as determined by a clinically recognized assessment scale, by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. The improvement can be measured using any clinically recognized tool or assessment.

As detailed in Example 4, cognitive impairment and the improvement following aminosterol treatment were assessed using several tools:

(1) Mini Mental State Examination (MMSE);

(2) Trail Making Test (TMT) Parts A and B; and

(3) Unified Parkinson's Disease Rating Scale (UPDRS), sections 1.1 (cognitive impairment).

Assessments were made at baseline and at the end of the fixed dose and washout periods for Example 4, and an analysis was done with respect to the cognition symptoms. The results showed that the total UPDRS score was 64.4 at baseline, 60.6 at the end of the fixed dose period and 55.7 at the end of the wash-out period (a 13.5% improvement). Part 1 of the UPDRS (which includes section 1.1, cognitive impairment) had a mean baseline score of 11.6, a fixed aminosterol dose mean score of 10.6, and a wash-out mean score of 9.5, demonstrating an almost 20% improvement (UPDRS cognitive impairment is rated from 1=slight improvement to 4=severe impairment, so lower scores correlate with better cognitive function). In addition, MMSE improved from 28.4 at baseline to 28.7 during treatment and to 29.3 during wash-out (the MMSE has a total possible score of 30, with higher scores correlating with better cognitive function). Unlike stool-related indices, the improvement in many CNS symptoms persisted during wash-out.

3. Cerebral and General Ischemic Disorders

The methods and compositions of the invention may also be useful in treating, preventing, and/or delaying the onset or progression of hallucinations and/or a hallucination-related symptom, where the hallucination is correlated with abnormal α-S pathology, and/or correlated with dysfunctional DA neurotransmission, also known as dopaminergic dysfunction, and wherein the hallucination is also correlated with a cerebral or general ischemic disorder.

In some embodiments, the cerebral ischemic disorder comprises cerebral microangiopathy, intrapartal cerebral ischemia, cerebral ischemia during/after cardiac arrest or resuscitation, cerebral ischemia due to intraoperative problems, cerebral ischemia during carotid surgery, chronic cerebral ischemia due to stenosis of blood-supplying arteries to the brain, sinus thrombosis or thrombosis of cerebral veins, cerebral vessel malformations, or diabetic retinopathy.

In some embodiments, the general ischemic disorder comprises high blood pressure, high cholesterol, myocardial infarction, cardiac insufficiency, cardiac failure, congestive heart failure, myocarditis, pericarditis, perimyocarditis, coronary heart disease, angina pectoris, congenital heart disease, shock, ischemia of extremities, stenosis of renal arteries, diabetic retinopathy, thrombosis associated with malaria, artificial heart valves, anemias, hypersplenic syndrome, emphysema, lung fibrosis, or pulmonary edema.

### V. Combination Therapy

The methods of the invention can further comprise administering the amino sterol in combination with at least one additional active agent to achieve either an additive or synergistic effect. Such an additional agent can be administered via a method selected from the group consisting of concomitantly, as an admixture, separately and simultaneously or concurrently, and separately and sequentially.

Thus, the aminosterol compositions may be administered alone or in combination with other therapeutic agents. As noted above, the methods are useful in treating, preventing and/or slowing the onset or progression of the conditions described herein, including but not limited to hallucinations related Parkinson's disease, Alzheimer's disease, Huntington's Disease, schizophrenia, multiple sclerosis, and degenerative processes associated with aging. Thus, any active agent known to be useful in treating these conditions can be used in the disclosed, and either combined with the aminosterol compositions used in the methods, or administered separately or sequentially.

For example, in disclosed methods of treating, preventing and/or slowing the onset or progression of hallucinations, the aminosterol composition can be co-administered or combined with drugs commonly prescribed to treat the psychiatric disorders, neurological disorders, and neurodegenerative disorders described herein.

When combining more than one therapeutic compound for administering according to the disclosed methods, combinations may be administered either concomitantly, e.g., as an admixture; separately but simultaneously or concurrently; or sequentially. This includes presentations in which the combined agents are administered together as a therapeutic mixture, and also procedures in which the combined agents are administered separately but simultaneously, e.g., in separate pills/tablets into the same individual. Administration “in combination” further includes the separate administration of one of the compounds or agents given first, followed by the second.

In some embodiments, the additional active agent is a different amino sterol from that administered already administered to the subject. In some embodiments, a first aminosterol which is amino sterol 1436 or a salt or derivative thereof administered intranasally and a second aminosterol which is squalamine or a salt or derivative thereof administered orally.

In some embodiments, the additional active agent is an active agent used to treat hallucinations or a symptom thereof. In some embodiments, the active agent is selected from the group consisting of first-generation antipsychotics such as chlorpromazine (Thorazine®), fluphenazine (Prolixin®), haloperidol (Haldol®), perphenazine (Trilafon®), thioridazine (Mellaril®), thiothixene (Navane®), and trifluoperazine (Stelazine®); atypical antipsychotics such as aripiprazole (Abilify®), aripiprazole lauroxil (Aristada®), asenapine (Saphris®), clozapine (Clozaril®), iloperidone (Fanapt®), lurasidone (Latuda®), olanzapine (Zyprexa®), paliperidone (Invega Sustenna®), paliperidone palmitate (Invega Trinza®), quetiapine (Seroquel®), risperidone (Risperdal®), pimavanserin and ziprasidone (Geodon®).

For example, in methods of treating, preventing, and/or delaying the onset or progression of hallucinations or related symptoms associated with PD, the aminosterol composition can be co-administered or combined with drugs commonly prescribed to treat PD or related symptoms, such as levodopa (usually combined with a dopa decarboxylase inhibitor or COMT inhibitor), dopamine agonists and MAO-B inhibitors. Exemplary dopa decarboxylase inhibitors are carbidopa and benserazide. Exemplary COMT inhibitors are tolcapone and entacapone. Dopamine agonists include, for example, bromocriptine, pergolide, pramipexole, ropinirole, piribedil, cabergoline, apomorphine, lisuride, and rotigotine. MAO-B inhibitors include, for example, selegiline and rasagiline. Other drugs commonly used to treat PD include, for example, amantadine, anticholinergics, clozapine for psychosis, cholinesterase inhibitors for dementia, and modafinil for daytime sleepiness.

In methods of treating, preventing, and/or delaying the onset or progression of hallucinations or related symptoms associated with AD, the amino sterol composition can be co-administered or combined with drugs commonly prescribed to treat AD or related symptoms, such as glutamate, antipsychotic drugs, huperzine A, acetylcholinesterase inhibitors and NMDA receptor antagonists such as memantine (Akatinol®, Axura®, Ebixa®/Abixa®, Memox® and Namenda®). Examples of acetylcholinesterase inhibitors are donepezil (Aricept®), galantamine (Razadyne®), and rivastigmine (Exelon®).

In methods of treating, preventing, and/or delaying the onset or progression of hallucinations or related symptoms associated with diabetes and/or diabetes mellitus, including both Type 1 and Type 2 diabetes, or neuropathy of diabetes, the aminosterol composition can be co-administered or combined with drugs commonly prescribed to treat diabetes mellitus or related symptoms, such as insulin (NPH insulin or synthetic insulin analogs) (e.g., Humulin®, Novolin®) and oral antihyperglycemic drugs. Oral antihyperglycemic drugs include but are not limited to (1) biguanides such as metformin (Glucophage®); (2) Sulfonylureas such as acetohexamide, chlorpropamide (Diabinese®), glimepiride (Amaryl®), Glipizide (Glucotrol®), Tolazamide, Tolbutamide, and glyburide (Diabeta®, Micronase®); (3) Meglitinides such as repaglinide (Prandin®) and nateglinide (Starlix®); (4) Thiazolidinediones such as rosiglitazone (Avandia®) and pioglitazone (Actos®); (5) Alpha-glucosidase inhibitors such as acarbose (Precose®) and miglitol (Glyset®); (6) Dipeptidyl peptidase-4 inhibitors such as Sitagliptin (Januvia®); (7) Glucagon-like peptide agonists such as exenatide (Byetta®); and (8) Amylin analogs such as pramlintide (Symlin®).

In methods of treating, preventing, and/or delaying the onset or progression of hallucinations or related symptoms associated with Huntington's chorea or disease, the aminosterol composition can be co-administered or combined with drugs commonly prescribed to treat Huntington's chorea or related symptoms, such as medications prescribed to help control emotional and movement problems associated with Huntington's chorea. Such medications include, but are not limited to, (1) antipsychotic drugs, such as haloperidol and clonazepam; (2) drugs used to treat dystonia, such as acetylcholine regulating drugs (trihexyphenidyl, benztropine (Cogentin®), and procyclidine HCl); GABA-regulating drugs (diazepam (Valium®), lorazepam (Ativan®), clonazepam (Klonopin®), and baclofen (Lioresal®)); dopamine-regulators (levodopa/carbidopa (Sinemet®), bromocriptine (parlodel), reserpine, tetrabenazine); anticonvulsants (carbamazepine (Tegretol®) and botulinum toxin (Botox®)); and (3) drugs used to treat depression (fluoxetine, sertraline, and nortriptyline). Other drugs commonly used to treat HD include amantadine, tetrabenazine, dopamine blockers, and co-enzyme Qio.

In methods of treating, preventing, and/or delaying the onset or progression of hallucinations or related symptoms associated with peripheral sensory neuropathy, the aminosterol composition can be co-administered or combined with drugs commonly prescribed to treat peripheral sensory neuropathy or related symptoms. Peripheral sensory neuropathy refers to damage to nerves of the peripheral nervous system, which may be caused either by diseases of or trauma to the nerve or the side-effects of systemic illness. Drugs commonly used to treat this condition include, but are not limited to, neurotrophin-3, tricyclic antidepressants (e.g., amitriptyline), antiepileptic therapies (e.g., gabapentin or sodium valproate), synthetic cannabinoids (Nabilone) and inhaled cannabis, opiate and opioid derivatives, and pregabalin (Lyrica®).

In methods of treating, preventing, and/or delaying the onset or progression of hallucinations or related symptoms associated with traumatic head and/or spine injury, the aminosterol composition can be co-administered or combined with drugs commonly prescribed to treat traumatic head and/or spine injury or related symptoms, such as analgesics (acetaminophen, NSAIDs, salicylates, and opioid drugs such as morphine and opium) and paralytics.

In methods of treating, preventing, and/or delaying the onset or progression of hallucinations or related symptoms associated with stroke, the amino sterol composition can be co-administered or combined with drugs commonly prescribed to treat stroke or related symptoms, such as aspirin, clopidogrel, dipyridamole, tissue plasminogen activator (tPA), and anticoagulants (e.g., alteplase, warfarin, dabigatran).

In methods of treating, preventing, and/or delaying the onset or progression of hallucinations or related symptoms associated with ALS, the aminosterol composition can be co-administered or combined with drugs commonly prescribed to treat Amyotrophic lateral sclerosis or related symptoms, such as riluzole (Rilutek®), KNS-760704 (an enantiomer of pramipexole), olesoxime (TR019622), talampanel, arimoclomol, medications to help reduce fatigue, ease muscle cramps, control spasticity, reduce excess saliva and phlegm, control pain, depression, sleep disturbances, dysphagia, and constipation.

In methods of treating, preventing, and/or delaying the onset or progression of hallucinations or related symptoms associated with multiple sclerosis, the aminosterol composition can be co-administered or combined with drugs commonly prescribed to treat multiple sclerosis or related symptoms, such as corticosteroids (e.g., methylprednisolone), plasmapheresis, fingolimod (Gilenya®), interferon beta-1a (Avonex®, CinnoVex®, ReciGen® and Rebif®), interferon beta-1b (Betaseron® and Betaferon®), glatiramer acetate (Copaxone®), mitoxantrone, natalizumab (Tysabri®), alemtuzumab (Campath®), daclizumab (Zenapax®), rituximab, dirucotide, BHT-3009, cladribine, dimethyl fumarate, estriol, fingolimod, laquinimod, minocycline, statins, temsirolimus teriflunomide, naltrexone, and vitamin D analogs.

In methods of treating, preventing, and/or delaying the onset or progression of hallucinations or related symptoms associated with cerebral palsy, the aminosterol composition can be co-administered or combined with drugs commonly prescribed to treat cerebral palsy or related symptoms, such as botulinum toxin A injections.

In methods of treating, preventing, and/or delaying the onset or progression of hallucinations or related symptoms associated with epilepsy, the aminosterol composition can be co-administered or combined with drugs commonly prescribed to treat epilepsy or related symptoms, such as anticonvulsants (e.g., carbamazepine (Tegretol®), clorazepate (Tranxene®), clonazepam (Klonopin®), ethosuximide (Zarontin®), felbamate (Felbatol®), fosphenytoin (Cerebyx®), gabapentin (Neurontin®), lacosamide (Vimpat®), lamotrigine (Lamictal®), levetiracetam (Keppra®), oxcarbazepine (Trileptal®), phenobarbital (Luminal®), phenytoin (Dilantin®), pregabalin (Lyrica®), primidone (Mysoline®), tiagabine (Gabitril®), topiramate (Topamax®), valproate semisodium (Depakote®), valproic acid (Depakene®), and zonisamide (Zonegran®), clobazam (Frisium®), vigabatrin (Sabrir), retigabine, brivaracetam, seletracetam, diazepam (Valium® and Diastat®), lorazepam (Ativan®), paraldehyde (Paral®), midazolam (Versed®), pentobarbital (Nembutal®), acetazolamide (Diamox®), progesterone, adrenocorticotropic hormone (ACTH and Acthar®), various corticotropic steroid hormones (prednisone), and bromide.

In methods of treating, preventing, and/or delaying the onset or progression of hallucinations or related symptoms associated with cognitive impairment, the aminosterol composition can be co-administered or combined with drugs commonly prescribed to treat cognitive impairment, such as donepezil (Aricept®), galantamine (Razadyne®), and rivastigmine (Exelon®); and stimulants such as caffeine, amphetamine (Adderall®), lisdexamfetamine (Vyvanse®), and methylphenidate (Ritalin®); NMDA antagonists such as memantine (Nameda®); supplements such as ginko biloba, L-theanine, piracetam, oxiracitam, aniracetam, tolcapone, atomoxetine, ginseng, and Salvia officinalis.

In the methods of treating, preventing, and/or delaying the onset or progression of hallucinations or related symptoms associated with malignancies, the aminosterol composition can be co-administered or combined with drugs commonly used to treat malignancies. These include all known cancer drugs, such as but not limited to those listed at http://www.cancer.gov/cancertopics/druginfo/alphalist as of May 5, 2014, which is specifically incorporated by reference. In one embodiment, the drug commonly used to treat malignancies may be selected from the group consisting of actinomycin-D, alkeran, ara-C, anastrozole, BiCNU, bicalutamide, bleomycin, busulfan, capecitabine, carboplatin, carboplatinum, carmustine, CCNU, chlorambucil, cisplatin, cladribine, CPT-11, cyclophosphamide, cytarabine, cytosine arabinoside, cytoxan, dacarbazine, dactinomycin, daunorubicin, dexrazoxane, docetaxel, doxorubicin, DTIC, epirubicin, ethyleneimine, etoposide, floxuridine, fludarabine, fluorouracil, flutamide, fotemustine, gemcitabine, hexamethylamine, hydroxyurea, idarubicin, ifosfamide, irinotecan, lomustine, mechlorethamine, melphalan, mercaptopurine, methotrexate, mitomycin, mitotane, mitoxantrone, oxaliplatin, paclitaxel, pamidronate, pentostatin, plicamycin, procarbazine, steroids, streptozocin, STI-571, tamoxifen, temozolomide, teniposide, tetrazine, thioguanine, thiotepa, tomudex, topotecan, treosulphan, trimetrexate, vinblastine, vincristine, vindesine, vinorelbine, VP-16, xeloda, asparaginase, AIN-457, bapineuzumab, belimumab, brentuximab, briakinumab, canakinumab, cetuximab, dalotuzumab, denosumab, epratuzumab, estafenatox, farletuzumab, figitumumab, galiximab, gemtuzumab, girentuximab (WX-G250), herceptin, ibritumomab, inotuzumab, ipilimumab, mepolizumab, muromonab-CD3, naptumomab, necitumumab, nimotuzumab, ocrelizumab, ofatumumab, otelixizumab, ozogamicin, pagibaximab, panitumumab, pertuzumab, ramucirumab, reslizumab, rituximab, REGN88, solanezumab, tanezumab, teplizumab, tiuxetan, tositumomab, trastuzumab, tremelimumab, vedolizumab, zalutumumab, zanolimumab, SFC, accutane hoffmann-1a roche, AEE788 novartis, AMG-102, anti neoplaston, AQ4N (Banoxantrone), AVANDIA (Rosiglitazone Maleate), avastin (Bevacizumab) genetech, BCNU, biCNU carmustine, CCI-779, CCNU, CCNU lomustine, celecoxib (Systemic), chloroquine, cilengitide (EMD 121974), CPT-11 (CAMPTOSAR, Irinotecan), dasatinib (BMS-354825, Sprycel), dendritic cell therapy, etoposide (Eposin, Etopophos, Vepesid), GDC-0449, gleevec (imatinib mesylate), gliadel wafer, hydroxychloroquine, IL-13, IMC-3G3, immune therapy, iressa (ZD-1839), lapatinib (GW572016), methotrexate for cancer (Systemic), novocure, OSI-774, PCV, RAD001 novartis (mTOR inhibitor), rapamycin (Rapamune, Sirolimus), RMP-7, RTA 744, simvastatin, sirolimus, sorafenib, SU-101, SU5416 sugen, sulfasalazine (Azulfidine), sutent (Pfizer), TARCEVA (erlotinib HCl), taxol, TEMODAR schering-plough, TGF-B anti-sense, thalomid (thalidomide), topotecan (Systemic), VEGF trap, VEGF-trap, vorinostat (SAHA), XL 765, XL184, XL765, zarnestra (tipifarnib), ZOCOR (simvastatin), cyclophosphamide (Cytoxan), (Alkeran), chlorambucil (Leukeran), thiopeta (Thioplex), busulfan (Myleran), procarbazine (Matulane), dacarbazine (DTIC), altretamine (Hexalen), clorambucil, cisplatin (Platinol), ifosafamide, methotrexate (MTX), 6-thiopurines (Mercaptopurine [6-MP], Thioguanine [6-TG]), mercaptopurine (Purinethol), fludarabine phosphate, (Leustatin), flurouracil (5-FU), cytarabine (ara-C), azacitidine, vinblastine (Velban), vincristine (Oncovin), podophyllotoxins (etoposide {VP-16} and teniposide {VM-26}), camptothecins (topotecan and irinotecan), taxanes such as paclitaxel (Taxol) and docetaxel (Taxotere), (Adriamycin, Rubex, Doxil), dactinomycin (Cosmegen), plicamycin (Mithramycin), mitomycin: (Mutamycin), bleomycin (Blenoxane), estrogen and androgen inhibitors (Tamoxifen), gonadotropin-releasing hormone agonists (Leuprolide and Goserelin (Zoladex)), aromatase inhibitors (Aminoglutethimide and Anastrozole (Arimidex)), amsacrine, asparaginase (El-spar), mitoxantrone (Novantrone), mitotane (Lysodren), retinoic acid derivatives, bone marrow growth factors (sargramostim and filgrastim), amifostine, pemetrexed, decitabine, iniparib, olaparib, veliparib, everolimus, vorinostat, entinostat (SNDX-275), mocetinostat (MGCD0103), panobinostat (LBH589), romidepsin, valproic acid, flavopiridol, olomoucine, roscovitine, kenpaullone, AG-024322 (Pfizer), fascaplysin, ryuvidine, purvalanol A, NU2058, BML-259, SU 9516, PD-0332991, P276-00, geldanamycin, tanespimycin, alvespimycin, radicicol, deguelin, BIIB021, cis-imidazoline, benzodiazepinedione, spiro-oxindoles, isoquinolinone, thiophene, 5-deazaflavin, tryptamine, aminopyridine, diaminopyrimidine, pyridoisoquinoline, pyrrolopyrazole, indolocarbazole, pyrrolopyrimidine, dianilinopyrimidine, benzamide, phthalazinone, tricyclic indole, benzimidazole, indazole, pyrrolocarbazole, isoindolinone, morpholinyl anthracycline, a maytansinoid, ducarmycin, auristatins, calicheamicins (DNA damaging agents), α-amanitin (RNA polymerase II inhibitor), centanamycin, pyrrolobenzodiazepine, streptonigtin, nitrogen mustards, nitrosorueas, alkane sulfonates, pyrimidine analogs, purine analogs, antimetabolites, folate analogs, anthracyclines, taxanes, vinca alkaloids, topoisomerase inhibitors, hormonal agents, and any comthe sbination thereof.

In the methods of treating, preventing, and/or delaying the onset or progression of hallucinations or related symptoms associated with depression, the aminosterol composition can be co-administered or combined with drugs commonly used to treat depression. These include selective serotonin reuptake inhibitors (SSRIs) such as citalopram (Celexa®, Cipramil®), escitalopram (Lexapro®, Cipralex®), paroxetine (Paxil®, Seroxat®), fluoxetine (Prozac®), fluvoxamine (Luvox®, Faverin®), sertraline (Zoloft®, Lustral®), indalpine (Upstene®), zimelidine (Normud®, Zelmid®); serotonin-norepinephrine reuptake inhibitors (SNRIs) such as desvenlafaxine (Pristiq®), duloxetine (Cymbalta®), levomilnacipran (Fetzima®), milnacipran (Ixel®, Savella®), venlafaxine (Effexor®); serotonin modulators and stimulators (SMSs) such as vilazodone (Viibryd®), vortioxetine (Trintellix®); serotonin antagonists and reuptake inhibitors such as nefazodone (Dutonin®, Nefadar®, Serzone®), trazodone (Desyrel®), etoperidone; norepinephrine reuptake inhibitors (NRIs) such as reboxetine (Edronax®), teniloxazine (Lucelan®, Metatone®), viloxazine (Vivalan®), atomoxetine (Strattera®); norepinephrine-dopamine reuptake inhibitors such as bupropion (Wellbutrin®), amineptine (Survector®, Maneon®), nomifensine (Merital®, Alival®), methylphenidate (Ritalin®, Concerta®), lisdexamfetamine (Vyvanse®); tricyclic antidepressants such asamitriptyline (Elavil®, Endep®), amitriptylinoxide (Amioxid®, Ambivalon®, Equilibrin®), clomipramine (Anafranil®), desipramine (Norpramin®, Pertofrane®), dibenzepin (Noveril®, Victoril®), dimetacrine (Istonil®), dosulepin (Prothiaden®), doxepin (Adapin®, Sinequan®), imipramine (Tofranil®), lofepramine (Lomont®, Gamanil®), melitracen (Dixeran®, Melixeran®, Trausabun®), nitroxazepine (Sintamil®), nortriptyline (Pamelor®, Aventyl®), noxiptiline (Agedal®, Elronon®, Nogedal®), opipramol (Insidon®), pipofezine (Azafen®/Azaphen®), protriptyline (Vivactil®), trimipramine (Surmontil®), butriptyline (Evadyne®), demexiptiline (Deparon®, Tinoran®), fluacizine (Phtorazisin®), imipraminoxide (Imiprex®, Elepsin®), iprindole (Prondol®, Galatur®, Tertran®), metapramine (Timaxel®), propizepine (Depressin®, Vagran®), quinupramine (Kinupril®, Kevopril®), tiazesim (Altinil®), tofenacin (Elamol®, Tofacine®), amineptine (Survector®, Maneon®), tianeptine (Stabion®, Coaxil®); tetracyclic antidepressants such as amoxapine (Asendin®), maprotiline (Ludiomil®), mianserin (Bolvidon®, Norval®, Tolvon®), mirtazapine (Remeron®), setiptiline (Tecipul®), mianserin, mirtazapine, setiptiline; monoamine oxidase inhibitors (MAOIs) such as isocarboxazid (Marplan®), phenelzine (Nardil®), tranylcypromine (Parnate®), benmoxin (Neuralex®), iproclozide (Sursum®), iproniazid (Marsilid®), mebanazine (Actomol®), nialamide (Niamid®), octamoxin (Ximaol®), pheniprazine (Catron®), phenoxypropazine (Drazine®), pivhydrazine (Tersavid®), safrazine (Safra®), selegiline (Eldepryl®, Zelapar®, Emsam®), caroxazone (Surodil®, Timostenil®), metralindole (Inkazan®), moclobemide (Aurorix®, Manerix®), pirlindole (Pirazidol®), toloxatone (Humoryl®), eprobemide (Befol®), minaprine (Brantur®, Cantor®), bifemelane (Alnert®, Celeport®); atypical antipsychotics such as amisulpride (Solian®), lurasidone (Latuda®), quetiapine (Seroquel®); and N-methyl D-aspartate (NMDA) antagonists such ketamine (Ketalar®).

### VI. Definitions

The following definitions are provided to facilitate understanding of certain terms used throughout this specification.

Technical and scientific terms used herein have the meanings commonly understood by one of ordinary skill in the art, unless otherwise defined. Any suitable materials and/or methodologies known to those of ordinary skill in the art can be utilized in carrying out the methods described herein.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

As used herein, “about” will be understood by persons of ordinary skill in the art and will vary to some extent depending upon the context in which it is used. Certain ranges are presented herein with numerical values being preceded by the term “about”. The term “about” is used herein to provide literal support for the exact number that it precedes, as well as a number that is near to or approximately the number that the term precedes. If there are uses of the term which are not clear to persons of ordinary skill in the art, given the context in which it is used, “about” will mean up to plus or minus 10% of the particular term, for example, ±1%, ±2%, ±3%, ±4%, ±5%, ±6%, ±7%, ±8%, ±9% or ±10%.

As used in the description of the invention and the appended claims, the singular forms “a”, “an” and “the” are used interchangeably and intended to include the plural forms as well and fall within each meaning, unless the context clearly indicates otherwise. Also, as used herein, “and/or” refers to and encompasses any and all possible combinations of one or more of the listed items, as well as the lack of combinations when interpreted in the alternative (“or”).

As used herein the term “aminosterol” encompasses squalamine or a derivative thereof, an isomer or prodrug of squalamine, Aminosterol 1436 or a derivative thereof, an isomer or prodrug of Aminosterol 1436, or a naturally occurring aminosterol isolated from Squalus acanthias or a derivative thereof, as described herein. “Amino sterols” useful in the invention also encompass a pharmaceutically equivalent salt of any aminosterol compound described herein. These compounds, and pharmaceutically acceptable salts thereof, are collectively referred to herein as “squalamine” and “aminosterols.” Thus, the term “aminosterol” as used herein is intended to encompass the broader class that includes both squalamine and the known naturally occurring amino sterols.

As used herein, the phrase “therapeutically effective amount” means a dose that provides the specific pharmacological effect for which the compound or compounds are being administered. It is emphasized that a therapeutically effective amount will not always be effective in achieving the intended effect in a given subject, even though such dose is deemed to be a therapeutically effective amount by those of skill in the art. For convenience only, exemplary dosages are provided herein. Those skilled in the art can adjust such amounts in accordance with standard practices as needed to treat a specific subject. The therapeutically effective amount may vary based on the route of administration and dosage form, the age and weight of the subject, and/or the severity of the subject's condition. For example one of skill in the art would understand that the therapeutically effective amount for treating a small individual may be different from the therapeutically effective amount for treating a large individual. In the context of treating hallucinations, the type of hallucination and any underlying pathophysiology that contributes to the hallucinations may have a bearing on the dose needed to therapeutically effective.

The terms “treatment” or “treating” as used herein includes preventing, reducing, ameliorating, or eliminating one or more symptoms or effects of the hallucinations being treating.

The term “administering” as used herein includes prescribing for administration as well as actually administering, and includes physically administering by the subject being treated or by another.

As used herein “subject” or “patient” or “individual” refers to any subject, patient, or individual, such as a subject suffering from hallucinations or at risk of suffering from hallucinations, and the terms are used interchangeably herein. In this regard, the terms “subject,” “patient,” and “individual” includes mammals, and, in particular humans.

As used herein each “defined period of time” may be independently selected from, for example, about 12 hours, about 24 hours, about 2 days, about 3 days, about 4 days, about 5 days, about 6 days, about one week, about 2, about 3, or about 4 weeks, about 1, about 2, about 3, about 4, about 5, about 6, about 7, about 8, about 9, about 10, about 11, or about 12 months, or about 1 year or longer.

The following examples are provided to illustrate the present invention. It should be understood, however, that the invention is not to be limited to the specific conditions or details described in these examples. Throughout the specification, any and all references to a publicly available document, including a U.S. patent, are specifically incorporated by reference.

### EXAMPLES

### Example 1

The purpose of this example was to evaluate the effectiveness of treating hallucinations in a Parkinson's disease patient with an amino sterol administered orally.

MV1, an 82-year-old man with a 13-year history of PD had been suffering from daily visual hallucinations for 5 years. MV1 reported that the hallucinations occurred at night. MV1 was aware that the apparitions were unreal, and he was fully awake when they occurred. The hallucinations consisted mostly of faceless dead relatives who came into his bedroom, sat on his bed, or in a chair, or walked around. The hallucinations were not threatening, and MV1 did not hallucinate that the apparitions talked to him. Sometimes MV1 would shout at the hallucinations to go away and the hallucinations would disappear. MV1 also had tactile hallucinations, causing MV1 to feel that insects such as cockroaches were climbing up his legs. He would bend down and attempt to brush them off his feet and legs. MV1 also had tactile hallucinations of the hands “as if someone was picking at them”. He was not treated with any antipsychotic medication and he didn't take any sleeping pills or sedatives. He also suffered from REM-behavior disorder (RBD), and had thrashing of arms and legs during his sleep. His wife had moved out of the bed several years prior because of the thrashing, screaming and hallucinations.

The patient was started on 75 mg squalamine daily. As the dose was increased, MV1 reported that he was hallucinating less frequently. He was also sleeping better. When the daily dose of squalamine was increased to 125 mg, the hallucinations disappeared completely, and his sleep and RBD continued to improve. Subsequently, the dose was increased to 175 mg, and maintained at 175 mg per day for another week or two, before discontinued. MV1 remained hallucination-free for another 30 days after discontinuation of the treatment.

This example demonstrates that an aminosterol such as squalamine can effectively treat hallucinations in PD subjects.

### Example 2

The purpose of this example was to evaluate the effectiveness of treating hallucinations in a Parkinson's disease patient with an amino sterol administered orally.

NY1, a 63-year-old man with a 5-year history of PD suffered from daily hallucinations. The hallucinations had been occurring for a number of years. The hallucinations occurred any time of day or night.

NY1 was started on squalamine 75 mg daily and then escalated to 100 mg daily. At 100 mg squalamine daily, NY1 noticed that his hallucinations were occurring less frequently, and he had hallucinations no more than once or twice a week. Upon increasing the dose to 125 mg per day, the hallucinations disappeared altogether. He was maintained at 125 mg per day for about a week, after which medication was discontinued. NY1 remained hallucination-free for 9 days following discontinuation.

This example demonstrates that an aminosterol such as squalamine can effectively treat hallucinations in PD subjects.

### Example 3

The purpose of this example was to evaluate the effectiveness of treating hallucinations in a Parkinson's disease patient with an amino sterol administered orally.

BC, an 80-year-old woman with a 10-year history of Parkinson's Disease suffered from frequent hallucinations. The hallucinations would occur at night and consist of people roaming around in her bedroom, such as a young lady sitting on her bed, or a priest standing by the bed. She was fully awake and aware that the visions were unreal. She also suffered from fragmented sleep and REM-behavior disorder (RBD).

BC was started on a squalamine dose of 75 mg daily, which was increased to a daily dose of 175 mg that was maintained for a 3-month period. During the 3 months, she had no hallucinations. Soon after discontinuing treatment, vivid hallucinations returned, occurring nightly. She described the hallucinations as cooks with white top hats and cleaners in blue uniforms. Squalamine was restarted at 125 mg daily and the hallucinations vanished. Upon discontinuing the medication, the hallucinations returned. This cycle of stopping and resuming squalamine treatment was repeated three times, and the hallucinations abated every time the squalamine treatment was resumed and hallucinations came back every time squalamine treatment was stopped. A portion of her sleep diary is shown below in Table 2.

TABLE 2
Sleep Diary for Patient BC

| Date | Hallucinations vs starting and stopping squalamine |
|---|---|
| 7.31 | 150 mg squalamine |
| 8.1 | STOP squalamine administration |
| 8.4 | Apparitions, cooks in tall hats |
| 8.5 | Apparitions, cleaning crew in blue uniforms |
| 8.6 | Apparitions, severely disturbed sleep |
| 8.7 | Apparitions |
| 8.8 | START 125 mg squalamine |
| 8.9 | No hallucinations |
| 9.9 | No hallucinations since last entry |
| 10.4 | No hallucinations since restarting |
| 10.5 | STOP squalamine administration |
| 10.15 | Apparitions back with a vengeance |

This example demonstrates that an aminosterol such as squalamine can effectively treat hallucinations in PD subjects.

### Example 4

This example describes an exemplary method of treating and/or preventing symptoms of Parkinson's disease (PD) in a clinical trial setting.

Overview:

The subjects of the trial all had PD and experienced constipation, which is a characteristic of PD. The primary objectives of the trial involving patients with PD and constipation were to evaluate the safety and pharmacokinetics of oral squalamine (ENT-01) and to identify the dose required to improve bowel function, which was used as a clinical endpoint.

Several non-constipation PD symptoms were also assessed as endpoints, including, for example, (1) sleep problems, including daytime sleepiness; (2) non-motor symptoms, such as (i) depression (including apathy, anxious mood, as well as depression), (ii) cognitive impairment (e.g., using trail making test and the UPDRS), (iii) hallucinations (e.g., using The University of Miami Parkinson's Disease Hallucinations Questionnaire (UM-PDHQ) and the UPDRS, (iv) dopamine dysregulation syndrome (UPDRS), (v) pain and other sensations, (vi) urinary problems, (vii) light headedness on standing, and (viii) fatigue (e.g., using Parkinson's Disease Fatigue Scale 9PFS-lt and the UPDRS); (3) motor aspects of experiences of daily living, such as (i) speech, (ii) saliva and drooling, (iii) chewing and swallowing, (iv) eating tasks, (v) dressing, (vi) hygiene, (vii) handwriting; (viii) doing hobbies and other activities, (ix) turning in bed, (x) tremor, (xi) getting out of bed, a car, or a deep chair, (xii) walking and balance, (xiii) freezing; (4) motor examination, such as (i) speech, (ii) facial expression, (iii) rigidity, (ix) finger tapping, (v) hand movements, (vi) pronation-supination movements of hands, (vii) toe tapping, (viii) leg agility, arising from chair, (ix) gait, (x) freezing of gait, (xi) postural stability, (xii) posture, (xiii) global spontaneity of movement (body bradykinesia), (xiv) postural tremor of the hands, (xv) kinetic tremor of the hands, (xvi) rest tremor amplitude, (xvii) constancy of rest tremor; (5) motor complications, such as (i) time spent with dyskinesias, (ii) functional impact of dyskinesias, (iii) time spent in the off state, (iv) functional impact of fluctuations, (v) complexity of motor fluctuations, and (vi) painful off-state dystonia.

Active Agent & Dosing:

Squalamine (ENT-01; Enterin, Inc.) was formulated for oral administration in the trial. The active ion of ENT-01, squalamine, an amino sterol originally isolated from the dogfish shark, has been shown to reverse gastrointestinal dysmotility in several mouse models of PD. In addition, ENT-01 has been shown to inhibit the formation of aggregates of αS both in vitro, and in a C. elegans model of PD in vivo (Perni et al. 2017). In the C. elegans model, squalamine produced a complete reversal of muscle paralysis.

ENT-01 is the phosphate salt of squalamine. For this study it has been formulated as a small 25 mg coated tablet. Dosing ranged from 25 mg to 250 mg, with dosages greater than 25 mg requiring multiple pills (e.g., 50 mg=two 25 mg pills). Dosing instructions=take 60 mins before breakfast with 8 oz. water. The dose was taken by each patient upon awakening on an empty stomach along with 8 oz. of water simultaneously to dopamine. The subject was not allowed to ingest any food for at least 60 minutes after study medication. The compound is highly charged and will adsorb to foodstuffs, so it was administered prior to feeding.

The phosphate salt of squalamine (ENT-01) is weakly soluble in water at neutral pH but readily dissolves at pH<3.5 (the pH of gastric fluid). Squalamine, as the highly water soluble dilactate salt has been extensively studied in over three Phase 1 and eight Phase 2 human clinical trials as an intravenous agent for the treatment of cancer and diabetic retinopathy. The compound is well tolerated in single and repeat intravenous administration, alone or in combination with other agents, to doses of at least 300 mg/m²).

In the current clinical trial, squalamine (ENT-01) was administered orally to subjects with PD who have long standing constipation. Although this trial was the first in man oral dosing study of ENT-01, humans have long been exposed to low doses of squalamine (milligram to microgram) in the various commercial dogfish shark liver extracts available as nutraceuticals (e.g., Squalamax). In addition, following systemic administration squalamine is cleared by the liver and excreted as the intact molecule (in mice) into the duodenum through the biliary tract. Drug related GI toxicology has not been reported in published clinical trials involving systemic administration of squalamine.

Squalamine (ENT-01) has limited bioavailability in rats and dogs. Based on measurement of portal blood concentrations following oral dosing of radioactive ENT-01 to rat's absorption of ENT-01 from the intestine is low. As a consequence, the principal focus of safety is on local effects on the gastrointestinal tract. However, squalamine (ENT-01) appears to be well tolerated in both rats and dogs.

The starting dose in the Stage 1 segment of the trial was 25 mg (0.33 mg/kg for a 75 kg subject). The maximum single dose in Stage 1 was 200 mg (2.7 mg/kg for a 75 kg subject). The maximum dose evaluated in Stage 2 of the trial was 250 mg/day (3.3 mg/kg/day for a 75 kg subject), and the total daily dosing exposure lasted no longer than 25 days.

The daily dosing range in the clinical trial was from 25 mg (14.7 mg/m²) to 250 mg (147 mg/m²). Oral dosing of squalamine (ENT-01), because of its low oral bioavailability, is not anticipated to reach significant plasma concentrations in human subjects. In preclinical studies, squalamine (ENT-01) exhibited an oral bioavailability of about 0.1% in both rats and dogs. In Stage 1 of this phase 2 study, oral dosing up to 200 mg (114 mg/m²) yielded an approximate oral bioavailability of about 0.1%, based on a comparison of a pharmacokinetic data of the oral dosing and the pharmacokinetic data measured during prior phase 1 studies of IV administration of squalamine.

Study Protocol:

The multicenter Phase 2 trial was conducted in two Stages: a dose-escalation toxicity study in Stage 1 and a dose range-seeking and proof of efficacy study in Stage 2.

PD symptoms were assessed using a number of different tools:

(1) Numeric Rating Scales for Pain and Swelling (scale of 0-10, with 0=no pain and 10=worst pain ever experienced);

(2) Rome-IV Criteria for Constipation (7 criteria, with constipation diagnosis requiring two or more of the following: (i) straining during at least 25% of defecations, (ii) lumpy or hard stools in at least 25% of defecations, (iii) sensation of incomplete evacuation for at least 25% of defecations, (iv) sensation of anorectal obstruction/blockage for at least 25% of defecations; (v) manual maneuvers to facilitate at least 25% of defecations; (vi) fewer than 3 defecations per week; and (vii) loose stools are rarely present without the use of laxatives;

(3) Constipation—Ease of Evacuation Scale (from 1-7, with 7=incontinent, 4=normal, and 1=manual disimpaction);

(4) Bristol Stool Chart, which is a patient-friendly means of categorizing stool characteristics (assessment of stool consistency is a validated surrogate of intestinal motility) and Stool Diary;

(5) Sleep Diary (participants completed a sleep diary on a daily basis throughout the study. The diaries included time into bed and estimated time to sleep as well as wake time and duration during the night);

(6) I-Button Temperature Assessment. The I-Button is a small, rugged self-sufficient system that measures temperature and records the results in a protected memory section. The Thermochron I-Button DS1921H (Maxim Integrated, Dallas, Tex.) was used for skin temperature measurement. I-Buttons were programmed to sample every 10 mins., and attached to a double-sided cotton sport wrist band using Velcro, with the sensor face of the I-Button placed over the inside of the wrist, on the radial artery of the dominant hand. Subjects removed and replaced the data logger when necessary (i.e., to have a bath or shower). The value of skin temperature assessment in sleep research is that the endogenous skin warming resulting from increased skin blood flow is functionally linked to sleep propensity. From the collected data, the mesor, amplitude, acrophase (time of peak temperature), Rayleight test (an index of interdaily stability), mean waveforms are calculated);

(7) Non-motor Symptoms Questionnaire (NMSQ);

(8) Beck Depression Inventory (BDI-II);

(9) Unified Parkinson's Disease Rating Scale (UPDRS), which consists of 42 items in four subscales (Part I=Non-Motor Aspects of Experiences of Daily Living (nM-EDL) (1.1 cognitive impairment, 1.2 hallucinations and psychosis, 1.3 depressed mood, Part II=Motor Aspects of Experiences of Daily Living (M-EDL), Part III=Motor Examination, and Part IV=Motor Complications;

(10) Mini Mental State Examination (MMSE);

(11) Trail Making Test (TMT) Parts A and B;

(12) The University of Miami Parkinson's Disease Hallucinations Questionnaire (UM-PDHQ);

(13) Parkinson's Disease Fatigue Scale (PFS-16);

(14) Patient Assessment of Constipation Symptoms (PAC-SYM);

(15) Patient Assessment of Constipation Quality of Life (PAC-QOL);

(16) REM Sleep Behavior Disorder Screening Questionnaire; and

(17) Parkinson's Disease Sleep Scale.

Exploratory end-points, in addition to constipation, included for example, (i) depression assessed using the Beck Depression Inventory (BDI-II) (Steer et al. 2000) and Unified Parkinson's Disease Rating Scale (UPDRS); (ii) cognition assessed using the Mini Mental State Examination (MMSE) (Palsteia et al. 2018), Unified Parkinson's Disease Rating Scale (UPDRS), and Trail Making Test (TMT); (iii) sleep and REM-behavior disorder (RBD) using a daily sleep diary, I-Button Temperature Assessment, a REM sleep behavior disorder (RBD) questionnaire (RBDQ) (Stiasny-Kolster et al. 2007), and the UPDRS; (iv) hallucinations assessed using the PD hallucinations questionnaire (PDHQ) (Papapetropoulos et al. 2008), the UPDRS, and direct questioning; (v) fatigue using the Parkinson's Disease Fatigue Scale (PFS-16) and the UPDRS; (vi) motor functions using the UPDRS; and (vii) non-motor functions using the UPDRS.

Assessments were made at baseline and at the end of the fixed dose and washout periods. Circadian system status was evaluated by continuously monitoring wrist skin temperature (Thermochron iButton DS1921H; Maxim, Dallas) following published procedures (Sarabia et al. 2008).

Based on these data, it is believed that administration of squalamine (ENT-01), a compound that can displace αS from membranes in vitro, reduces the formation of neurotoxic αS aggregates in vivo, and stimulates gastrointestinal motility in patients with PD and constipation. The observation that the dose required to achieve a prokinetic response increases with constipation severity supports the hypothesis that the greater the burden of αS impeding neuronal function, the higher the dose of squalamine (ENT-01) required to restore normal bowel function.

Study Design:

A multicenter Phase 2 trial was conducted in two Stages: a dose-escalation toxicity study in Stage 1 and a dose range-seeking and proof of efficacy study in Stage 2. The protocol was reviewed and approved by the institutional review board for each participating center and patients provided written informed consent.

Following successful screening, all subjects underwent a 14-day run-in period where the degree of constipation was assessed through a validated daily log (Zinsmeister et al. 2013) establishing baseline CSBMs/week. Subjects with an average of <3 CSBMs/week proceeded to dosing.

In Stage 1, ten (10) PD patients received a single escalating dose of squalamine (ENT-01) every 3-7 days beginning at 25 mg and continuing up to 200 mg or the limit of tolerability, followed by 2-weeks of wash-out. Duration of this part of the trial was 22-57 days. The 10 subjects in the sentinel group were assigned to Cohort 1 and participated in 8 single dosing periods. Tolerability limits included diarrhea or vomiting. A given dose was considered efficacious in stimulating bowel function (prokinetic) if the patient had a complete spontaneous bowel movement (CSBM) within 24 hours of dosing.

Each dose period was staggered, so that subjects 1-2 were administered a single dose of the drug at the lowest dose of 25 mg. Once 24 hours have elapsed, and provided there are no safety concerns, the patient was sent home and brought back on day 4-8 for the next dose. During the days the subjects are home, they completed the daily diaries and e-mailed them to the study coordinators. Subjects 3-10 were dosed after the first 2 subjects have been observed for 72 hours, i.e. on Day 4. Subjects 1-2 were also brought back on Day 4-8 and given a single dose of 50 mg. Once another 24 hours have elapsed and provided there are no safety concerns, the patients were all sent home and instructed to return on Day 7 for the next dosing level. This single dosing regimen was continued until each subject was given a single dose of 200 mg or has reached a dose limiting toxicity (DLT). DLT was the dose which induces repeated vomiting, diarrhea, abdominal pain or symptomatic postural hypotension within 24 hours of dosing.

In Stage 2, 34 patients were evaluated. First, 15 new PD patients were administered squalamine (ENT-01) daily, beginning at 75 mg, escalating every 3 days by 25 mg to a dose that had a clear prokinetic effect (CSBM within 24 hours of dosing on at least 2 of 3 days at a given dose), or the maximum dose of 175 mg or the tolerability limit. This dose was then maintained (“fixed dose”) for an additional 3-5 days. After the “fixed dose”, these patients were randomly assigned to either continued treatment at that dose or to a matching placebo, for an additional 4-6 days prior to a 2-week wash-out.

A second cohort of 19 patients received squalamine (ENT-01) escalating from 100 mg/day to a maximum of 250 mg/day without subsequent randomization to squalamine (ENT-01) or placebo. Criteria for dose selection and efficacy were identical to those used in the previous cohort.

Patient Population:

Patients were between 18 and 86 years of age and diagnosed with PD by a clinician trained in movement disorders following the UK Parkinson's Disease Society Brain Bank criteria (Fahn et al. 1987). Patients were required to have a history of constipation as defined by <3 CSBMs/week and satisfy the Rome IV criteria for functional constipation (Mearin et al. 2016) at screening, which requires 2 or more of the following: Straining during at least 25% of defecations; lumpy or hard stools in at least 25% of defecations; sensation of incomplete evacuation in at least 25% of defecations; sensation of anorectal obstruction/blockage in at least 25% of defecations; and/or manual maneuvers to facilitate at least 25% of defecations.

Baseline characteristics of patients are shown in Table 3. Patients in Stage 2 had somewhat longer duration of Parkinson's disease and higher UPDRS scores than participants in Stage 1.

TABLE 3
Baseline Characteristics of Dosed Patients

| | Stage 1** | Stage 2*** | |
|---|---|---|---|
| Characteristic | (n = 10) | (n = 34) | Total (n = 44) |
| Sex- no. (%) | | | |
| Male | 5 (50) | 25 (73.5) | 30 (68.1) |
| Female | 5 (50) | 9 (26.5) | 14 (31.8) |
| White race-no. (%) | 8 (80) | 34 (100) | 42 (95.54) |
| Age-yr | | | |
| Mean | 65.0 | 74.5 | 72.5 |
| Range | 58-70.5 | 60.6-84.2 | 58-84.2 |
| Age at PD diagnosis-yr | | | |
| Mean | 61.1 | 67.7 | 66.2 |
| Range | 54.2-69 | 50.6-82.5 | 50.6-82.5 |
| Duration of PD-yr | | | |
| Mean | 4.2 | 6.8 | 6.2 |
| Range | 1-11 | 0.3-17.3 | 0.3-17.3 |
| Duration of constipation-yr | | | |
| Mean | 25.8 | 16.8 | 18.9 |
| Range | 1-65 | 0.5-66.0 | 0.5-66.0 |
| UPDRS score | | | |
| Mean | 53.4 | 63.2 | 61.3 |
| Range | 33-88 | 24-122 | 24.0-122.0 |
| Hoehn and Yahr-Stage | | | |
| Mean | 2.0 | 2.4 | 2.3 |
| Range | 2.0 | 1.0-5.0 | 1.0-5.0 |
| Constipation severity* - | | | |
| CSBM/wk- no. (%) | | | |
| 0-1 | 8 (80) | 14 (41.2) | 22 (50) |
| 1.1-2 | 2 (20) | 17 (50) | 19 (43.2) |
| 2.1-3 | 0 | 3 (8.8) | 3 (6.8) |

*At baseline. Baseline value is the average number of CSBMs per week calculated at the end of the 2-week run-in period.
**In Stage 1, 10 patients received single escalating doses every 3-7 days starting at 25 mg and escalating up to dose limiting toxicity (DLT) or 200 mg, whichever came first, followed by a 2-week wash-out period.
***In Stage 2, 15 patients received daily doses starting at 75 mg and escalating every 3 days up to prokinetic dose (dose producing CSBMs on at least 2 of 3 days) or 175 mg, whichever came first, followed by an additional 2-4 days at that dose (“fixed dose” period) and were then randomized to treatment at the “fixed-dose” or placebo for 4-6 days. Wash-out lasted 2 weeks. The remaining 19 patients were escalated from 100 mg to prokinetic dose or 250 mg, whichever came first, followed by an additional 2-4 days at that dose and then a 2-week wash-out period.

Safety and Adverse Event (AE) Profile:

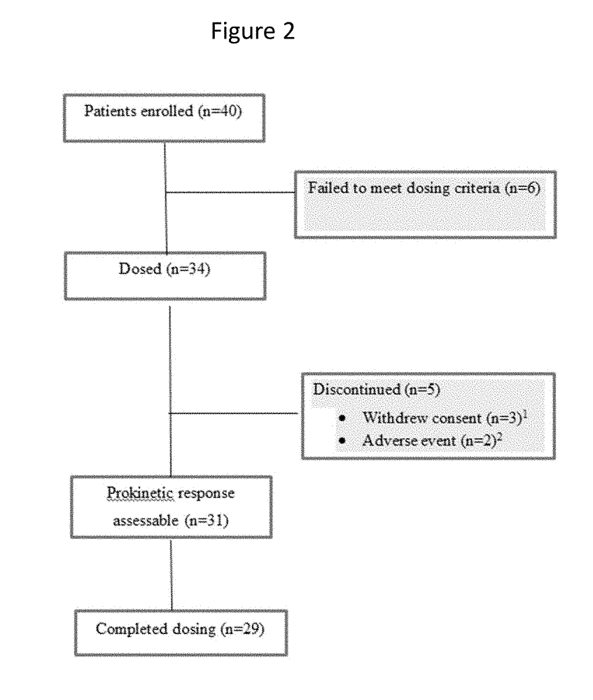

Fifty patients were enrolled and 44 were dosed. In Stage 1, 10 patients were dosed, 1 (10%) withdrew prior to completion and 9 (90%) completed dosing. In stage 2, 6 (15%) patients had ≥3 CSBM/week at the end of the run-in period and were excluded, 34 patients were dosed and bowel response was assessable in 31 (91%). Two patients (5.8%) were terminated prior to completion because of recurrent dizziness, and 3 others withdrew during dosing (8.8%): 2 because of diarrhea and 1 because of holiday. Fifteen patients were randomized. Study-drug assignments and patient disposition are shown in Table 4 and FIG. 2.

TABLE 4
Study drug assignments and adherence to treatment

| | Stage 1 | Stage 2 |
|---|---|---|
| Enrolled | 10 | 40 |
| Failed prior to dosing | 0 | 6 |
| Dosed | 10 | 34 |
| 25-200 mg | 10 | |
| 75-175 mg | | 19 |
| 100-250 mg | | 15 |
| Terminated (%) | 0 (0) | 2* (5.8) |
| Withdrew (%) | 1 (10) | 3 (8.8) |
| Completed dosing (%) | 9 (90) | 31** (91) |
| Randomized | | 15 |
| Treatment | | 6 |
| Placebo | | 9 |

The 2 patients who were terminated **29 patients completed dosing but an additional 2 who withdrew had an assessable prokinetic end-point.

Most AEs were confined to the GI tract (88% in Stage 1 and 63% in Stage 2). The most common AE was nausea which occurred in 4/10 (40%) patients in Stage 1 and in 18/34 (52.9%) in Stage 2 (Table 3). Diarrhea occurred in 4/10 (40%) patients in Stage 1 and 15/34 (44%) in Stage 2. One patient withdrew because of recurrent diarrhea. Other GI related AEs included abdominal pain 11/44 (32%), flatulence 3/44 (6.8%), vomiting 3/44 (6.8%), worsening of acid reflux 2/44 (4.5%), and worsening of hemorrhoids 1/44 (2.2%). One patient had a lower GI bleed (Serious adverse event, SAE) during the withdrawal period. This patient was receiving aspirin, naproxen and clopidogrel at the time of the bleed, and colonoscopy revealed large areas of diverticulosis and polyps. This SAE was considered unrelated to study medication. The only other noteworthy AE was dizziness 8/44 (18%). Dizziness was graded as moderate in one patient who was receiving an alpha-adrenergic blocking agent (Terazosin). This patient was withdrawn from the study and recovered spontaneously. All other AEs resolved spontaneously without discontinuation of squalamine (ENT-01). The relationship between dose and AEs is shown in Table 5.

TABLE 5
All adverse events (n, %)

| | Enrolled | |
|---|---|---|
| | Stage 1 (n = 10) | Stage 2 (n = 40) |
| | Dosed | |
| | 10 | 34 |
| GI: | | |
| Nausea | | |
| Mild | 4 (40) | 18 (52) |
| Moderate | 0 | 1 (2.9) |
| Diarrhea | | |
| Mild | 1 (10) | 12 (35) |
| Moderate | 3 (30) | 2 (5.8) |
| Severe | 0 | 1 (2.9) |
| Vomiting | | |
| Mild | 1 (10) | 2 (5.8) |
| Moderate | 0 | 0 |
| Abdominal pain | | |
| Mild | 2 (20) | 4 (11.7) |
| Moderate | 3 (30) | 2 (5.8) |
| Flatulence | | |
| Mild | 2 (20) | 1 (3) |
| Moderate | 0 | 0 |
| Loss of appetite* | | |
| Mild | 1 (10) | 0 |
| Moderate | 0 | 0 |
| Worsening acid reflux | | |
| Mild | 0 | 4 (11.7) |
| Moderate | 0 | 0 |
| Worsening hemorrhoid | | |
| Mild | 0 | 1 (3) |
| Moderate | 0 | 0 |
| Lower GI bleed** | | |
| Severe | 0 | 1 (2.5) |
| Non-GI: | | |
| Dizziness | | |
| Mild | 0 | 7 (20.5) |
| Moderate | 0 | 1 (2.9) |
| Blood in urine* | | |
| Mild | 1 (10) | 0 |
| Moderate | 0 | 0 |
| Headache | | |
| Mild | 1 (10) | 3 (8.8) |
| Moderate | 0 | 0 |
| Urinary retention | | |
| Mild | 0 | 1 (3) |
| Moderate | 0 | 0 |
| Urinary tract infection | | |
| Mild | 0 | 1 (3) |
| Moderate | 0 | 2 (5.8) |
| Increased urinary frequency | | |
| Mild | 0 | 2 (5.8) |
| Moderate | 0 | 0 |
| Skin lesions-rash | | |
| Mild | 0 | 3 (8.8) |
| Moderate | 0 | 0 |
| Eye infection | | |
| Mild | 0 | 1 (3) |
| Moderate | 0 | 0 |
| Difficulty falling asleep | | |
| Mild | 0 | 1 (3) |
| Moderate | 0 | 0 |

*Unrelated to ENT-01
**colonic diverticulosis, polyp, patient on aspirin, Plavix and naproxen. Unrelated to ENT-01

TABLE 6
Common adverse events by dose

| Dose | Stage 1 | | | Stage 2 | | |
|---|---|---|---|---|---|---|
| (mg) | Diarrhea | Nausea | Vomiting | Diarrhea | Nausea | Dizziness* |
| 0 | 0 | 0 | 0 | 1 | 0 | 2 |
| 25 | 1 | 0 | 0 | — | — | — |
| 50 | 1 | 0 | 0 | — | — | — |
| 75 | 1 | 0 | 0 | 7 | 3 | 8 |
| 100 | 0 | 1 | 1 | 10 | 12 | 7 |
| 125 | 1 | 2 | 1 | 3 | 4 | 8 |
| 150 | 1 | 0 | 0 | 2 | 11 | 2 |
| 175 | 1 | 1 | 0 | 1 | 12 | 0 |
| 200 | 0 | 2 | 0 | 3 | 6 | — |
| 225 | — | — | — | 3 | 1 | |
| 250 | — | — | — | 2 | — | |

*lightheadedness included

TABLE 7
Dose limiting toxicity criteria

| | |
|---|---|
| Diarrhea | Increase 4-6 stools/day over baseline |
| Vomiting | 3-5 episodes in 24 hours |
| Abdominal pain | Moderate pain limiting daily activities |
| Postural hypotension | Moderately symptomatic and limiting daily |
| | activities or BP <80/40 |

No formal sample size calculation was performed for Stage 1. The number of subjects (n=10) was based on feasibility and was considered sufficient to meet the objectives of the study; which was to determine the tolerability of the treatment across the range of tested doses. For Stage 2, assuming the highest proportion of spontaneous resolution of constipation with no treatment to be 0.10, 34 evaluable subjects who have measurements at both baseline and at the end of the fixed dose period provided 80% power to detect the difference between 0.10 (proportion expected if patients are not treated) and a squalamine (ENT-01) treated proportion of 0.29.

No randomization was performed for Stage 1. During the randomization period of Stage 2, subjects were randomly allocated in equal proportion (1:1) to 1 of 2 double-blind treatment groups in a block size of 4: (1) squalamine (ENT-01) at the identified fixed dose level, or (2) placebo at the identified fixed dose level.

Adverse events were coded using the current version of MedDRA. Severity of AEs were assessed by investigators according to CTCAE (v4.03): Grade 1 is labeled as Mild, Grade 2 as Moderate, and Grade 3 and above as Severe. AEs that have a possible, probable or definite relationship to study drug were defined to be related to the study drug while others were defined as “not related”. The number (percentage) of subjects who experienced an AE during escalation and fixed dosing periods were summarized by dose level and overall for each stage. The denominator for calculating the percentages were based on the number of subjects ever exposed to each dose and overall.

Effect on Bowel Function:

Cumulative responder rates of bowel function are shown in FIG. 1A. In Stage 1 (single dose), cumulative response rate increased in a dose-dependent fashion from 25% at 25 mg to a maximum of 80% at 200 mg.

In Stage 2 (daily dosing), the response rate increased in a dose-dependent fashion from 26% at 75 mg to 85.3% at 250 mg. The dose required for a bowel response was patient-specific and varied from 75 mg to 250 mg. Median efficacious dose was 100 mg. Average CSBM/week increased from 1.2 at baseline to 3.8 at fixed dose (p=2.3×10⁻⁸) and SBM increased from 2.6 at baseline to 4.5 at fixed dose (p=6.4×10⁻⁶) (Table 8). Use of rescue medication decreased from 1.8/week at baseline to 0.3 at fixed dose (p=1.33×10⁻⁵). Consistency based on the Bristol stool scale also improved, increasing from mean 2.7 to 4.1 (p=0.0001) and ease of passage increased from 3.2 to 3.7 (p=0.03). Subjective indices of wellbeing (PAC-QOL) and constipation symptoms (PAC-SYM) also improved during treatment (p=0.009 and p=0.03 respectively).

TABLE 8
Stool related indices Stage 2 (Dosed patients, n = 34)

| | | Fixed dose | |
|---|---|---|---|
| | Baseline (mean, SD) | (mean, SD) | P-value |
| CSBM* | 1.2 (0.90) | 3.8 (2.40) | 2.3 × 10⁻⁸ |
| SBM* | 2.6 (1.45) | 4.5 (2.21) | 6.4 × 10⁻⁶ |
| Suppository use* | 1.8 (1.92) | 0.3 (0.67) | 1.33 × 10⁻⁵ |
| Consistency*** | 2.7 (1.20) | 4.1 (2.13) | 0.0001 |
| Ease of passage** | 3.2 (0.73) | 3.7 (1.19) | 0.03 |
| PAC-QOL total | 1.4 (0.49) | 1.2 (0.59) | 0.009 |
| PAC-SYM | 1.3 (0.45) | 1.1 (0.49) | 0.03 |

*weekly average;
**Ease of evacuation scale, where 1—manual disimpaction and 7 = incontinent;
***Bristol stool scale 1-7, where 1 = separate hard lumps and 7 = liquid consistency

The dose that proved efficacious in inducing a bowel response was strongly related to constipation severity at baseline (p=0.00055) (FIG. 1B); patients with baseline constipation of <1 CSBM/week required higher doses for a response (mean 192 mg) than patients with ≥1 CSBM/week (mean 120 mg).

While the improvement in most stool-related indices did not persist beyond the treatment period, CSBM frequency remained significantly above baseline value (Table 9).

TABLE 9
Reversal of stool indices to baseline during the wash-out period (Stage 2)

| | | | | P-value |
|---|---|---|---|---|
| | Baseline | Fixed dose | Wash-out | (wash-out vs. |
| | (Mean, SD) | (Mean, SD) | (Mean, SD) | baseline) |
| CSBM | 1.2 (0.90) | 3.8 (2.4) | 1.8 (1.19) | 0.01 |
| SBM | 2.6 (1.45) | 4.5 (2.21) | 3.2 (1.80) | 0.16 |
| Ease | 3.2 (0.73) | 3.7 (1.19) | 3.3 (0.81) | 0.78 |
| Consistency | 2.7 (1.20) | 4.1 (2.13) | 2.8 (1.39) | 0.85 |
| Rescue meds | 1.8 (1.92) | 0.3 (0.67) | 1.0 (1.40) | 0.13 |
| PAQ-QOL | 1.4 (0.49) | 1.2 (0.59 | 1.2 (0.63) | 0.04 |
| PAQ-SYM | 1.3 (0.45) | 1.1 (0.49) | 1.1 (0.60) | 0.11 |

The primary efficacy outcome variable was whether or not a subject was a “success” or “failure”. This is an endpoint based on subject diary entries for the “fixed dose” period prior to the endpoint assessment defined as average complete stool frequency increase by 1 or more over baseline, or 3 or more complete spontaneous stools/week. The subject was deemed a “success” if s/he met one or more of the criteria listed above, otherwise the subject was deemed a “failure”. The primary analysis was based on all subjects with a baseline assessment and an assessment at the end of the “fixed-dose” period and was a comparison of the proportion of successes with 0.10 (the null hypothesis corresponding to no treatment effect).

The proportion of subjects for whom the drug was a success was estimated with a binomial point estimate and corresponding 95% confidence interval. A secondary analysis compared the proportions of subjects who are deemed a success at the end of the randomized fixed-dose period between those randomized to the squalamine (ENT-01) arm and those randomized to the placebo arm. A Fisher's exact test was used to compare the proportions of subjects who were deemed a success at the end of randomization period between the two randomized arms.

Subgroup Analysis:

Fifteen patients were randomized to treatment (n=6) or placebo (n=9) after the fixed dose period. During the 4-6 days of randomized treatment, the mean CSBM frequency in the treatment group remained higher than baseline as compared to those receiving placebo who returned to their baseline values (Table 10).

TABLE 10
CSBM frequency in the randomized cohort

| CSBM/week | Baseline | Fixed dose | Randomized | Washout |
|---|---|---|---|---|
| Treatment (n = 6) | 0.8 | 3.2 | 2.4 | 0.9 |
| Placebo (n = 9) | 1.6 | 3.3 | 1.4 | 1.6 |

CSBM increased in both groups during the treatment period and remained high in the treatment group during the randomized period but fell to baseline values in the placebo group.

Pharmacokinetics:

PK data were collected on the 10 patients enrolled in Stage 1 and 10 patients enrolled in Stage 2 to determine the extent of systemic absorption. In Stage 1, PK data were obtained at each visit, pre-medication, at 1, 2, 4, 8 and 24 hours (Table 11). In Stage 2, PK was measured on days 1 and 6 of the randomization period pre-medication, at 1, 2, 4 and 8 hours (Table 12). Based on the pharmacokinetic behavior of intravenously administered squalamine determined in prior clinical studies it is estimated that squalamine (ENT-01) exhibited oral bio-availability of less than 0.3% (Bhargava et al. 2001; Hao et al. 2003).

TABLE 11
Pharmacokinetics of orally administered squalamine (ENT-01) in Stage 1.
Stage 1

| | | | Tₘₐₓ (hour) | T½ | | |
|---|---|---|---|---|---|---|
| Dose | # of | Cₘₐₓ | (Median | (hours) | AUC₀₋₈ₕᵣ | AUC₀₋₁₆ₕᵣ |
| (mg) | patients | (ng/ml) | Value) | (n) | (ng*hour/ml | (ng*hour/ml |
| 25 | 9 | 2.84 | 1.0 | 2.6 (3) | 10.8 | 19.6 |
| 50 | 10 | 3.73 | 2.0 | 3.4 (3) | 18.5 | 33.1 |
| 75 | 9 | 4.33 | 2.0 | 2.8 (2) | 18.4 | 29.8 |
| 100 | 9 | 6.18 | 2.0 | 3.9 (5) | 29.6 | 51.5 |
| 125 | 9 | 9.63 | 2.0 | 3.9 (4) | 43.1 | 77.7 |
| 150 | 7 | 6.27 | 2.0 | 5.6 (4) | 31.5 | 64.0 |
| 175 | 7 | 10.3 | 2.0 | 9.1 (6) | 49.7 | 91.2 |
| 200 | 6 | 15.1 | 2.0 | 9.0 (5) | 78.3 | 157 |

TABLE 12
Pharmacokinetics of orally administered squalamine (ENT-01) in Stage 2.
Stage 2

| | # of | | | | |
|---|---|---|---|---|---|
| | patients | | Tₘₐₓ (hour) | T_{1/2} | |
| Dose | (2 visits | Cₘₐₓ | (Median | (hours) | AUC_{0-8 hr} |
| (mg) | each) | (ng/ml) | Value) | (n) | (ng * hour/ml |
| 75 | 1 | 10.0 | 3.0 | 5.5 (1) | 59.0 |
| 100 | 4 | 17.7 | 1.0 | 4.8 (5) | 70.3 |
| 125 | | | | | |
| 150 | | | | | |
| 175 | 5 | 11.8 | 2.0 | 10 (6) | 66.8 |

The mean Cₘₐₓ, Tₘₐₓ and T_{1/2} and AUC of the squalamine ion following squalamine (ENT-01) oral dosing for Stage 1 patients. The PK analyses are only approximate, as the lower limit of the validated concentration range was 10 ng/ml; most of the measured concentrations fell below that value. The mean Cₘₐₓ, Tₘₐₓ and T_{1/2} and AUC of the squalamine ion following squalamine (ENT-01) oral dosing for Stage 2 patients. The PK analyses are only approximate, as the lower limit of the validated concentration range was 0.5 ng/ml.

CNS Symptoms in Stage 2:

An exploratory analysis was done with respect to the sleep data, the body temperature data, mood, fatigue, hallucinations, cognition and other motor and non-motor symptoms of PD. Continuous measurements within a subject were compared with a paired t-test and continuous measurements between subject groups were compared with a two-group t-test. Categorical data were compared with a chi-squared test or a Fisher's exact test if the expected cell counts are too small for a chi-squared test.

CNS symptoms:

CNS symptoms were evaluated at baseline and at the end of the fixed dose period and the wash-out period (Table 13). Total UPDRS score was 64.4 at baseline, 60.6 at the end of the fixed dose period and 55.7 at the end of the wash-out period (p=0.002); similarly, the motor component of the UPDRS improved from 35.3 at baseline to 33.3 at the end of fixed dose to 30.2 at the end of wash-out (p=0.006). MMSE improved from 28.4 at baseline to 28.7 during treatment and to 29.3 during wash-out (p=0.0006). BDI-II decreased from 10.9 at baseline to 9.9 during treatment and 8.7 at wash-out (p=0.10). PDHQ improved from 1.3 at baseline to 1.8 during treatment and 0.9 during wash-out (p=0.03). Hallucinations were reported by 5 patients at baseline and delusions in 1 patient. Both hallucinations and delusions improved or disappeared in 5 of 6 patients during treatment and did not return for 4 weeks following discontinuation of squalamine (ENT-01) in 1 patient and 2 weeks in another. The frequency of arm or leg thrashing reported in the sleep diary diminished progressively from 2.2 episodes/week at baseline to 0 at maximal dose. Total sleep time increased progressively from 7.1 hours at baseline to 8.4 hours at 250 mg and was consistently higher than baseline beyond 125 mg (FIGS. 3-5). Unlike stool-related indices, the improvement in many CNS symptoms persisted during wash-out.

TABLE 13
Effect of Squalamine (ENT-01) on neurological symptoms (n = 34)

| | | | | Wash-out | |
|---|---|---|---|---|---|
| | Baseline | Fixed dose | | (Mean, | |
| UPDRS | (Mean, SD) | (Mean, SD) | P-value | SD) | P-value |
| Part 1 | 11.6 (6.51) | 10.6 (6.18)) | 0.28 | 9.5 (5.27) | 0.06 |
| (NMS) | | | | | |
| Part 2 | 14.9 (8.11) | 14.7 (9.02) | 0.77 | 14.1 (8.21) | 0.40 |
| (Daily | | | | | |
| living) | | | | | |
| Part 3 | 35.3 (14.35) | 33.3 (15.20) | 0.13 | 30.2 (13.23) | 0.005 |
| (Motor) | | | | | |
| Total | 64.4 (23.72) | 60.6 (25.60) | 0.09 | 55.7 (23.69) | 0.002 |
| MMSE | 28.4 (1.75) | 28.7 (1.9) | 0.21 | 29.3 (1.06) | 0.0006 |
| PDHQ | 1.3 (2.99) | 1.8 (3.34) | 0.45 | 0.9 (2.33) | 0.03 |
| BDI-II | 10.9 (7.12) | 9.9 (6.45) | 0.14 | 8.7 (5.19) | 0.10 |

UPDRS: Unified Parkinson's Disease Severity Score;
NMS: Non-motor symptoms;
BDI: Beck Depression Index-II;
MMSE: Mini-mental State exam.
PDHQ: Parkinson's Disease Hallucination Questionnaire

Circadian rhythm of skin temperature was evaluable in 12 patients (i.e., those who had recordings that extended from baseline through washout). Circadian system functionality was evaluated by continuously monitoring wrist skin temperature using a temperature sensor (Thermochron iButton DS1921H; Maxim, Dallas, Tex.) (Sarabia et al. 2008). A nonparametric analysis was performed for each participant to characterize DST as previously described (Sarabia et al. 2008; Ortiz-Tudela et al. 2010).

Briefly, this analysis includes the following parameters: (i) the inter-daily stability (the constancy of 24-hour rhythmic pattern over days, IS); (ii) intra-daily variability (rhythm fragmentation, IV); (iii) average of 10-minute intervals for the 10 hours with the minimum temperature (L10); (iv) average of 10-minute intervals for the 5 hours with the maximum temperature (M5) and the relative amplitude (RA), which was determined by the difference between M5 and L10, divided by the sum of both. Finally, the Circadian Function Index (CFI) was calculated by integrating IS, IV, and RA. Consequently, CFI is a global measure that oscillates between 0 for the absence of circadian rhythmicity and 1 for a robust circadian rhythm (Ortiz-Tudela et al. 2010).

A comparison was performed of circadian rhythm parameters during the baseline, fixed dose and washout periods. ENT-01 administration improved all markers of healthy circadian function, increasing rhythm stability (IS, p=0.026), relative amplitude (RA, p=0.001) and circadian function index (CFI, p=0.016), while reducing rhythm fragmentation (IV, p=0.031). The improvement persisted for several of these circadian parameters during wash-out period (IS, p=0.008 and CFI, p=0.004). (FIG. 6).

### CONCLUSIONS

This Phase 2 trial involving 50 patients with PD assessed the safety of orally administered ENT-01, and the effect on bowel function and neurologic symptoms of PD. In addition, the study aimed to identify a dose of ENT-01 that normalizes bowel function in each patient. The study achieved the objectives of identifying safety and pharmacodynamics responses of ENT-01 in PD. In addition, the study is the first proof of concept demonstration that directly targeting αS pharmacologically can achieve beneficial GI, autonomic and CNS responses.

The effective dose ranged between 75 mg and 250 mg, with 85% of patients responding within this range. This dose correlated positively with constipation severity at baseline consistent with the hypothesis that gastrointestinal dysmotility in PD results from the progressive accumulation of αS in the ENS, and that squalamine (ENT-01) can restore neuronal function by displacing αS and stimulating enteric neurons. These results demonstrate that the ENS in PD is not irreversibly damaged and can be restored to normal function.

Several exploratory endpoints were incorporated into the trial to evaluate the impact of ENT-01 on neurologic symptoms associated with PD. The UPDRS score, a global assessment of motor and non-motor symptoms, showed significant improvement. Improvement was also seen in the motor component. The improvement in the motor component is unlikely to be due to improved gastric motility and increased absorption of dopaminergic medications, since improvement persisted during the 2-week wash-out period, i.e., in the absence of study drug.

Improvements were also seen in cognitive function (MMSE scores), hallucinations, REM-behavior disorder (RBD) and sleep. Six of the patients enrolled had daily hallucinations or delusions and these improved or disappeared during treatment in five. In one patient the hallucinations disappeared at 100 mg, despite not having reached the colonic prokinetic dose at 175 mg. The patient remained free of hallucinations for 1 month following cessation of dosing. RBD and total sleep time also improved progressively in a dose-dependent manner.

The prokinetic effect of the amino sterol squalamine appears to occur through local action of the compound on the ENS, since squalamine, the active zwitterion, is not significantly absorbed into the systemic circulation.

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, or compositions, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof, inclusive of the endpoints. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims.

### REFERENCES

1. Aarsland et al., “Neuropsychiatric symptoms in patients with Parkinson's disease and dementia: frequency, profile and associated care giver stress,” J. Neurol. Neurosurg. Psychiatry, 78:36-42 (2007).
2. Acsadi et al., “Alpha-synuclein loss in spinal muscular atrophy,” J. Mol. Neurosci., 43(3):275-83 (2011).
3. Albert et al., “The Diagnosis of Mild Cognitive Impairment Due to Alzheimer's Disease: Recommendations from the National Institute on Aging—Alzheimer's Association Workgroups on Diagnostic Guidelines for Alzheimer's Disease,” Alzheimer's & Dementia, 7(3):270-279 (2011).
4. Alexander et al., “Membrane surface charge dictates the structure and function of the epithelial na+/h+exchanger.” EMBO J., 30:679-691 (2011).
5. Andresen et al., “Effect of 5 days linaclotide on transit and bowel function in females with constipation-predominant irritable bowel syndrome,” Gastroenterology, 133:761-8 (2007).
6. Antonelou et al., “Decreased levels of alpha-synuclein in cerebrospinal fluid of patients with clinically isolated syndrome and multiple sclerosis,” J. Neurochem., 134(4):748-55 (2015).
7. Antonio-Rubio et al., “Abnormal thermography in Parkinson's disease,” Parkinsonism Relat. Disord., 21:852-7 (2015).
8. Auyeung et al., “Ten year survival and outcomes in a prospective cohort of new onset Chinese Parkinson's disease patients,” J. Neurol. Neurosurg. Psychiatry, 83:607-11 (2012).
9. Bassiony et al., “Delusions and Hallucinations in Alzheimer's Disease: Review of the Brain Decade,” Psychosomatics, 44(5):388-401) 2003).
10. Beghi et al., “The epidemiology of ALS and the role of population-based registries,” Biochim. Biophys. Acta, 1762: 1150-1157 (2006).
11. K. Bertram and D. Williams, “Visual hallucinations in the differential diagnosis of parkinsonism,” J. Neurol. Neurosurg. Psychiatry, Published Online First: 6 Jan. 2012. doi: 10.1136/jnnp-2011-300980.
12. Bhargava et al., “A phase I and pharmacokinetic study of squalamine, a novel antiangiogenic agent, in patients with advanced cancers.” Clin. Cancer Res., 7(12): 3912-9 (2001).
13. Braak et al., “Idiopathic Parkinson's disease: possible routes by which vulnerable neuronal types may be subject to neuroinvasion by an unknown pathogen,” J Neural Transm (Vienna), 110:517-36 (2003).
14. Breen et al., “Sleep and circadian rhythm regulation in early Parkinson disease,” JAMA Neurol., 71:589-95 (2014).
15. Brundin et al., “Therapeutic approaches to target alpha-synuclein pathology,” Exp. Neurology, 298(B):225-235 (2017).
16. Burghaus et al., “Hallucinations in neurodegenerative diseases,” CNS Neurosci. Ther., 18(2):149-59 (2012).
17. Burre et al., “Cell Biology and Pathophysiology of α-Synuclein.” Cold Spring Harbour Perspectives Medicine, 8: pii:a024091 (2018).
18. D. Collerton and J. Taylor, “Advances in the treatment of visual hallucinations in neurodegenerative diseases,” Future Neurol., 8(4):433-444 (2013).
19. Connolly et al., “Squalamine lactate for exudative age-related macular degeneration,” Ophthalmol. Clin. North Am., 19:381-91 (2006).
20. Cordell et al., “Alzheimer's Association Recommendations for Operationalizing the Detection of Cognitive Impairment During the Medicare Annual Wellness Visit in a Primary Care Setting,” Alzheimer's & Dementia, 9(2):141-150 (2013).
21. Correa et al., “Association of Huntington's disease and schizophrenia-like psychosis in a Huntington's disease pedigree,” Clin. Pract. Epidemiol. Ment. Health, 2:1 (2006) (https://www.ncbi.nlm.nih.gov/pmc/articles/PMC1386660/).
22. Corrochano et al., “α-synuclein levels affect autophagosome numbers in vivo and modulate Huntington disease pathology,” Autophagy, 8(3):431-432 (March 2012).
23. Corrochano et al., “α-synuclein levels modulate Huntington's disease in mice,” Hum. Mol. Genet., 21(3):485-94 (February 2012).
24. Demirel et al., “Decreased Expression of α-Synuclein, Nogo-A and UCH-L1 in Patients with Schizophrenia: A Preliminary Serum Study,” Psychiatry Invest., 14(3) (2017).
25. Diederich et al., “Hallucinations in Parkinson disease,” Nat. Rev. Neurol., 5(6):331-42 (2009).
26. Dogan et al., “Cognition in Friedreich's ataxia: a behavior and multimodal imaging study,” Ann. Clin. Transl. Neurol., 3(8):572-587 (2016).
27. Emin Ozcan et al., “Multiple Sclerosis Presents with Psychotic Symptoms and Coexists with Hypertrophic Cardiomyopathy,” Case Reports in Neurological Med., vol. 2014, 4 pages, Article ID 383108 (http://dx.doi.org/10.1155/2014/383108).
28. Erskine et al., “Neuronal Loss and A-Synuclein Pathology in the Superior Colliculus and Its Relationship to Visual Hallucinations in Dementia with Lewy Bodies.” Am J Geriatr Psychiatry 25: 595-604 (2017).
29. Fahn S ER, Members of the UPDRS Development Committee. UNIFIED PARKINSON'S DISEASE RATING SCALE. Florham Park, N.J.: Macmillan Health Care Information (1987).
30. Fenelon et al., “Hallucinations in Parkinson's disease: Prevalence, phenomenology and risk factors,” Brain, 123(4):733-745 (Apr. 1, 2000).
31. Frank et al., “Psychometric validation of a constipation symptom assessment questionnaire,” Scand. J. Gastroenterol., 34:870-7 (1999).
32. Friedman et al., “Multisensory Hallucinations and Delusions in Parkinson's Disease,” Scientific Issues Committee Blog, May 2018 (International Parkinson and Movement Disorder Society) (https://www.movementdisorders.org/MDS/Scientific-Issues-Committee-Blog-Multisensory-Hallucinations-and-Delusions-in-Parkinsons-Disease.htm, downloaded on Oct. 19, 2018).
33. Frieling et al., “Alpha-synuclein mRNA levels correspond to beck depression inventory scores in females with eating disorders,” Neuropsychobiology, 58(1):48-52 (2008).
34. Frisina et al., “The neuropathological basis for depression in Parkinson's disease,” Parkinsonism Relat. Disord., 15(2):144-148 (2009).
35. FTD Talk 2015, “Psychosis is more common than previously thought in frontotemporal dementia” (Aug. 3, 2015).
36. Gilberthorpe et al., “The spectrum of psychosis in multiple sclerosis: a clinical case series,” Neuropsychiatr. Dis. Treat., 13:303-318 (2017).
37. Gjerstad et al., “Excessive daytime sleepiness in Parkinson disease: is it the drugs or the disease?” Neurology, 67:853-8 (2006).
38. Goetz C G, Stebbins G T., “Risk factors for nursing home placement in advanced Parkinson's disease,” Neurology, 43:2227-9 (1993).
39. Gressens et al., “Vasoactive intestinal peptide prevents excitotoxic cell death in the murine developing brain,” J. Clin. Invest., 100(2): 390-7 (1997).
40. Hao et al., “A Phase I and pharmacokinetic study of squalamine, an amino sterol angiogenesis inhibitor,” Clin. Cancer Res., 9(7): 2465-71 (2003).
41. Heaton et al., Defecation frequency and timing, and stool form in the general population: a prospective study. Gut, 33:818-24 (1992).
42. J. Hinkle and G. Pontone, “Visual Hallucinations in Neurodegenerative Diseases: Focus on Selective Tissue Vulnerability,” Am. J. of Geriatric Psychiatry, 25(6):605-606 (June 2017).
43. Holmqvist et al., “Direct evidence of Parkinson pathology spread from the gastrointestinal tract to the brain in rats,” Acta Neuropathol., 128:805-20 (2014).
44. Hosokawa et al., “Accumulation of multiple neurodegenerative disease-related proteins in familial frontotemporal lobar degeneration associated with granulin mutation,” Scientific Reports, 7:1513 (2017).
45. Hughes et al., “Accuracy of clinical diagnosis of idiopathic Parkinson's disease: a clinico-pathological study of 100 cases,” J. Neurol. Neurosurg. Psychiatry, 55:181-4 (1992).
46. Jack et al., “Introduction to the Recommendations from the National Institute on Aging—Alzheimer's Association Workgroups on Diagnostic Guidelines for Alzheimer's Disease,” Alzheimer's & Dementia, 7(3):257-262 (2011).
47. Jorm, A F, “The Informant Questionnaire on cognitive decline in the elderly (IQCODE): a review,” Int. Psychogeriatr., 16(3):275-93 (2004).
48. Kim et al., “Poststroke Induction of α-Synuclein Mediates Ischemic Brain Damage,” J. Neurosci., 36(26):7055-7065 (June 2016).
49. Kirkevold, O. & Selbaek, G., “The Agreement Between the MMSE and IQCODE Tests in a Community-Based Sample of Subjects Aged 70 Years or Older Receiving In-Home Nursing: An Explorative Study,” Dement Geriatr. Cogn. Dis. Extra, 5(1):32-41 (2015).
50. Kline et al., “Comparison of independent screens on differentially vulnerable motor neurons reveals alpha-synuclein as a common modifier in motor neuron diseases,” PLOS Genetics, 13(3):e1006680 (2017).
51. Koh et al., “Cerebral ischemic injury decreases α-synuclein expression in brain tissue and glutamate-exposed HT22 cells,” Lab. Anim. Res., 33(3):244-250 (September 2017).
52. Kokubo et al., “α-Synuclein Pathology in the Amyotrophic Lateral Sclerosis/Parkinsonism Dementia Complex in the Kii Peninsula, Japan,” J. of Neuropathology & Exp. Neurology, 7/(7):625-630 (2012).
53. Landqvist Waldo et al., “Psychotic symptoms in frontotemporal dementia: a diagnostic dilemma?” Int. Psychogeriatrics, 27(4):531-539 (April 2015).
54. Lewis et al., “Stool form scale as a useful guide to intestinal transit time,” Scand. J. Gastroenterol, 32:920-4 (1997).
55. Li et al., “A splitting brain: Imbalanced neural networks in schizophrenia,” Psychiatry Res., 232(2):145-53 (May 30, 2015).
56. Lin et al., “Risk of Parkinson's disease following severe constipation: a nationwide population-based cohort study,” Parkinsonism Relat Disord, 20:1371-5 (2014).
57. Llorca et al., “Hallucinations in schizophrenia and Parkinson's disease: an analysis of sensory modalities involved and the repercussion on patients,” Sci. Rep., 6:38152 (2016).
58. Madrid-Navarro et al., “Multidimensional Circadian Monitoring by Wearable Biosensors in Parkinson's Disease,” Front. Neurol., 9:157 (2018).
59. Marquis et al., Development and validation of the Patient Assessment of Constipation Quality of Life questionnaire. Scand J Gastroenterol, 40:540-51 (2005)
60. McDowell et al., “Sleep dysfunction and EEG alterations in mice overexpressing alpha-synuclein,” J. Parkinsons Dis., 4(3):531-539 (2014).
61. McKhann et al., “The Diagnosis of Dementia Due to Alzheimer's Disease: Recommendations from the National Institute on Aging—Alzheimer's Association Workgroups on Diagnostic Guidelines for Alzheimer's Disease,” Alzheimer's & Dementia, 7(3):263-269 (2011).
62. Mearin et al., “Bowel Disorders,” Gastroenterology, 150(6):1393-1407 (2016).
63. Mejia et al., “Low Levels of Alpha-Synuclein in Peripheral Tissues Are Related to Clinical Relapse in Relapsing-Remitting Multiple Sclerosis (P1.392),” Neurology, 90(15 Supplement) (Apr. 10, 2018).
64. E. Milne, “Autistic people are three times more likely to have hallucinations,” The Conversation (May 18, 2017) (https://theconversation.com/autistic-people-are-three-times-more-likely-to-have-hallucinations-77337).
65. Miraglia et al., “Parkinson's disease and alpha-synucleinopathies: from arising pathways to therapeutic challenge,” Cen.t Nerv. Syst. Agents Med. Chem., 15: 109-16 (2015).
66. Moussaud et al., “Alpha-synuclein and tau: teammates in neurodegeneration?” Mol. Neurodeg., 9:43 (2014).
67. O'Brien et al., “Clustering of Neuropsychiatric Disease in First-Degree and Second-Degree Relatives of Patients with Amyotrophic Lateral Sclerosis,” JAMA Neurol., 74(12):1425-1430 (Dec. 1, 2017).
68. Ondo et al., “Daytime sleepiness and other sleep disorders in Parkinson's disease,” Neurology, 57:1392-6 (2001).
69. Ondo et al., “Placebo-controlled trial of lubiprostone for constipation associated with Parkinson disease,” Neurology, 78:1650-4 (2012).
70. Ortiz-Tudela, E., Martinez-Nicolas, A., Campos, M., Rol, M. A. & Madrid, J. A. “A new integrated variable based on thermometry, actimetry and body position (TAP) to evaluate circadian system status in humans,” PLoS Computational Biology 6, e1000996, https://doi.org/10.1371/journal.pcbi.1000996 (2010).
71. Ortiz-Tudela et al., “Ambulatory circadian monitoring (ACM) based on thermometry, motor activity and body position (TAP): a comparison with polysomnography,” Physiol. Behav., 126:30-8 (2014).
72. Palsetia et al., “The Clock Drawing Test versus Mini-mental Status Examination as a Screening Tool for Dementia: A Clinical Comparison,” Indian J. Psychol. Med., 40:1-10 (2018).
73. Papapetropoulos et al., “A questionnaire-based (UM-PDHQ) study of hallucinations in Parkinson's disease,” BMC Neurol., 8:21 (2008).
74. Perni et al., “A natural product inhibits the initiation of alpha-synuclein aggregation and suppresses its toxicity,” PNAS, USA, 114:E1009-E17 (2017).
75. Phillips et al., “Alpha-synuclein-immunopositive myenteric neurons and vagal preganglionic terminals: autonomic pathway implicated in Parkinson's disease?” Neuroscience, 153:733-50 (2008).
76. Phukan et al., “Cognitive impairment in amyotrophic lateral sclerosis,” Lancet Neurol., 6(11):994-1003 (2007).
77. Plassman et al., “Prevalance of Cognitive Impairment Without Dementia in the United States,” Ann. Intern. Med., 148(6):427-434 (2009).
78. Rahn et al., “Cognitive Impairment in Multiple Sclerosis: A Forgotten Disability Remembered,” Cerebrum., 201214 (November-December 2012).
79. Rao et al., “Amino sterols from the dogfish shark Squalus acanthias,” J. Nat. Prod., 63(5): 631-5 (2000).
80. Reeve et al., “Ageing and Parkinson's disease: why is advancing age the biggest risk factor?” Ageing Res. Rev., 14: 19-30 (2014).
81. Reitz et al., “Epidemiology of Alzheimer disease,” Nat. Rev. Neurol., 7: 137-152 (2011).
82. E. Rubin, “Cognitive Impairment in Depression,” Psychology Today (Jun. 8, 2016)
83. Sarabia et al., “Circadian rhythm of wrist temperature in normal-living subjects A candidate of new index of the circadian system,” Physiol. Behav., 95:570-80 (2008).
84. Senadim et al., “Visual Hallucinations and Ischemic Stroke: Review of 5 Cases,” J. Neurol. Neurophysiol., 8:440 (2017).
85. Shehata et al., “Neuronal stimulation induces autophagy in hippocampal neurons that is involved in AMPA receptor degradation after chemical long-term depression,” J. Neurosci., 32:10413-22 (2012)
86. W. Sriwimol and P. Limprasert, “Significant Changes in Plasma Alpha-Synuclein and Beta-Synuclein Levels in Male Children with Autism Spectrum Disorder,” BioMed Res. Int., Vol. 2018, Article ID 4503871 (Apr. 8, 2018).
87. Steer et al., “Use of the Beck Depression Inventory-II with depressed geriatric inpatients,” Behav. Res. Ther., 38:311-8 (2000).
88. Stiasny-Kolster et al., “The REM sleep behavior disorder screening questionnaire—a new diagnostic instrument,” Movement disorders: Official J. of the Movement Dis. Soc., 22:2386-93 (2007).
89. Stoessl, Jon, “Neuroimaging in the early diagnosis of neurodegenerative disease,” Transl. Neurodegener., 1:5 (2012).
90. Sumioka et al., “TARP phosphorylation regulates synaptic AMPA receptors through lipid bilayers,” Neuron, 66(5): 755-67 (2009).
91. Svensson et al., “Does vagotomy reduce the risk of Parkinson's disease: The authors reply” Ann. Neurol., 78:1012-3 (2015).
92. Tang et al., “Loss of mTOR-Dependent Macroautophagy Causes Autistic-like Synaptic Pruning Deficits,” Neuron, 83(5):1131-1143 (2014).
93. United Nations. World population ageing 2013. United Nations. Department of Economic and Social Affairs Population Division; Available online at: http://www.un.org/en/development/desa/population/publications/pdf/ageing/WorldPopulationAgeingReport2013.pdf).
94. U.S. Pat. No. 5,192,756 for “Aminosterol antibiotic,” Zasloff, Moore, Wehrli, Issued Mar. 9, 1993.
95. U.S. Pat. No. 5,840,936 (1998) for “Aminosterol compounds useful as inhibitors of the sodium/proton exchanger (NHE),” Zasloff, Shinnar, Rao, Kinney.
96. U.S. Pat. No. 6,962,909 (2005) for “Treatment of neovascularization disorders with squalamine,” Zasloff, Shinnar, Kinney, Jones, Issued Nov. 8, 2005.
97. Videnovic A, Golombek D., “Circadian Dysregulation in Parkinson's Disease,” Neurobiol. Sleep Circadian Rhythms, 2:53-8 (2017).
98. Wang et al., “Shared and Specific Intrinsic Functional Connectivity Patterns in Unmedicated Bipolar Disorder and Major Depressive Disorder,” Sci. Rep., 7(1):3570 (Jun. 15, 2017).
99. West, et al., “Squalamine increases vagal afferent firing frequency in aging mice,” J. of the Canadian Association of Gastroenterology, 1 (2018).
100. Yancopoulou et al., “Tau and alpha-synuclein inclusions in a case of familial frontotemporal dementia and progressive aphasia,” J. Neuropathol. Exp. Neurol., 64(3):245-53 (2005).
101. Yeung et al., “Membrane phosphatidylserine regulates surface charge and protein localization,” Science, 319(5860): 210-3 (2008).
102. Yee et al., “Persistent hallucinosis in borderline personality disorder,” Compr Psychiatry; 46:147-54 (2005).
103. Yun et al., “Identification of Squalamine in the Plasma Membrane of White Blood Cells in the Sea Lamprey,” Petromyzon marinus, “J. Lipid Res., 48(12): 2579-2586 (2007).
104. Zahodne et al., “Mood and motor trajectories in Parkinson's disease: multivariate latent growth curve modeling,” Neuropsychology, 26:71-80 (2012).
105. Zasloff et al., “Squalamine as a broad-spectrum systemic antiviral agent with therapeutic potential,” Proc. Natl. Acad. Sci. USA, 108(38): 15978-83 (2011).
106. Zhao et al., “A comparative study of the amount of α-synuclein in ischemic stroke and Parkinson's disease,” Neurol. Sci., 37(5):749-54 (May 2016).
107. Zinsmeister et al., “Pharmacodynamic and clinical endpoints for functional colonic disorders: statistical considerations,” Dig. Dis. Sci., 58:509-18 (2013).

What is claimed is:

1. A method of treating, preventing and/or slowing the onset or progression of hallucinations and/or a related symptom in a subject in need comprising:
(a) selecting a subject suffering from or potentially susceptible to hallucinations; and
(b) administering to the subject a therapeutically effective amount of at least one aminosterol or a salt or derivative thereof.

2. The method of claim 1, wherein the therapeutically effective amount of the at least one aminosterol or a salt or derivative thereof:
(a) comprises about 0.001 to about 500 mg per day; and/or
(b) comprises about 0.001 to about 500 mg per day, about 0.001 to about 375 mg per day, about 0.001 to about 250 mg per day, or about 0.001 to about 125 mg per day; or
(c) comprises about 0.1 to about 20 mg/kg body weight of the subject.

3. The method of claim 1, wherein the method of administration comprises nasal administration and the therapeutically effective amount of the at least one amino sterol, or a salt or derivative thereof comprises:
(a) about 0.001 to about 6 mg per day; or
(b) about 0.001 to about 4 mg per day.

4. The method of claim 1, wherein the administration comprises oral administration and the therapeutically effective amount of the at least one amino sterol, or a salt or derivative thereof comprises:
(a) about 1 to about 300 mg per day; or
(b) about 25 to about 300 mg per day.

5. A method of treating, preventing and/or slowing the onset or progression of hallucinations and/or a related symptom in a subject in need comprising:
(a) determining a dose of an aminosterol or a salt or derivative thereof for the subject, wherein the aminosterol dose is determined based on the effectiveness of the aminosterol dose in improving or resolving a hallucination symptom being evaluated,
(b) followed by administering the dose of the aminosterol or a salt or derivative thereof to the subject for a defined period of time, wherein the method comprises:
(i) identifying a hallucination symptom to be evaluated;
(ii) identifying a starting dose of an aminosterol or a salt or derivative thereof for the subject; and
(iii) administering an escalating dose of the amino sterol or a salt or derivative thereof to the subject over a defined period of time until an effective dose for the hallucination symptom being evaluated is identified, wherein the effective dose is the amino sterol dose where improvement or resolution of the hallucination symptom is observed, and fixing the aminosterol dose at that level for that particular hallucination symptom in that particular subject.

6. The method of claim 5, wherein the amino sterol or a salt or derivative thereof is a pharmaceutically acceptable grade of the amino sterol or a salt or derivative thereof.

7. The method of claim 5, wherein:
(a) the hallucinations are correlated with abnormal αS pathology; and/or
(b) the hallucinations are correlated with dopaminergic dysfunction.

8. The method of claim 5, wherein the hallucinations comprise a visual, auditory, tactile, gustatory or olfactory hallucination.

9. The method of claim 5, wherein the hallucinations are the result of:
(a) a neurodegenerative disorder;
(b) a psychiatric disorder;
(c) a neurological disorder;
(d) a brain tumor;
(e) a sleep disorder;
(f) a focal brain lesion;
(g) a diffuse involvement of the cerebral cortex;
(h) a sensory loss; and/or
(i) dysfunction of the enteric nervous system.

10. The method of claim 9, wherein:
(a) the neurodegenerative disorder is selected from the group consisting of synucleopathies, Parkinson's disease, Alzheimer's disease, dementia with Lewy bodies (DLB), multiple system atrophy (MSA), Huntington's Disease, Multiple Sclerosis (MS), Amyotorphic Lateral Sclerosis (ALS), schizophrenia, Friedreich's ataxia, vascular dementia, spinal muscular atrophy, supranuclear palsy, fronto temperal dementia (FTD), progressive supranuclear palsy, Guadeloupian Parkinsonism, parkinsonism, spinocerebellar ataxia, autism, stroke, traumatic brain injury, sleep disorders such as REM sleep behavior disorder (RBD), depression, down syndrome, Gaucher's disease (GD), Krabbe's disease (KD), lysosomal conditions affecting glycosphingolipid metabolism, ADHD, agitation, anxiety, delirium, irritability, illusion and delusions, amnesia, apathy, bipolar disorder, disinhibition, aberrant motor and obsessive-compulsive behaviors, addiction, cerebral palsy, epilepsy, major depressive disorder, degenerative processes associated with aging, and dementia of aging;
(b) the psychiatric disorder is selected from the group consisting of bipolar disorder, borderline personality disorder, depression (mixed), dissociative identity disorder, generalized anxiety disorder, major depression, obsessive compulsive disorder, post-traumatic stress disorder, psychosis (NOS), schizoaffective disorder, and schizophrenia;
(c) the focal brain lesion comprises occipital lobe lesions or temporal lobe lesions;
(d) the focal brain lesion comprises a temporal lobe lesion which is selected from the group consisting of lesions of the uncinate gyrus, cerebral peduncles, and substantia nigra;
(e) the diffuse involvement of the cerebral cortex is caused by a viral infectious disease;
(f) the diffuse involvement of the cerebral cortex is caused by a viral infectious disease and the viral infectious disease is selected from the group consisting of acute metabolic encephalopathies, encephalitis, and meningitis
(g) the diffuse involvement of the cerebral cortex is a result of a cerebral vasculitis condition; or
(h) the diffuse involvement of the cerebral cortex is a result of a cerebral vasculitis condition and the cerebral vasculitis condition is caused by an autoimmune disorder such as Systemic Lupus Erythematosus (SLE), a bacterial or viral infection, or a systemic vasculitis.

11. The method of claim 9, where the sensory loss is:
(a) visual;
(b) auditory;
(c) gustatory;
(d) tactile; and/or
(e) olfactory.

12. The method of claim 9, wherein the amino sterol reverses dysfunction:
(a) of the neurodegenerative disorder and treats and/or prevents the hallucinations and/or related symptom;
(b) of the psychiatric disorder and treats and/or prevents the hallucinations and/or related symptom;
(c) of the neurological disorder and treats and/or prevents the hallucination;
(d) of the sensory loss and treats the hallucination; and/or
(e) of the enteric nervous system and treats the hallucination.

13. The method of claim 1 or 5, wherein:
(a) the method results in a decreased number or severity of hallucinations of the subject; and/or
(b) the method results in the subject being hallucination-free; and/or
(c) the method results in a decrease in number of hallucinations, and the decrease in number of hallucinations comprises a reduction in number of hallucinations over a defined period of time;
(d) wherein the method results in a decreased severity of hallucinations over a defined period of time, wherein the decreased severity of hallucinations is measured by a medically recognized technique selected from the group consisting of Chicago Hallucination Assessment Tool (CHAT), The Psychotic Symptom Rating Scales (PSYRATS), Auditory Hallucinations Rating Scale (AHRS), Hamilton Program for Schizophrenia Voices Questionnaire (HPSVQ), Characteristics of Auditory Hallucinations Questionnaire (CAHQ), Mental Health Research Institute Unusual Perception Schedule (MUPS), positive and negative syndrome scale (PANSS), scale for the assessment of positive symptoms (SAPS), Launay-Slade hallucinations scale (LSHS), the Cardiff anomalous perceptions scale (CAPS), and structured interview for assessing perceptual anomalies (SIAPA);
(e) the defined period of time of (c) or (d) is independently about 1 day to about 10 days, about 10 days to about 30 days, about 30 days to about 3 months, about 3 months to about 6 months, about 6 months to about 12 months, or about greater than 12 months; and/or
(f) the defined period of time of (c) or (d) is independently selected from about 1 day, about 1 week, about 2 weeks, about 3 weeks, about 1 month, about 1.5 months, about 2 months, about 2.5 months, about 3 months, about 3.5 months, about 4 months, about 4.5 months, about 5 months, about 5.5 months, or about 6 months.

14. The method of claim 5, wherein the amino sterol or a salt or derivative thereof is administered orally, intranasally, or a combination thereof.

15. The method of claim 14, wherein the amino sterol or a salt or derivative thereof is administered orally and:
(a) the starting dose of the aminosterol or a salt or derivative thereof ranges from about 1 mg up to about 175 mg/day;
(b) the dose of the amino sterol or a salt or derivative thereof for the subject following escalation is fixed at a range of from about 1 mg up to about 500 mg/day; and/or
(c) the dose of the amino sterol or a salt or derivative thereof is escalated in about 25 mg increments.

16. The method of claim 14, wherein the amino sterol or a salt or derivative thereof is administered intranasally and:
(a) the starting dose of the aminosterol or a salt or derivative thereof ranges from about 0.001 mg to about 3 mg/day;
(b) the dose of the amino sterol or a salt or derivative thereof for the subject following escalation is fixed at a range of from about 0.001 mg up to about 6 mg/day;
(c) the dose of the amino sterol or a salt or derivative thereof for the subject following escalation is a dose which is subtherapeutic when given orally or by injection; and/or
(d) the dose of the amino sterol or a salt or derivative thereof is escalated in increments of about 0.1, about 0.2, about 0.25, about 0.3, about 0.35, about 0.4, about 0.45, about 0.5, about 0.55, about 0.6, about 0.65, about 0.7, about 0.75, about 0.8, about 0.85, about 0.9, about 0.95, about 1, about 1.1, about 1.2, about 1.3, about 1.4, about 1.5, about 1.6, about 1.7, about 1.8, about 1.9, or about 2 mg.

17. The method of claim 5, wherein:
(a) the dose of the amino sterol or a salt or derivative thereof is escalated every about 3 to about 5 days;
(b) the dose of the amino sterol or a salt or derivative thereof is escalated every about 1, about 2, about 3, about 4, about 5, about 6, about 7, about 8, about 9, about 10, about 11, about 12, about 13, or about 14 days;
(c) the dose of the amino sterol or a salt or derivative thereof is escalated about 1×/week, about 2×/week, about every other week, or about 1×/month;
(d) the fixed dose of the aminosterol or a salt or derivative thereof is administered once per day, every other day, once per week, twice per week, three times per week, four times per week, five times per week, six times per week, every other week, or every few days;
(e) the fixed dose of the aminosterol or a salt or derivative thereof is administered for a first time period of administration, followed by a cessation of administration for a second time period, followed by resuming administration upon recurrence of hallucinations or a symptom of hallucinations;
(f) the fixed dose of the aminosterol or a salt or derivative thereof is incrementally reduced after the fixed dose of aminosterol or a salt or derivative thereof has been administered to the subject for a period of time;
(g) the fixed dose of the aminosterol or a salt or derivative thereof is varied plus or minus a defined amount to enable a modest reduction or increase in the fixed dose; and/or
(h) the fixed dose of the aminosterol or a salt or derivative thereof is varied plus or minus a defined amount to enable a modest reduction or increase in the fixed dose and the fixed dose of the aminosterol or a salt or derivative thereof is increased or decreased by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20%.

18. The method of claim 5, wherein the starting dose of the aminosterol or a salt or derivative thereof is higher if the symptom being evaluated is severe.

19. The method of claim 5, wherein:
(a) progression or onset of the hallucinations and/or related symptoms is slowed, halted, or reversed over a defined period of time following administration of the fixed escalated dose of the aminosterol or a salt or derivative thereof, as measured by a medically-recognized technique;
(b) the hallucinations and/or related symptoms are positively impacted by administration of the aminosterol or a salt or derivative thereof, as measured by a medically-recognized technique;
(c) the positive impact and/or progression of hallucinations and/or related symptom is measured quantitatively or qualitatively by one or more medically recognized techniques selected from the group consisting of Chicago Hallucination Assessment Tool (CHAT), The Psychotic Symptom Rating Scales (PSYRATS), Auditory Hallucinations Rating Scale (AHRS), Hamilton Program for Schizophrenia Voices Questionnaire (HPSVQ), Characteristics of Auditory Hallucinations Questionnaire (CAHQ), Mental Health Research Institute Unusual Perception Schedule (MUPS), positive and negative syndrome scale (PANSS), scale for the assessment of positive symptoms (SAPS), Launay-Slade hallucinations scale (LSHS), the Cardiff anomalous perceptions scale (CAPS), and structured interview for assessing perceptual anomalies (SIAPA); and/or
(d) the progression or onset of hallucinations and/or related symptoms is slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by the one or more medically recognized techniques.

20. The method of claim 5, wherein the fixed escalated dose of the amino sterol or a salt or derivative thereof:
(a) reverses dysfunction caused by the hallucinations and treats, prevents, improves, and/or resolves the symptom being evaluated;
(b) reverses dysfunction caused by the hallucinations and treats, prevents, improves, and/or resolves the symptom being evaluated and the improvement or resolution of the hallucination symptom is measured using a clinically recognized scale or tool; and/or
(c) reverses dysfunction caused by the hallucinations and treats, prevents, improves, and/or resolves the symptom being evaluated and the hallucinations, by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, as measured using a clinically recognized scale.

21. The method of claim 5, wherein the hallucination symptom to be evaluated is selected from the group consisting of:
(a) a symptom from the Chicago Hallucination Assessment Tool (CHAT) selected from the group consisting of hallucination frequency, duration, sensory intensity, complexity, controllability, amount of negative content, degree of negative content, frequency of negative emotion associated with hallucination, intensity of emotional impact, and chronicity;
(b) a symptom from the Mental Health Research Institute Unusual Perceptions Schedule (MUPS) selected from the group consisting of onset and course, number, volume, tone, and location;
(c) auditory hallucination;
(d) tactile hallucination;
(e) visual hallucination;
(f) olfactory hallucination;
(g) gustatory hallucination;
(h) delusions;
(i) proprioceptive hallucination;
(j) equilibrioceptive hallucination;
(k) nociceptive hallucination;
(l) thermoceptive hallucination;
(m) chronoceptive hallucination;
(n) non-auditory command hallucination;
(o) psychosis;
(p) peduncular hallucinosis;
(p) delirium;
(r) dementia;
(s) neurodegenerative disease;
(t) neurodegeneration;
(u) epilepsy;
(v) seizures;
(w) migraines;
(x) cognitive impairment;
(y) constipation;
(z) depression;
(aa) sleep problem, sleep disorder, or sleep disturbance; and/or
(bb) gastrointestinal disorders.

22. The method of claim 21, wherein the hallucination symptom to be evaluated is visual hallucination and wherein:
(a) the method results in a decrease in number of visual hallucinations over a defined period of time;
(b) the method results in a decrease in severity of visual hallucinations over a defined period of time, wherein the decrease in severity of visual hallucinations is measured quantitatively or qualitatively by one or more medically recognized techniques selected from the group consisting of Chicago Hallucination Assessment Tool (CHAT), The Psychotic Symptom Rating Scales (PSYRATS), Hamilton Program for Schizophrenia Voices Questionnaire (HPSVQ), Mental Health Research Institute Unusual Perception Schedule (MUPS), positive and negative syndrome scale (PANSS), scale for the assessment of positive symptoms (SAPS), Launay-Slade hallucinations scale (LSHS), the Cardiff anomalous perceptions scale (CAPS), and structured interview for assessing perceptual anomalies (SIAPA);
(c) the method results in the subject being visual hallucination-free;
(d) the defined period of time is about 1 day to about 10 days, about 10 days to about 30 days, about 30 days to about 3 months, about 3 months to about 6 months, about 6 months to about 12 months, or about greater than 12 months;
(e) the decrease in number is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; and/or
(f) the decrease in severity is measured quantitatively and is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

23. The method of claim 21, wherein the hallucination symptom to be evaluated is auditory hallucination and wherein:
(a) the method results in a decrease in number of auditory hallucinations over a defined period of time;
(b) the method results in a decrease in severity of auditory hallucinations over a defined period of time, wherein the decrease in severity of auditory hallucinations is measured quantitatively or qualitatively by one or more medically recognized techniques selected from the group consisting of Chicago Hallucination Assessment Tool (CHAT), The Psychotic Symptom Rating Scales (PSYRATS), Auditory Hallucinations Rating Scale (AHRS), Hamilton Program for Schizophrenia Voices Questionnaire (HPSVQ), Characteristics of Auditory Hallucinations Questionnaire (CAHQ), Mental Health Research Institute Unusual Perception Schedule (MUPS), positive and negative syndrome scale (PANSS), scale for the assessment of positive symptoms (SAPS), Launay-Slade hallucinations scale (LSHS), the Cardiff anomalous perceptions scale (CAPS), and structured interview for assessing perceptual anomalies (SIAPA);
(c) the method results in the subject being auditory hallucination-free;
(d) the defined period of time is about 1 day to about 10 days, about 10 days to about 30 days, about 30 days to about 3 months, about 3 months to about 6 months, about 6 months to about 12 months, or about greater than 12 months;
(e) the decrease in number is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; and/or
(f) the decrease in severity is measured quantitatively and is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

24. The method of claim 21, wherein the hallucination symptom to be evaluated is tactile hallucination and wherein:
(a) the method results in a decrease in number of tactile hallucinations over a defined period of time;
(b) the method results in a decrease in severity of tactile hallucinations over a defined period of time, wherein the decrease in severity of tactile hallucinations is measured quantitatively or qualitatively by one or more medically recognized techniques selected from the group consisting of Chicago Hallucination Assessment Tool (CHAT), The Psychotic Symptom Rating Scales (PSYRATS), Hamilton Program for Schizophrenia Voices Questionnaire (HPSVQ), Mental Health Research Institute Unusual Perception Schedule (MUPS), positive and negative syndrome scale (PANSS), scale for the assessment of positive symptoms (SAPS), Launay-Slade hallucinations scale (LSHS), the Cardiff anomalous perceptions scale (CAPS), and structured interview for assessing perceptual anomalies (SIAPA);
(c) the method results in the subject being tactile hallucination-free;
(d) the defined period of time is about 1 day to about 10 days, about 10 days to about 30 days, about 30 days to about 3 months, about 3 months to about 6 months, about 6 months to about 12 months, or about greater than 12 months;
(e) the decrease in number is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; and/or
(f) the decrease in severity is measured quantitatively and is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

25. The method of claim 21, wherein the hallucination symptom to be evaluated is olfactory hallucination and wherein:
(a) the method results in a decrease in number of olfactory hallucinations over a defined period of time;
(b) the method results in a decrease in severity of olfactory hallucinations over a defined period of time, wherein the decrease in severity of olfactory hallucinations is measured quantitatively or qualitatively by one or more medically recognized techniques selected from the group consisting of Chicago Hallucination Assessment Tool (CHAT), The Psychotic Symptom Rating Scales (PSYRATS), Hamilton Program for Schizophrenia Voices Questionnaire (HPSVQ), Mental Health Research Institute Unusual Perception Schedule (MUPS), positive and negative syndrome scale (PANSS), scale for the assessment of positive symptoms (SAPS), Launay-Slade hallucinations scale (LSHS), the Cardiff anomalous perceptions scale (CAPS), and structured interview for assessing perceptual anomalies (SIAPA);
(c) the method results in the subject being olfactory hallucination-free;
(d) the defined period of time is about 1 day to about 10 days, about 10 days to about 30 days, about 30 days to about 3 months, about 3 months to about 6 months, about 6 months to about 12 months, or about greater than 12 months;
(e) the decrease in number is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; and/or
(f) the decrease in severity is measured quantitatively and is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

26. The method of claim 21, wherein the hallucination symptom to be evaluated is cognitive impairment, and wherein:
(a) progression or onset of the cognitive impairment is slowed, halted, or reversed over a defined period of time following administration of the fixed escalated dose of the aminosterol or a salt or derivative thereof, as measured by a medically-recognized technique; and/or
(b) the cognitive impairment is positively impacted by the fixed escalated dose of the aminosterol or a salt or derivative thereof, as measured by a medically-recognized technique;
(c) the cognitive impairment is positively impacted by the fixed escalated dose of the aminosterol or a salt or derivative thereof, as measured by a medically-recognized technique and the positive impact on and/or progression of cognitive impairment is measured quantitatively or qualitatively by one or more techniques selected from the group consisting of ADASCog, Mini-Mental State Exam (MMSE), Mini-cog test, Woodcock-Johnson Tests of Cognitive Abilities, Leiter International Performance Scale, Miller Analogies Test, Raven's Progressive Matrices, Wonderlic Personnel Test, IQ tests, or a computerized tested selected from Cantab Mobile, Cognigram, Cognivue, Cognision, and Automated Neuropsychological Assessment Metrics Cognitive Performance Test (CPT);
(d) the progression or onset of cognitive impairment is slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique; and/or
(e) each defined period of time is independently selected from the group consisting of about 1 day to about 10 days, about 10 days to about 30 days, about 30 days to about 3 months, about 3 months to about 6 months, about 6 months to about 12 months, and about greater than 12 months.

27. The method of claim 21, wherein the hallucination symptom to be evaluated is constipation, and wherein:
(a) the fixed escalated dose of the amino sterol or a salt or derivative thereof causes the subject to have a bowel movement;
(b) the method results in an increase in the frequency of bowel movement in the subject;
(c) the method results in an increase in the frequency of bowel movement in the subject and the increase in the frequency of bowel movement is defined as:
(i) an increase in the number of bowel movements per week of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or
(ii) a percent decrease in the amount of time between each successive bowel movement selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%;
(d) as a result of the method the subject has the frequency of bowel movement recommended by a medical authority for the age group of the subject; and/or
(e) the starting dose of the aminosterol or a salt or derivative thereof is determined by the severity of the constipation, wherein:
(i) if the average complete spontaneous bowel movement (CSBM) or spontaneous bowel movement (SBM) is one or less per week, then the starting amino sterol dose is at least about 150 mg; and
(ii) if the average CSBM or SBM is greater than one per week, then the starting amino sterol dose is about 75 mg or less.

28. The method of claim 21, wherein the hallucination symptom to be evaluated is a sleep problem, sleep disorder, and/or sleep disturbance, and wherein:
(a) treating the sleep problem, sleep disorder, sleep disturbance prevents or delays the onset and/or progression of the hallucination and/or related symptom;
(b) the sleep disorder or sleep disturbance comprises a delay in sleep onset, sleep fragmentation, REM-behavior disorder, sleep-disordered breathing including snoring and apnea, day-time sleepiness, micro-sleep episodes, narcolepsy, hallucinations, or any combination thereof, and optionally where the REM-behavior disorder comprises vivid dreams, nightmares, and acting out the dreams by speaking or screaming, or fidgeting or thrashing of arms or legs during sleep;
(d) the method results in a positive change in the sleeping pattern of the subject;
(e) the method results in a positive change in the sleeping pattern of the subject, wherein the positive change is defined as:
(i) an increase in the total amount of sleep obtained of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or
(ii) a percent decrease in the number of awakenings during the night selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; and/or
(f) as a result of the method the subject obtains the total number of hours of sleep recommended by a medical authority for the age group of the subject.

29. The method of claim 21, wherein the hallucination symptom to be evaluated is depression and wherein:
(a) treating the depression prevents and/or delays the onset and/or progression of the hallucinations and/or related symptom;
(b) the method results in improvement in a subject's depression, as measured by one or more clinically-recognized depression rating scale;
(c) the method results in improvement in a subject's depression, as measured by one or more clinically-recognized depression rating scale and the improvement is in one or more depression characteristics selected from the group consisting of mood, behavior, bodily functions such as eating, sleeping, energy, and sexual activity, and/or episodes of sadness or apathy; and/or
(d) the method results in improvement in a subject's depression, as measured by one or more clinically-recognized depression rating scales selected from the group consisting of Patient Health Questionnaire-9 (PHQ-9), the Beck Depression Inventory (BDI), Zung Self-Rating Depression Scale, Center for Epidemiologic Studies-Depression Scale (CES-D), and the Hamilton Rating Scale for Depression (HRSD); and the improvement a subject experiences following treatment is about 5, about 10, about 15, about 20, about 25, about 30, about 35, about 40, about 45, about 50, about 55, about 60, about 65, about 70, about 75, about 80, about 85, about 90, about 95 or about 100%.

30. The method of claim 21, wherein the hallucination symptom to be evaluated is neurodegeneration correlated with hallucinations, and wherein:
(a) treating the neurodegeneration prevents and/or delays the onset and/or progression of the hallucinations and/or related symptom;
(b) the method results in treating, preventing, and/or delaying the progression and/or onset of neurodegeneration in the subject;
(c) progression or onset of the neurodegeneration is slowed, halted, or reversed over a defined period of time following administration of the fixed escalated dose of the amino sterol or a salt or derivative thereof, as measured by a medically-recognized technique; and/or
(d) the neurodegeneration is positively impacted by the fixed escalated dose of the aminosterol or a salt or derivative thereof, as measured by a medically-recognized technique;
(e) the positive impact and/or progression of neurodegeneration is measured quantitatively or qualitatively by one or more techniques selected from the group consisting of electroencephalogram (EEG), neuroimaging, functional MRI, structural MRI, diffusion tensor imaging (DTI), [18F]fluorodeoxyglucose (FDG) PET, agents that label amyloid, [18F]F-dopa PET, radiotracer imaging, volumetric analysis of regional tissue loss, specific imaging markers of abnormal protein deposition, multimodal imaging, and biomarker analysis;
(f) the progression or onset of neurodegeneration is slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique; and/or
(g) each defined period of time is independently selected from the group consisting of about 1 day to about 10 days, about 10 days to about 30 days, about 30 days to about 3 months, about 3 months to about 6 months, about 6 months to about 12 months, and about greater than 12 months.

31. The method of claim 1 or 5, wherein the aminosterol or a salt or derivative thereof is administered in combination with at least one additional active agent to achieve either an additive or synergistic effect.

32. The method of claim 31, wherein:
(a) the additional active agent is administered via a method selected from the group consisting of concomitantly, as an admixture, separately and simultaneously or concurrently, and separately and sequentially;
(b) the additional active agent is a second amino sterol;
(c) the method comprises a first aminosterol which is aminosterol 1436 or a salt or derivative thereof administered intranasally and a second aminosterol which is squalamine or a salt or derivative thereof administered orally;
(d) the additional active agent is an active agent used to treat hallucination or a symptom thereof; and/or
(e) the additional active agent is an active agent used to treat hallucination or a symptom thereof which is selected from the group consisting of first-generation antipsychotics such as chlorpromazine (Thorazine®), fluphenazine (Prolixin®), haloperidol (Haldol®), perphenazine (Trilafon®), thioridazine (Mellaril®), thiothixene (Navane®), and trifluoperazine (Stelazine®); atypical antipsychotics such as aripiprazole (Abilify®), aripiprazole lauroxil (Aristada®), asenapine (Saphris®), clozapine (Clozaril®), iloperidone (Fanapt®), lurasidone (Latuda®), olanzapine (Zyprexa®), paliperidone (Invega Sustenna®), paliperidone palmitate (Invega Trinza®), quetiapine (Seroquel®), risperidone (Risperdal®), pimavanserin and ziprasidone (Geodon®).

33. The method of claim 1 or 5, wherein:
(a) each aminosterol dose is administered on an empty stomach, optionally within two hours of the subject waking; and/or
(b) no food is taken after about 60 to about 90 minutes of taking the aminosterol dose.

34. The method of claim 1 or 5, wherein the aminosterol or the salt or derivative thereof is:
(a) isolated from the liver of Squalus acanthias;
(b) squalamine or a pharmaceutically acceptable salt thereof;
(c) a squalamine isomer or a pharmaceutically acceptable salt thereof;
(d) a phosphate salt of squalamine or a pharmaceutically acceptable salt thereof;
(e) aminosterol 1436 or a pharmaceutically acceptable salt thereof;
(f) an isomer of amino sterol 1436 or a pharmaceutically acceptable salt thereof;
(g) a phosphate salt of aminosterol 1436;
(h) comprises a sterol nucleus and a polyamine attached at any position on the sterol, such that the molecule exhibits a net charge of at least +1;
(i) comprises a bile acid nucleus and a polyamine, attached at any position on the bile acid, such that the molecule exhibits a net charge of at least +1;
(j) a derivative modified to include one or more of the following:
(i) substitutions of the sulfate by a sulfonate, phosphate, carboxylate, or other anionic moiety chosen to circumvent metabolic removal of the sulfate moiety and oxidation of the cholesterol side chain;
(ii) replacement of a hydroxyl group by a non-metabolizable polar substituent, such as a fluorine atom, to prevent its metabolic oxidation or conjugation; and
(iii) substitution of one or more ring hydrogen atoms to prevent oxidative or reductive metabolism of the steroid ring system;
(k) a derivative of squalamine modified through medicinal chemistry to improve bio-distribution, ease of administration, metabolic stability, or any combination thereof; and/or
(l) a synthetic amino sterol.

35. The method of claim 1 or 5, wherein the aminosterol is selected from the group consisting of:

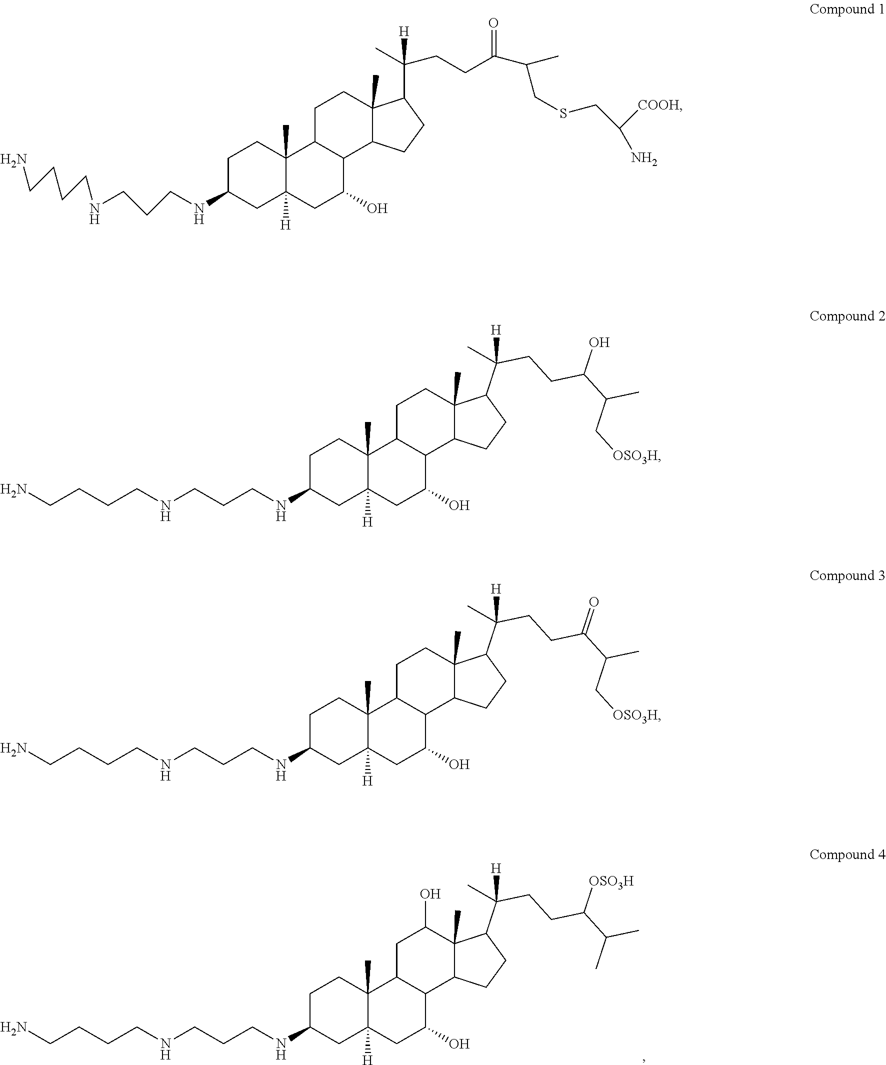

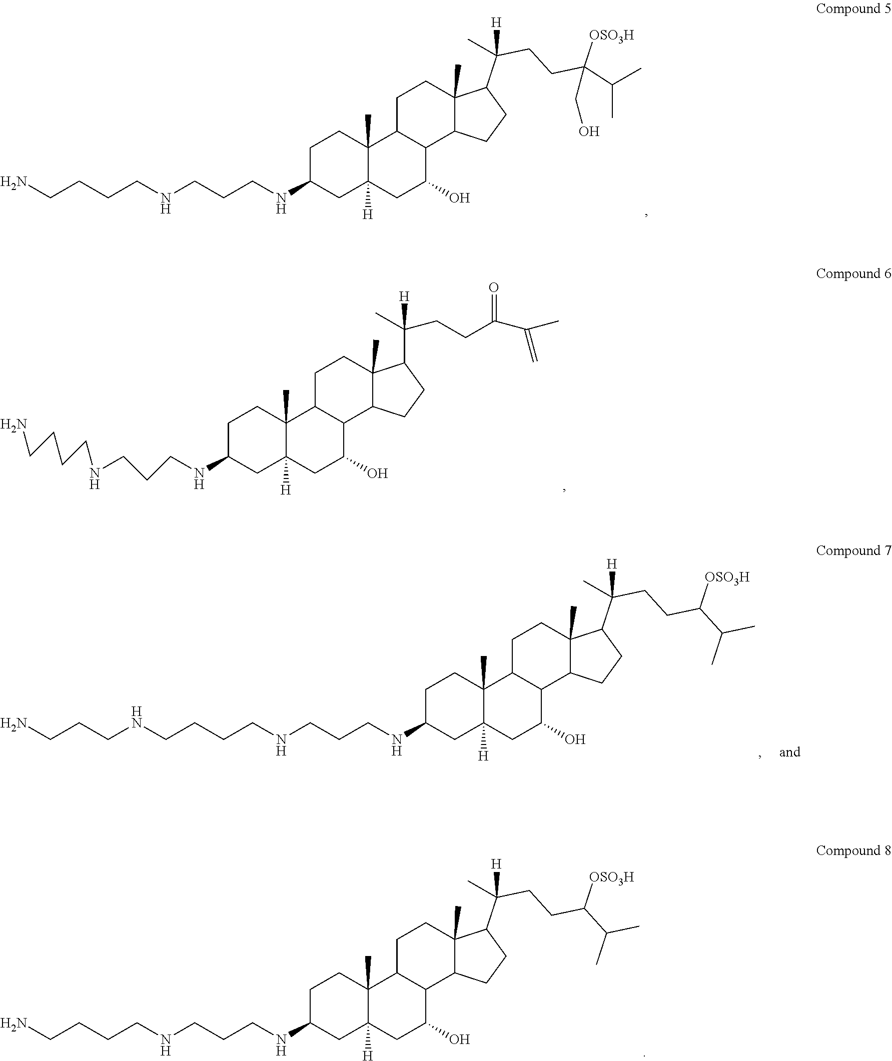

36. The method of claim 1 or 5, wherein the aminosterol is selected from the group consisting aminosterol 1436 or a pharmaceutically acceptable salt thereof, squalamine or a pharmaceutically acceptable salt thereof, or a combination thereof.

37. The method of claim 1 or 5, wherein the aminosterol is a phosphate salt.

38. The method of claim 1 or 5, wherein the aminosterol is comprised in a composition comprising one or more of the following:
(a) an aqueous carrier;
(b) a buffer;
(c) a sugar; and/or
(d) a polyol compound.

39. The method of claim 1 or 5, wherein the subject is a human.

40. The method of claim 1 or 5, wherein the subject is a member of a patient population or individual at risk for hallucinations.
